SEQUENCE LISTING (43 sequences)

SEQ ID NO: 1 - DNA, 1617 bp, Synthetic construct
  source 1..1617: mol_type = other DNA
atggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtc

SEQ ID NO: 2 - DNA, 510 bp, Synthetic construct
  source 1..510: mol_type = other DNA
atggctcacaatgacaacaccccgcactcccgccgtaccggcgatgcggccgtgaccggtattacgcgtcgccagtggctgcaaggcgcgctggccctgaccgcagctggcctgacgggttccctggccctgcgcgcactggctgatgatccgggcaccgcaccgctggatacctttatgacgctgagcgaagctctgacgggcaaaaaaggtctgtctcgtgttctgggccagcgttttctgcaagcgctgcaaaaaggttcattcaaaaccgcggattcgctgccgcagctggcgggcgccctggcaagcggttctctgaacccggaccaagaagctctggcgctgaaaatcctggaagcatggtatctgggcattgttgataatgtggttatcacctacgaagaagccctgatgtttagtgtcgtgtccgacacgctggtcattccgagctattgcccgaacaaaccgggtttctgggccgaaaaaccgatcgaacgtcaggcataa

SEQ ID NO: 3 - DNA, 69 bp, Synthetic construct
  source 1..69: mol_type = other DNA
attcgtgcaggtgctaccatgccgcatcgtgatcgtggtccgtgcggtgcatgtcacgctattatccag

SEQ ID NO: 4 - DNA, 39 bp, Synthetic construct
  source 1..39: mol_type = other DNA
gaattcggttctggttatggctctggtccgccgggtccg

SEQ ID NO: 5 - DNA, 2191 bp, Synthetic construct
  source 1..2191: mol_type = other DNA
ctcacaatgacaacaccccgcactcccgccgtaccggcgatgcggccgtgaccggtattacgcgtcgccagtggctgcaaggcgcgctggccctgaccgcagctggcctgacgggttccctggccctgcgcgcactggctgatgatccgggcaccgcaccgctggatacctttatgacgctgagcgaagctctgacgggcaaaaaaggtctgtctcgtgttctgggccagcgttttctgcaagcgctgcaaaaaggttcattcaaaaccgcggattcgctgccgcagctggcgggcgccctggcaagcggttctctgaacccggaccaagaagctctggcgctgaaaatcctggaagcatggtatctgggcattgttgataatgtggttatcacctacgaagaagccctgatgtttagtgtcgtgtccgacacgctggtcattccgagctattgcccgaacaaaccgggtttctgggccgaaaaaccgatcgaacgtcaggcataatggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcattcgtgcaggtgctaccatgccgcatcgtgatcgtggtccgtgcggtgcatgtcacgctattatccag

SEQ ID NO: 6 - DNA, 2230 bp, Synthetic construct
  source 1..2230: mol_type = other DNA
ctcacaatgacaacaccccgcactcccgccgtaccggcgatgcggccgtgaccggtattacgcgtcgccagtggctgcaaggcgcgctggccctgaccgcagctggcctgacgggttccctggccctgcgcgcactggctgatgatccgggcaccgcaccgctggatacctttatgacgctgagcgaagctctgacgggcaaaaaaggtctgtctcgtgttctgggccagcgttttctgcaagcgctgcaaaaaggttcattcaaaaccgcggattcgctgccgcagctggcgggcgccctggcaagcggttctctgaacccggaccaagaagctctggcgctgaaaatcctggaagcatggtatctgggcattgttgataatgtggttatcacctacgaagaagccctgatgtttagtgtcgtgtccgacacgctggtcattccgagctattgcccgaacaaaccgggtttctgggccgaaaaaccgatcgaacgtcaggcataatggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgctaccatgccgcatcgtgatcgtggtccgtgcggtgcatgtcacgctattatccag

SEQ ID NO: 7 - DNA, 2272 bp, Synthetic construct
  source 1..2272: mol_type = other DNA
ctcacaatgacaacaccccgcactcccgccgtaccggcgatgcggccgtgaccggtattacgcgtcgccagtggctgcaaggcgcgctggccctgaccgcagctggcctgacgggttccctggccctgcgcgcactggctgatgatccgggcaccgcaccgctggatacctttatgacgctgagcgaagctctgacgggcaaaaaaggtctgtctcgtgttctgggccagcgttttctgcaagcgctgcaaaaaggttcattcaaaaccgcggattcgctgccgcagctggcgggcgccctggcaagcggttctctgaacccggaccaagaagctctggcgctgaaaatcctggaagcatggtatctgggcattgttgataatgtggttatcacctacgaagaagccctgatgtttagtgtcgtgtccgacacgctggtcattccgagctattgcccgaacaaaccgggtttctgggccgaaaaaccgatcgaacgtcaggcataatggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgctaccatgccgcatcgtgatcgtggtccgtgcggtgcatgtcacgctattatccagggcagtggttccggccatcaccatcaccatcactaaaagctt

SEQ ID NO: 8 - protein, 527 aa, Synthetic construct
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAAL

SEQ ID NO: 9 - protein, 562 aa, Synthetic construct
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVIRAGATMPHRDRGPCGACHAIIQ

SEQ ID NO: 10 - protein, 586 aa, Synthetic construct
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGATMPHRDRGPCGACHAIIQGSGSGHHHHHH

SEQ ID NO: 11 - protein, 575 aa, Synthetic construct
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGATMPHRDRGPCGACHAIIQ

SEQ ID NO: 12 - protein, 586 aa, Synthetic construct
MADTDTQKADVVVVGSGVAGAIVAHQLAMAGKSVILLEAGPXMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDXAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRXRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGAXMXHRDRGPCGACHAIIQGSGSGHHHHHH

SEQ ID NO: 13 - protein, 575 aa, Synthetic construct
  MOD_RES 42: note = R or a non-canonical amino acid
  MOD_RES 247: note = S or a non-canonical amino acid
  MOD_RES 395: note = D or a non-canonical amino acid
  MOD_RES 558: note = T or a non-canonical amino acid
  MOD_RES 560: note = P or a non-canonical amino acid
MADTDTQKADVVVVGSGVAGAIVAHQLAMAGKSVILLEAGPXMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDXAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRXRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGAXMXHRDRGPCGACHAIIQ

SEQ ID NO: 14 - DNA, 1727 bp, Synthetic construct
  source 1..1727: mol_type = other DNA
tggcggatacggatacccagaaagcggacgtggtcgtggttggatccggcgtggcaggcgcaatcgtggctcatcaactggcaatggcaggtaaaagcgtgatcctgctggaagctggtccgtagatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgctaccatgccgcatcgtgatcgtggtccgtgcggtgcatgtcacgctattatccagtaa

SEQ ID NO: 15 - DNA, 1728 bp, Synthetic construct
  source 1..1728: mol_type = other DNA
atggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgattaggcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgctaccatgccgcatcgtgatcgtggtccgtgcggtgcatgtcacgctattatccagtaa

SEQ ID NO: 16 - DNA, 1729 bp, Synthetic construct
  source 1..1729: mol_type = other DNA
atggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgttagcgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgctaccatgccgcatcgtgatcgtggtccgtgcggtgcatgtcacgctattatccaggtaa

SEQ ID NO: 17 - DNA, 1728 bp, Synthetic construct
  source 1..1728: mol_type = other DNA
atggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgcttagatgccgcatcgtgatcgtggtccgtgcggtgcatgtcacgctattatccagtaa

SEQ ID NO: 18 - DNA, 1729 bp, Synthetic construct
  source 1..1729: mol_type = other DNA
atggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgctaccatgtagcatcgtgatcgtggtccgtgcggtgcatgtcacgctattatccagtaaa

SEQ ID NO: 19 - protein, 575 aa, Synthetic construct
  MOD_RES 42: note = Non-canonical amino acid
MADTDTQKADVVVVGSGVAGAIVAHQLAMAGKSVILLEAGPXMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGATMPHRDRGPCGACHAIIQ

SEQ ID NO: 20 - protein, 575 aa, Synthetic construct
  MOD_RES 247: note = Non-canonical amino acid
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDXAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGATMPHRDRGPCGACHAIIQ

SEQ ID NO: 21 - protein, 575 aa, Synthetic construct
  MOD_RES 395: note = Non-canonical amino acid
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRXRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGATMPHRDRGPCGACHAIIQ

SEQ ID NO: 22 - protein, 575 aa, Synthetic construct
  MOD_RES 558: note = Non-canonical amino acid
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGAXMPHRDRGPCGACHAIIQ

SEQ ID NO: 23 - protein, 575 aa, Synthetic construct
  MOD_RES 560: note = Non-canonical
MADTDTQKADVVVVGSGVAGAIVAHQLAMAGKSVILLEAGPRMPRWEIVERFRNQVDKTDFMAPYPSSAWAPHPEYGPPNDYLILKGEHKFNSQYIRAVGGTTWHWAASAWRFIPNDFKMKTVYGVGRDWPIQYDDIEHYYQRAEEELGVWGPGPEEDLYSPRKEPYPMPPLPLSFNEQTIKSALNGYDPKFHVVTEPVARNSRPYDGRPTCCGNNNCMPICPIGAMYNGIVHVEKAEQAGAKLIDSAVVYKLETGPDKRITAAVYKDKTGADHRVEGKYFVIAANGIETPKILLMSANRDFPNGVANSSDMVGRNLMDHPGTGVSFYANEKLWPGRGPQEMTSLIGFRDGPFRANEAAKKIHLSNMSRINQETQKIFKGGKLMKPEELDAQIRDRSARFVQFDCFHEILPQPENRIVPSKTATDAVGIPRPEITYAIDDYVKRGAVHTREVYATAAKVLGGTEVVFNDEFAPNNHITGATIMGADARDSVVDKDCRAFDHPNLFISSSSTMPTVGTVNVTLTIAALALRMSDTLKKEVEFGSGYGSGPPGPIRAGATMXHRDRGPCGACHAIIQ

SEQ ID NO: 24 - DNA, 18 bp, Synthetic construct
  source 1..18: mol_type = other DNA
catcaccatcaccatcac

SEQ ID NO: 25 - DNA, 15 bp, Synthetic construct
  source 1..15: mol_type = other DNA
ggcagtggttccggc

SEQ ID NO: 26 - DNA, 2278 bp, Synthetic construct
  source 1..2278: mol_type = other DNA
ccatggctcacaatgacaacaccccgcactcccgccgtaccggcgatgcggccgtgaccggtattacgcgtcgccagtggctgcaaggcgcgctggccctgaccgcagctggcctgacgggttccctggccctgcgcgcactggctgatgatccgggcaccgcaccgctggatacctttatgacgctgagcgaagctctgacgggcaaaaaaggtctgtctcgtgttctgggccagcgttttctgcaagcgctgcaaaaaggttcattcaaaaccgcggattcgctgccgcagctggcgggcgccctggcaagcggttctctgaacccggaccaagaagctctggcgctgaaaatcctggaagcatggtatctgggcattgttgataatgtggttatcacctacgaagaagccctgatgtttagtgtcgtgtccgacacgctggtcattccgagctattgcccgaacaaaccgggtttctgggccgaaaaaccgatcgaacgtcaggcataatggcggatacggatacccagaaagcggacgtggtcgtggttggatccggcgtggcaggcgcaatcgtggctcatcaactggcaatggcaggtaaaagcgtgatcctgctggaagctggtccgcgtatgccgcgttgggaaattgttgaacgtttccgcaatcaagtcgataaaaccgactttatggcaccgtatccgagcagcgcatgggcaccgcatccggaatatggtccgccgaatgattacctgatcctgaaaggcgaacacaaatttaactcacagtacattcgtgcagtgggcggcaccacgtggcattgggcagcctcggcatggcgcttcatcccgaacgattttaaaatgaaaaccgtgtatggcgttggtcgtgactggccgattcagtacgatgacatcgaacattattaccaacgcgcggaagaagaactgggcgtgtggggtccgggcccggaagaagacctgtattcaccgcgtaaagaaccgtacccgatgccgccgctgccgctgagtttcaatgaacaaaccattaaatccgctctgaacggctatgatccgaaatttcacgtggttacggaaccggtggcccgtaattcgcgcccgtacgacggtcgcccgacctgctgtggcaacaataactgcatgccgatttgtccgatcggtgcaatgtataacggcatcgtccatgtggaaaaagctgaacaggcaggtgctaaactgattgatagtgcggtcgtgtacaaactggaaacgggcccggacaaacgtattaccgcagctgtttataaagataaaacgggtgcggaccatcgcgtcgaaggcaaatacttcgtgattgcggccaatggtatcgaaaccccgaaaattctgctgatgagcgcgaaccgtgattttccgaatggtgtggccaacagttccgatatggttggccgcaatctgatggaccatccgggcaccggcgtgagcttttatgcaaacgaaaaactgtggccgggtcgtggtccgcaggaaatgacctctctgatcggtttccgtgatggcccgtttcgcgcgaatgaagcagcgaagaaaattcatctgtcaaatatgtcgcgtatcaaccaggaaacccaaaaaatctttaaaggcggtaaactgatgaaaccggaagaactggatgcgcagatccgtgaccgcagtgcccgctttgttcaattcgattgctttcacgaaatcctgccgcagccggaaaatcgtattgtcccgtccaaaaccgcaacggacgcagtgggtattccgcgtccggaaattacgtatgcgatcgatgactacgtcaaacgtggcgcagtgcatacgcgcgaagtttatgctaccgcggccaaagtgctgggcggcaccgaagtggtcttcaacgatgaatttgcgccgaataaccacatcaccggtgccacgattatgggcgcggatgcccgtgactcagtggttgataaagactgtcgcgccttcgatcatccgaacctgtttattagcagcagcagcaccatgccgacggttggcaccgttaacgtcaccctgacgattgcagctctggcactgcgtatgtctgatacgctgaaaaaagaagtcgaattcggttctggttatggctctggtccgccgggtccgattcgtgcaggtgctaccatgccgcatcgtgatcgtggtccgtgcggtgcatgtcacgctattatccagggcagtggttccggccatcaccatcaccatcactaaaagctt

SEQ ID NO: 27 - DNA, 79 bp, Synthetic construct
  source 1..79: mol_type = other DNA
gctgccgaccgctgctgctggtctgctgctcctcgctgcccagccggcgatggccatgattcgtgcaggtgctaccatg

SEQ ID NO: 28 - DNA, 63 bp, Synthetic construct
  source 1..63: mol_type = other DNA
ggagcagcagaccagcagcagcggtcggcagcaggtatttcatggtatatctccttcttaaag

SEQ ID NO: 29 - DNA, 59 bp, Synthetic construct
  source 1..59: mol_type = other DNA
cggacccggcggaccagagccataaccagaaccgaattcctggataatagcgtgacatg

SEQ ID NO: 30 - DNA, 64 bp, Synthetic construct
  source 1..64: mol_type = other DNA
gaattcggttctggttatggctctggtccgccgggtccggagccgaaactggatatgaataaac

SEQ ID NO: 31 - DNA, 23 bp, Synthetic construct
  source 1..23: mol_type = other DNA
tgcatcctcaaattctgttaaag

SEQ ID NO: 32 - DNA, 51 bp, Synthetic construct
  source 1..51: mol_type = other DNA
ggacctactttaacagaatttgaggatgcaggcagtggttccggccatcac

SEQ ID NO: 33 - DNA, 21 bp, Synthetic construct
  source 1..21: mol_type = other DNA
ggtgaaactaaggaagatatc

SEQ ID NO: 34 - DNA, 51 bp, Synthetic construct
  source 1..51: mol_type = other DNA
cttttctagcgatatcttccttagtttcacccggacccggcggaccagagc

SEQ ID NO: 35 - DNA, 61 bp, Synthetic construct
  source 1..61: mol_type = other DNA
ctgctgctcctcgctgcccagccggcgatggccatggagccgaaactggatatgaataaac

SEQ ID NO: 36 - DNA, 65 bp, Synthetic construct
  source 1..65: mol_type = other DNA
gcgggcgaaatcttttgtttattcatatccagtttcggctccatggccatcgccggctgggcagc

SEQ ID NO: 37 - DNA, 65 bp, Synthetic construct
  source 1..65: mol_type = other DNA
gctgctcctcgctgcccagccggcgatggccatgggtgaaactaaggaagatatcgctagaaaag

SEQ ID NO: 38 - DNA, 55 bp, Synthetic construct
  source 1..55: mol_type = other DNA
gttcttttctagcgatatcttccttagtttcacccatggccatcgccggctgggc

SEQ ID NO: 39 - DNA, 1341 bp, Synthetic construct
  source 1..1341: mol_type = other DNA
atgaaatacctgctgccgaccgctgctgctggtctgctgctcctcgctgcccagccggcgatggccatgggtgaaactaaggaagatatcgctagaaaagaacaactaaaatcgctgctacctcctctagataatattattaacctttacgactttgaatacttggcctctcaaactttgactaaacaagcgtgggcctactattcctccggtgctaacgacgaagttactcacagagaaaaccataatgcttatcataggatttttttcaaaccaaagatccttgtagatgtacgcaaagtagacatttcaactgacatgttgggttctcatgtggatgttcccttctacgtgtctgctacagctttgtgtaaactgggaaaccccttagaaggtgaaaaagatgtcgccagaggttgtggccaaggtgtgacaaaagtcccacaaatgatatctactttggcttcatgttcccctgaggaaattattgaagcagcaccctctgataaacaaattcaatggtaccaactatatgttaactctgatagaaagatcactgatgatttggttaaaaatgtagaaaagctgggtgtaaaggcattatttgtcactgtggatgctccaagtttaggtcaaagagaaaaagatatgaagctgaaattttccaatacaaaggctggtccaaaagcgatgaagaaaactaatgtagaagaatctcaaggtgcttcgagagcgttatcaaagtttattgacccctctttgacttggaaagatatagaagagttgaagaaaaagacaaaactacctattgttatcaaaggtgttcaacgtaccgaagatgttatcaaagcagcagaaatcggtgtaagtggggtggttctatccaatcatggtggtagacaattagatttttcaagggctcccattgaagtcctggctgaaaccatgccaatcctggaacaacgtaacttgaaggataagttggaagttttcgtggacggtggtgttcgtcgtggtacagatgtcttgaaagcgttatgtctaggtgctaaaggtgttggtttgggtagaccattcttgtatgcgaactcatgctatggtcgtaatggtgttgaaaaagccattgaaattttaagagatgaaattgaaatgtctatgagactattaggtgttactagcattgcggaattgaagcctgatcttttagatctatcaacactaaaggcaagaacagttggagtaccaaacgacgtgctgtataatgaagtttatgagggacctactttaacagaatttgaggatgcaggcagtggttccggccatcaccatcaccatcactaa

SEQ ID NO: 40 - DNA, 1638 bp, Synthetic construct
  source 1..1638: mol_type = other DNA
atgaaatacctgctgccgaccgctgctgctggtctgctgctcctcgctgcccagccggcgatggccatggagccgaaactggatatgaataaacaaaagatttcgcccgctgaagttgccaagcataacaagcccgatgattgttgggttgtgatcaatggttacgtatacgacttaacgcgattcctaccaaatcatccaggtgggcaggatgttatcaagtttaacgccgggaaagatgtcactgctatttttgaaccactacatgctcctaatgtcatcgataagtatatagctcccgagaaaaaattgggtccccttcaaggatccatgcctcctgaacttgtctgtcctccttatgctcctggtgaaactaaggaagatatcgctagaaaagaacaactaaaatcgctgctacctcctctagataatattattaacctttacgactttgaatacttggcctctcaaactttgactaaacaagcgtgggcctactattcctccggtgctaacgacgaagttactcacagagaaaaccataatgcttatcataggatttttttcaaaccaaagatccttgtagatgtacgcaaagtagacatttcaactgacatgttgggttctcatgtggatgttcccttctacgtgtctgctacagctttgtgtaaactgggaaaccccttagaaggtgaaaaagatgtcgccagaggttgtggccaaggtgtgacaaaagtcccacaaatgatatctactttggcttcatgttcccctgaggaaattattgaagcagcaccctctgataaacaaattcaatggtaccaactatatgttaactctgatagaaagatcactgatgatttggttaaaaatgtagaaaagctgggtgtaaaggcattatttgtcactgtggatgctccaagtttaggtcaaagagaaaaagatatgaagctgaaattttccaatacaaaggctggtccaaaagcgatgaagaaaactaatgtagaagaatctcaaggtgcttcgagagcgttatcaaagtttattgacccctctttgacttggaaagatatagaagagttgaagaaaaagacaaaactacctattgttatcaaaggtgttcaacgtaccgaagatgttatcaaagcagcagaaatcggtgtaagtggggtggttctatccaatcatggtggtagacaattagatttttcaagggctcccattgaagtcctggctgaaaccatgccaatcctggaacaacgtaacttgaaggataagttggaagttttcgtggacggtggtgttcgtcgtggtacagatgtcttgaaagcgttatgtctaggtgctaaaggtgttggtttgggtagaccattcttgtatgcgaactcatgctatggtcgtaatggtgttgaaaaagccattgaaattttaagagatgaaattgaaatgtctatgagactattaggtgttactagcattgcggaattgaagcctgatcttttagatctatcaacactaaaggcaagaacagttggagtaccaaacgacgtgctgtataatgaagtttatgagggacctactttaacagaatttgaggatgcaggcagtggttccggccatcaccatcaccatcactaa

SEQ ID NO: 41 - DNA, 1449 bp, Synthetic construct
  source 1..1449: mol_type = other DNA
atgaaatacctgctgccgaccgctgctgctggtctgctgctcctcgctgcccagccggcgatggccatgattcgtgcaggtgctaccatgccgcatcgtgatcgtggtccgtgcggtgcatgtcacgctattatccaggaattcggttctggttatggctctggtccgccgggtccgggtgaaactaaggaagatatcgctagaaaagaacaactaaaatcgctgctacctcctctagataatattattaacctttacgactttgaatacttggcctctcaaactttgactaaacaagcgtgggcctactattcctccggtgctaacgacgaagttactcacagagaaaaccataatgcttatcataggatttttttcaaaccaaagatccttgtagatgtacgcaaagtagacatttcaactgacatgttgggttctcatgtggatgttcccttctacgtgtctgctacagctttgtgtaaactgggaaaccccttagaaggtgaaaaagatgtcgccagaggttgtggccaaggtgtgacaaaagtcccacaaatgatatctactttggcttcatgttcccctgaggaaattattgaagcagcaccctctgataaacaaattcaatggtaccaactatatgttaactctgatagaaagatcactgatgatttggttaaaaatgtagaaaagctgggtgtaaaggcattatttgtcactgtggatgctccaagtttaggtcaaagagaaaaagatatgaagctgaaattttccaatacaaaggctggtccaaaagcgatgaagaaaactaatgtagaagaatctcaaggtgcttcgagagcgttatcaaagtttattgacccctctttgacttggaaagatatagaagagttgaagaaaaagacaaaactacctattgttatcaaaggtgttcaacgtaccgaagatgttatcaaagcagcagaaatcggtgtaagtggggtggttctatccaatcatggtggtagacaattagatttttcaagggctcccattgaagtcctggctgaaaccatgccaatcctggaacaacgtaacttgaaggataagttggaagttttcgtggacggtggtgttcgtcgtggtacagatgtcttgaaagcgttatgtctaggtgctaaaggtgttggtttgggtagaccattcttgtatgcgaactcatgctatggtcgtaatggtgttgaaaaagccattgaaattttaagagatgaaattgaaatgtctatgagactattaggtgttactagcattgcggaattgaagcctgatcttttagatctatcaacactaaaggcaagaacagttggagtaccaaacgacgtgctgtataatgaagtttatgagggacctactttaacagaatttgaggatgcaggcagtggttccggccatcaccatcaccatcactaa

SEQ ID NO: 42 - DNA, 1746 bp, Synthetic construct
  source 1..1746: mol_type = other DNA
atgaaatacctgctgccgaccgctgctgctggtctgctgctcctcgctgcccagccggcgatggccatgattcgtgcaggtgctaccatgccgcatcgtgatcgtggtccgtgcggtgcatgtcacgctattatccaggaattcggttctggttatggctctggtccgccgggtccggagccgaaactggatatgaataaacaaaagatttcgcccgctgaagttgccaagcataacaagcccgatgattgttgggttgtgatcaatggttacgtatacgacttaacgcgattcctaccaaatcatccaggtgggcaggatgttatcaagtttaacgccgggaaagatgtcactgctatttttgaaccactacatgctcctaatgtcatcgataagtatatagctcccgagaaaaaattgggtccccttcaaggatccatgcctcctgaacttgtctgtcctccttatgctcctggtgaaactaaggaagatatcgctagaaaagaacaactaaaatcgctgctacctcctctagataatattattaacctttacgactttgaatacttggcctctcaaactttgactaaacaagcgtgggcctactattcctccggtgctaacgacgaagttactcacagagaaaaccataatgcttatcataggatttttttcaaaccaaagatccttgtagatgtacgcaaagtagacatttcaactgacatgttgggttctcatgtggatgttcccttctacgtgtctgctacagctttgtgtaaactgggaaaccccttagaaggtgaaaaagatgtcgccagaggttgtggccaaggtgtgacaaaagtcccacaaatgatatctactttggcttcatgttcccctgaggaaattattgaagcagcaccctctgataaacaaattcaatggtaccaactatatgttaactctgatagaaagatcactgatgatttggttaaaaatgtagaaaagctgggtgtaaaggcattatttgtcactgtggatgctccaagtttaggtcaaagagaaaaagatatgaagctgaaattttccaatacaaaggctggtccaaaagcgatgaagaaaactaatgtagaagaatctcaaggtgcttcgagagcgttatcaaagtttattgacccctctttgacttggaaagatatagaagagttgaagaaaaagacaaaactacctattgttatcaaaggtgttcaacgtaccgaagatgttatcaaagcagcagaaatcggtgtaagtggggtggttctatccaatcatggtggtagacaattagatttttcaagggctcccattgaagtcctggctgaaaccatgccaatcctggaacaacgtaacttgaaggataagttggaagttttcgtggacggtggtgttcgtcgtggtacagatgtcttgaaagcgttatgtctaggtgctaaaggtgttggtttgggtagaccattcttgtatgcgaactcatgctatggtcgtaatggtgttgaaaaagccattgaaattttaagagatgaaattgaaatgtctatgagactattaggtgttactagcattgcggaattgaagcctgatcttttagatctatcaacactaaaggcaagaacagttggagtaccaaacgacgtgctgtataatgaagtttatgagggacctactttaacagaatttgaggatgcaggcagtggttccggccatcaccatcaccatcactaa

SEQ ID NO: 43 - protein, 570 aa, Synthetic construct
MKYLLPTAAAGLLLLAAQPAMAMIRAGATMPHRDRGPCGACHAIIQEFGSGYGSGPPGPEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

(19) United States
(12) Patent Application Publication
(10) Pub. No.: US 2024/0141303 A1
(43) Pub. Date: May 2, 2024

(54) RECOMBINANT LACTATE DEHYDROGENASE AND USES THEREOF

(71) Applicant: B. G. NEGEV TECHNOLOGIES AND APPLICATIONS LTD., AT BEN-GURION UNIVERSITY, Beer Sheva (IL)

(72) Inventors: Lital ALFONTA, Beer Sheva (IL); Anastasya BOYARSKI, Beer Sheva (IL)

(73) Assignee: B. G. NEGEV TECHNOLOGIES AND APPLICATIONS LTD., AT BEN-GURION UNIVERSITY, Beer Sheva (IL)

(21) Appl. No.: 18/404,446

(22) Filed: Jan. 4, 2024

Related U.S. Application Data
(63) Continuation-in-part of application No. 17/332,718, filed on May 27, 2021.
Application No. 17/332,718 is a continuation-in-part of application No. 16/636,013, filed on Feb. 2, 2020, now Pat. No. 11,365,399.
Application No. 16/636,013 is a continuation-in-part of application No. 17/671,892, filed on Feb. 15, 2022.
Application No. 17/671,892 is a division of application No. 16/636,013, filed on Feb. 2, 2020, now Pat. No. 11,365,399.
(60) Provisional application No. 63/437,131, filed on Jan. 5, 2023.

(30) Foreign Application Priority Data
Aug. 2, 2017 (IL) 253801

(51) Int. Cl. C12N 9/04; G01N 27/327

(52) U.S. Cl. CPC C12N 9/0006; C12Y 101/05; G01N 27/3271; C07K 2319/61

(57) ABSTRACT

The present invention provides a recombinant protein including (a) lactate dehydrogenase (LDH); and (b) a minimal c-type cytochrome peptide. Further provided is a polynucleotide encoding the recombinant protein, a cell including the same, and methods of using same, such as for determining presence, concentration, or both, of an analyte in a liquid medium.

### CROSS-REFERENCE TO RELATED APPLICATIONS

This application is a continuation-in-part of U.S. patent application Ser. No. 17/332,718, titled “RECOMBINANT FLAVIN-ADENINE DINUCLEOTIDE GLUCOSE DEHYDROGENASE AND USES THEREOF”, filed on May 27, 2021, which is a continuation-in-part of U.S. patent application Ser. No. 16/636,013, titled “ELECTRONIC CHARACTERIZATION OF A DUAL CYTOCHROME-CONTAINING LACTATE DEHYDROGENASE”, filed Feb. 2, 2020, now U.S. Pat. No. 11,365,399, which is a national phase of International Patent Application No. PCT/IL2018/050863, titled “A RECOMBINANT FLAVIN-ADENINE DINUCLEOTIDE GLUCOSE DEHYDROGENASE AND USES THEREOF”, filed on Aug. 2, 2018, which claims the benefit of priority of Israel Patent Application No. 253801, titled “A RECOMBINANT FLAVIN-ADENINE DINUCLEOTIDE GLUCOSE DEHYDROGENASE AND USES THEREOF”, filed on Aug. 2, 2017.

This application is also a continuation-in-part of U.S. patent application Ser. No. 17/671,892, titled “RECOMBINANT FLAVIN-ADENINE DINUCLEOTIDE GLUCOSE DEHYDROGENASE AND USES THEREOF”, filed on Feb. 15, 2022, which is a divisional of U.S. patent application Ser. No. 16/636,013, now U.S. Pat. No. 11,365,399.

This application also claims the benefit of priority of U.S. Provisional Patent Application No. 63/437,131, titled “ELECTRONIC CHARACTERIZATION OF A DUAL CYTOCHROME-CONTAINING LACTATE DEHYDROGENASE”, filed Jan. 5, 2023.

The contents of all the above applications are incorporated herein by reference in their entirety.

### REFERENCE TO AN ELECTRONIC SEQUENCE LISTING

The contents of the electronic sequence listing (BGU-P-0136-US.xml; size: 75,452 bytes; and date of creation: Dec. 24, 2023) is herein incorporated by reference in its entirety.

### FIELD OF INVENTION

The present invention relates to recombinant proteins, methods of using the same such as for direct electron transfer in bio-electrochemical applications.

### BACKGROUND

Redox enzymes are proteins that participate in biocatalytic processes which involve electron transfer (ET). Depending on their redox potential, enzyme mediated redox reactions may be used in anodes and cathodes of biofuel cells as well as in biosensing applications. For the utilization of most redox enzymes in such devices, a mediator molecule should be used to mediate the ET between the enzyme and the electrode. The use of an external redox mediator results in a potential loss as well as in low power outputs.

Redox mediator molecules introduce two major challenges to the system: the first is having a middle point potential value that affords an efficient electron transfer from an enzyme to the electrode, which results in insufficient energy production. The other is the need for diffusion of the mediator molecule through solution towards the electrode which might cause an additional loss of energy.

### SUMMARY OF THE INVENTION

According to the first aspect, there is provided a recombinant protein, comprising (a) lactate dehydrogenase (LDH); and (b) a minimal c-type cytochrome peptide.

According to another aspect, there is provided a polynucleotide comprising a nucleic acid sequence encoding the recombinant protein of the invention.

According to another aspect, there is provided an expression vector or a plasmid comprising the polynucleotide of the invention.

According to another aspect, there is provided a transgenic or a transfected cell comprising the polynucleotide of the invention.

According to another aspect, there is provided an extract obtained or derived from the transgenic or transfected cell of the invention.

According to another aspect there is provided composition comprising the recombinant protein of the invention, and an acceptable carrier.

According to another aspect, there is provided an electrode coupled to the recombinant protein of the invention, wherein coupled is by non-covalent interactions.

According to another aspect, there is provided a device comprising the electrode of the invention.

According to another aspect, there is provided a method for determining presence, concentration, or both, of an analyte in a liquid medium, the analyte being capable of undergoing a biocatalytic oxidation or reduction reaction in the presence of an oxidizer or a reducer, respectively, the method comprising: i. providing the device of the invention; ii. contacting the device with the liquid medium; iii. measuring the electric signal generated between a cathode and an anode, the electric signal being indicative of any one of the presence of the analyte, the concentration of the analyte, and both; and vi. determining the presence, concentration, or both, of the analyte in the liquid medium based on the electric signal measured in step (iii).

In some embodiments, the recombinant protein is characterized by a Michaelis-Menten constant (K_{M}^{app}) ranging between 0.45 and 0.65 mM.

In some embodiments, the recombinant protein is characterized by a catalytic constant (k_{cat}^{app}) ranging between 10 and 15 [Sec]⁻¹.

In some embodiments, the LDH is Saccharomyces cerevisiae LDH.

In some embodiments, the recombinant protein further comprises a linker.

In some embodiments, the recombinant protein is further being bound to a porphyrin comprising a metal.

In some embodiments, the polynucleotide comprises a nucleic acid sequence set forth in SEQ ID NO: 42.

In some embodiments, the polynucleotide is operably linked to a regulatory element.

In some embodiments, the regulatory element is a T7 promoter.

In some embodiments, the transgenic or transfected cell is a prokaryotic cell.

In some embodiments, the analyte comprises lactate.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings and images in detail, it is stressed that the particulars shown are by way of example and for purposes of illustrative discussion of embodiments of the invention. In this regard, the description taken with the drawings and images makes apparent to those skilled in the art how embodiments of the invention may be practiced.

FIG. 1 includes a 3D model of Flavin-adenine dinucleotide dependent glucose dehydrogenase FAD-GDH-MCD (FGM) based on the structure of GDH, predicted by homology to formate oxidase using Swiss-model (3q9t) and on the structure of MCD from MamP crystal structure (4jj0). FAD-GDH from Burkholderia cepacia is presented with its FAD binding motif (orange, “1”) and the FAD co-factor MCD model (light orange, “2”) and the linker (grey, “3”) were cut from mamP known 3D structure and include the Heme binding motif (red, “4”) and heme molecule (pink, “5”). Heme and FAD molecules were attached to the protein model manually using PyMOL;

FIGS. 2A-2B include micrographs showing Coomassie stained sodium dodecyl sulfate-polyacrylamide gel electrophoresis (SDS-PAGE) analysis of FGM and GDH elution fractions resulting from IMAC purification. FGM and GDH catalytic sub-units are shown between 60 to 75 kDa. Band at ca. 13 kDa in both lanes correlates to FAD-GDH γ subunit (2A); and in-gel heme staining showing the presence of a heme molecule in FGM compared to its absence in GDH (2B, left panel) and anti 6×his-tag western blot verifying the full-length protein expression;

FIGS. 3A-3D include graphs showing 2,6-dichloroindophenol (DCIP) reduction assay comparing the oxidation of D-glucose by both FGM (▪) and GDH (●) (3A) and Heme activity measurements verifying the presence of a porphyrin in FGM compared to GDH. Measurements were performed in 37° C., 50 mM Tris buffer, pH=7.0 (3B); Peroxidase activity interference test; FGM was compared to Glucose oxidase (GOx) which is oxygen sensitive; No hydrogen peroxide was detected upon glucose oxidation by FGM while GOx, as a positive control, showed hydrogen peroxide production and its subsequent reduction by HRP is visible (3C); and absorbance spectrum of FGM and GDH showing peak in absorbance at 408 nm for FGM and no peak for GDH (3D);

FIGS. 4A-4C include graphs showing cyclic voltammograms of GCE/GDH and GCE/FGM with (+) and without (−) 5 mM glucose. The measurements were performed in 150 mM phosphate-citrate buffer, pH=5.0 at room temperature vs Ag/AgCl as a reference electrode at a scan rate of 5 mV s⁻¹ (4A) and 100 mV s⁻¹ (4B), and square-wave voltammetry (SWV)s of GCE/GDH and GCE/FGM. Measurements performed in 150 mM phosphate-citrate buffer, pH=5.0 vs Ag/AgCl reference electrode with 5 mV steps, amplitude 10 mV and a frequency 5 Hz (4C). Background GCE current subtracted from the signal (GCE: glassy carbon electrode);

FIGS. 5A-5B include graphs showing steady state currents from chronoamperometry measurements of GCE/GDH (●) and GCE/FGM (▪) using different glucose concentrations (5A); and Linweaver-Burk plots of electrochemical activity for both, FGM and GDH. Linear trend line equations were calculated to be y=123x+42.3 for GDH and y=10.1x+7.1 for FGM (5B);

FIG. 6 includes a map of expressed FGM. For the GDH, MCD sequence has been removed;

FIGS. 7A-7C include graphs showing Michaelis-Menten (7A) and Lineweaver-Burk (7B) plots of biochemical FAD-GDH activity for both, FGM and GDH. Linear trend line equations were calculated to be y=1.2x+6.7 for GDH and y=1.2x+7.7 for FGM and chronoamperometric measurements of glucose catalytic oxidation by FGM and GDH at an applied potential of 0.0 mV (7C);

FIGS. 8A-8C include graphs showing GCE/FGM selectivity test that was performed by adding 3.6 mM glucose followed by two sequential additions of sugars in their relevant physiological concentration−1.67 and 3.3 mM galactose -⋅-), 0.3 and 0.6 mM lactose ( [symbol] ), 2.9 and 5.8 mM maltose (red [symbol] ) and 1.67 and 3.3 mM xylose ( [symbol] ) (8A); other molecules interference was tested by adding 3.6 mM glucose followed by two additions of 0.17 mM ascorbic acid and 0.2 mM acetaminophen at an applied potential of 0.0 mV (8B); and 300 mV vs. Ag/AgC (8C);

FIG. 9 includes a 3D model of FAD-GDH from B. cepacia (green) is presented with its FAD binding motif (orange, “1”) and the FAD co-factor (blue, “2”). MCD model (cyan, “3”) and the linker (grey, “4”) were cut from MamP known 3D structure and include the heme molecule (pink, “5”). The fusion shown in this figure was manually generated using PyMOL software. ncAA possible incorporation sites were colored—proximity to FAD (red, “6”), to MCD (yellow, “7”), distant from both FAD and MCD (black, “8”);

FIGS. 10A-10B include non-limiting pyrene-azide linker structures with different lengths (10A) and 5-etramethylrhodamine (TAMRA)-azide chemical structure (10B);

FIGS. 11A-11E include FGM structural model: a 3D model of FGM was modeled by fusing the crystal structure of FAD-GDH from Burkholderia cepacia (γ-subunit in silver, α-subunit in gold, PDB ID: 6A2U) to the crystal structure of MCR2 domain from MamP crystal structure in dark green (PDB ID: 4110) (11A). Dimensions of protein's height and width were estimated using UCSF chimera software. Propargyl-L-lysine (PrK) at positions 5247 and T558 are labeled in light blue, FAD co-factor is blue, heme is dark red; Chemical structures of PrK (1), PDAz (2) and PCA (3) (11B); distance measurements between S247PrK residue and the FAD co-factor (left panel) and between the T558PrK residue and the heme domain (right panel) (11C); schematic illustration of the glassy-carbon electrode modification procedure (11D); and schematic illustration of the expected orientation of FGM-S247PDAz, FGM-T558PDAz and FGM-S247PCA on the electrode surface (11E);

FIGS. 12A-12C include protein expression verification gel micrographs and a graph: anti his-tag western blot of crude lysates from bacterial expression system with the supplementation (+) and without (−) of 2 mM PrK (12A); in-gel heme staining of FGM-S247PrK and FGM-T558PrK (12B); and a graph of DCIP FAD-GDH activity assay for glucose oxidation (12C). Curves showing the reduction of DCIP by FGM-S247PrK (square), GDH-S247PrK (triangle) and FGM-T558PrK (circle);

FIG. 13 includes a gel micrograph verification of PrK incorporation using TAMRA-Az. 6 μM FGM-S247PrK, GDH-S247PrK and FGM-T558PrK were conjugate to TAMRA-Az using click reaction. The conjugated protein was loaded into SDS-PAGE and a fluorescent image of the gel was taken. PM2700 was used as the protein size marker;

FIG. 14 includes graphs showing cyclic voltammograms in different scan-rates in the absence of glucose and peak current vs. scan rate plots. An arrow marks the relevant peak of each variant. The relation between the peak current and the scan rate was analyzed by simple linear regression and the R² is presented;

FIGS. 15A-15B include plasmid maps of pec86-pylOTS plasmid (15A) and pETDuet-FGMS247TAG expression plasmid (15B);

FIG. 16 includes images of atomic force microscopy (AFM) measurements of highly oriented pyrolytic graphite (HOPG) after incubation using different linkers. Measurement of the height of the HOPG surface after incubation with acetate buffer (left panel), FGM-S247PDAz (middle panel) and FGM-S247PCA (right panel). The square and line on the right panel indicate the area which its height was analyzed and is presented in the graphs below the respective image, arrows indicate a single protein on the HOPG surface. 200 nm scale bars are represented by white lines. Representative 3D images of the same modified surfaces are present. All images are of the same scale and measured areas of 1 μm×1 μm;

FIG. 17 includes a graph of differential pulse voltammetry (DPV) curves of EDC-NHS+PCA coupling reaction mixture (1) and click reaction mixture (2) that lacks the protein sample. FGM-S247PCA (4) and FGM-S247PDAz (3) curves were added for the ease of comparison;

FIGS. 18A-18H includes graphs of electrochemical characterization of wired FGM and GDH: DPV measurements of FGM-S247PDAz (2) and FGM-S247PCA (1) under argon in the absence of glucose (18A). Peaks are indicated by arrows; linearized multistep amperometry current decay plot of FGM-S247PDAz (circle), FGM-T558PDAz (diamond), GDH-S247PDAz (triangle) and FGM-S247PCA (square) (18B); CVs of FGM-S247PDAz, FGM-T558PDAz, GDH-S247PDAz and FGM-S247PCA in high scan rates before (1) and after (4, 5, 3 and 2, respectively) the addition of 5 mM glucose (18C); CV of heme-binding domain attached to the electrode surface before (1) and after (2) the addition of 5 mM of glucose (18D); CVs in scan rate of 10 mV/sec for FGM-S247PDAz, FGM-T558PDAz, GDH-S247PDAz and FGM-S247PCA before the addition of 5 mM glucose (1), and after the addition of 5 mM glucose (3, 5, 4 and 2, respectively) (FIG. 18E); Glucose-current calibration curves of FGM-S247PCA (circle), FGM-S247PDAz (square), FGM-T558PDAz (triangle) and GDH-S247PDAz (inverted triangle) measured using chronoamperometric detection upon application of a potential of 300 mV vs. Ag/AgCl reference electrode. The current from each glucose concentration (10 μM-10 mM) was measured in triplicates; curves of the mean currents are presented with standard deviations. Inset presents the current response of FGM-T558PDAz towards glucose in physiologically relevant glucose concentrations of sweat and tears (18F); FGM-T558PDAz response to glucose in ALU interference solution that was measured upon application of 150 mV vs. Ag/AgCl reference electrode. Black arrows indicate 0.1 mM glucose additions (18G); and the current response of FGM-T558PDAz in physiologically relevant range of glucose in the presence of interfering molecules. The current was measured in triplicates and the mean current values were used for the linear regression and are presented with error bars (18H);

FIGS. 19A-19B include graphs showing linear range of FGM-T558PDAz (19A) and FGM-S247PCA (19B) current response towards glucose;

FIG. 20 includes anti-His tag western blot analysis of FGM expressed from pTrc plasmid (1) vs. the use of a pETDuet plasmid (2);

FIG. 21 includes FGM 3D model structure with arrows indicating the ncAA incorporation sites;

FIGS. 22A-22C include gel micrographs and a graph showing ncAA incorporated FGM variants characterization: FGM variants after ‘click’ with a fluorescent marker (22A), in-gel heme staining of protein concentrated elution samples (22B) and a graph of FAD-GDH glucose oxidation activity assay (22C);

FIGS. 23A-23C include graphs and a chemical structure elucidation showing site-specific ‘wiring’ verification: Cyclic voltammetry (CV) of electrodes after incubation with a ‘clicked’ FGM and S247PrKFGM using a pyrene-azide linker (23A); FGM—blue and red curves, S247PrKFGM—green and purple curves; CV of ‘clicked’ S247PrKFGM vs. S247PrKFGM entrapped under a dialysis membrane. Entrapped enzymes: blue and red curves, ‘clicked’ enzymes: green and purple curves (23B); and pyrene-di-ethylene oxide-azide linker chemical structure (23C);

FIGS. 24A-24B include graphs showing click reaction effect on S247PrKFGM: CV of S247PrKFGM+pDAz no glucose (green), S247PrKFGM+pDAz 5 mM glucose (purple), S247PrKFGM without pDAz no glucose (blue), S247PrKFGM without pDAz 5 mM glucose (red); and chronoamperometry of S247PrKFGM without pDAz (blue), S247PrKFGM+pDAz (red) (24A) and S247PrKFGMpDAz specificity test (24B); and

FIG. 25 includes a graph showing CV of S247PrKFGM response to 5 mM glucose in different scan rates.

FIGS. 26A-26B include schemes and an illustration. (26A) A non-limiting schematic representation of the lactate dehydrogenase (LDH) constructs used in this study. (26B) An illustration of possible pathways of ET to the electrode. Either one or two electrons are transferred in the process.

FIGS. 27A-27B include images of a western blot and heme-staining analyses. (27A) Western blot of all LDH variants using anti-His antibodies. Differences in the molecular masses of the monomeric variants are evident and are in agreement with the calculated masses. (27B) Heme-staining of the different variants separated by denaturing SDS-PAGE. A dimer of LDH-CytC is also visible.

FIG. 28 includes a graph showing a decrease in 2,6-dichloroindophenol (DCIP) absorbance in the presence of each variant and 1 mM lactate. Values were normalized to the same initial DCIP absorbance. Shown are results for the following variants: LDH-CytB-CytC; LDH-CytB; LDH-CytC; LDH; and no lactate. Absorbance was measured at λ=600 nm.

FIGS. 29A-29B include graphs showing CVs of the four variants: LDH; LDH-CytC; LDH-CytB; and LDH-CytB-CytC in the absence of lactate (29A) or in the presence of 5 mM lactate (29B). The scan rate was 25 mV/sec, the pseudo-reference electrode was Ag/AgCl.

FIGS. 30A-30D include graphs showing DPVs of different lactate concentrations. DPVs obtained upon addition of varying concentrations of lactate to (30A) LDH-CytB-CytCd) and (30B) LDH-CytB drop-casted onto a SPE. Arrows indicate increasing lactate concentrations. The scan rate was 25 mV/sec. The reference electrode was pseudo-Ag/AgCl. (30C-30D) Calibration curves of oxidation peaks from the DPVs. The values in 30C were generated at the low lactate concentrations while the values in 30D were generated at higher lactate concentrations. Shown are the variants: LDH-CytB-CytC and LDH-CytB.

FIGS. 31A-31B include graphs showing lactate dependent bioelectrocatalytic reduction of oxygen. (31A) CVs measured in the absence (dashed line) or presence of 2 mM lactate (solid line), as well as in the absence of oxygen (1). LDH-CytB-CytC (2); LDH-CytB (3) The scan rate was 25 mV/s, and the reference electrode was Ag/AgCl. (31B) Calibration curves of peak reductive currents of the constructs for oxygen reduction in the presence of 0-2 mM lactate.

FIGS. 32A-32B include maps of plasmids pEC86 ccm (32A) and pET15b LDH-CytB-CytC (32B).

FIG. 33 includes a graph showing an absorbance spectra of the variants: LDH, LDH-CytC, LDH-CytB, and LDH-CytB-CytC, showing absorbance peaks at 408 and 550 nm.

FIG. 34 include a graph showing Michaelis Menten curves of biochemical activity for the variants: LDH, LDH-CytC, LDH-CytB, and LDH-CytB-CytC.

FIGS. 35A-35B include graphs showing dependence of LDH-CytB-CytC peaks on pH. (35A) DPVs in sodium acetate, pH 3.2, sodium acetate, pH 4.0, phosphate citrate, pH 5.0, and Tris-HCl, pH 6.0, showing shift in the peaks. (35B) pH-dependent oxidation potentials are shown. Scan rate is 25 mV/s, working electrodes are carbon nanotubes screen printed electrodes.

### DETAILED DESCRIPTION

According to the first aspect, there is provided a recombinant protein, comprising (a) lactate dehydrogenase (LDH); and (b) a minimal c-type cytochrome peptide.

In some embodiments, the LDH comprises a catalytic cytochrome B domain (CytB).

In some embodiments, the recombinant protein is characterized by a Michaelis-Menten constant (K_{M}^{app}) ranging between 0.4 and 0.7 mM, 0.45 and 0.7 mM, 0.5 and 0.7 mM, 0.55 and 0.7 mM, 0.4 and 0.65 mM, 0.45 and 0.6 mM, 0.45 and 0.55 mM, 0.5 and 0.65 mM, or 0.5 and 0.6 mM. Each possibility represents a separate embodiment of the invention.

In some embodiments, the recombinant protein is characterized by a catalytic constant (k_{cat}^{app}) ranging between 10 and 15 [Sec]⁻¹, 11 and 15 [Sec]⁻¹, 12 and 15 [Sec]⁻¹, 13 and 15 [Sec]⁻¹, 14 and 15 [Sec]⁻¹, 10 and 20 [Sec]⁻¹, or 5 and 15 [Sec]⁻¹. Each possibility represents a separate embodiment of the invention.

Methods and means for determining constants such as Michaelis-Menten and a catalytic constant are common and would be apparent to one of ordinary skill in the art, and are exemplified herein.

In some embodiments, the LDH of the recombinant protein of the invention is derived from Saccharomyces cerevisiae. In some embodiments, the LDH of the recombinant protein of the invention is or comprises Saccharomyces cerevisiae LDH.

In some embodiments, the recombinant protein of the invention is encoded by a polynucleotide comprising a nucleic acid sequence set forth in SEQ ID NO: 42, or a functional analog thereof, having at least 70%, 80%, 90%, 95%, or 99% homology or identity thereto, or any value and range therebetween. In some embodiments, the recombinant protein of the invention is encoded by a polynucleotide comprising a nucleic acid sequence set forth in SEQ ID NO: 42, or a functional analog thereof, having 70-100%, 80-100%, 90-100%, or 95-100% homology or identity thereto. Each possibility represents a separate embodiment of the invention.

In some embodiments, the recombinant protein of the invention is characterized by being capable of increasing bioelectrocatalytic current compared to a control protein. In some embodiments, a control is or comprises a wild-type protein. In some embodiments, a control is or comprises a recombinant protein. In some embodiments, a control does not comprise the recombinant protein of the invention. In some embodiments, a control protein comprises a wild-type LDH protein. In some embodiments, a control protein comprises a recombinant protein comprising LDH being devoid of the catalytic domain CytB. In some embodiments, a control protein comprises a recombinant protein comprising LDH and CytC. In some embodiments, a control protein comprises a recombinant protein comprising LDH and CytC and being devoid of the catalytic domain CytB.

In some embodiments, a control protein is encoded by a polynucleotide comprising a nucleic acid sequence as set forth in any one of SEQ ID Nos: 39-41.

Provided herein is a recombinant flavin mononucleotide lactate dehydrogenase (FMN-LDH) enzyme, polynucleotides sequences encoding same, useful for direct electron transfer such as in bio-electrochemical applications, including, but not limited to, lactate monitoring.

Provided herein is a recombinant flavin-adenine dinucleotide glucose dehydrogenase (FAD-GDH) enzyme, polynucleotides sequences encoding same, useful for direct electron transfer such as in bio-electrochemical applications, including, but not limited to, glucose monitoring.

The present invention is directed to a polypeptide comprising a non-canonical amino acid (ncAA). According to some embodiments, the present invention provides an electrode coupled to the polypeptide of the present invention.

The present invention is based, in part, on the surprising finding that using a site-specific incorporation of the ncAA, allows for a of a specific orientation towards the electrode. By controlling the incorporation of the ncAA and orientation, the catalytic current can be improved in response to glucose.

In some embodiments, the wiring of the polypeptides through different sites result in a significant effect on their electron transfer (ET) characteristics and their ability to communicate with an electrode. In some embodiments, the site-specific wiring of the polypeptides allows higher catalytic currents and lower onset potential, indicating highly efficient ET that is gained due to the site-specific wiring.

As provided in some embodiments of the present invention, fusing the enzyme with a minimal cytochrome domain (MCD), instead of large cytochrome, allows to shorten the enzyme-electrode distance and improve the direct electron transfer (DET) capabilities of the enzyme. As demonstrated hereinbelow under a non-limiting example, the fusion enzyme communicated with an electrode directly, without the use of a mediator molecule. Direct electron transfer between the redox enzyme and an electrode resulted in enhancement of chemical detection.

As demonstrated hereinbelow, the disclosed recombinant enzyme showed a substantially reduced redox potential, e.g., from +400 mV to 0 mV, thereby improving enzyme selectivity in various electrochemical applications including glucose sensing and monitoring as well as an electromotive force in a bio-electrochemical power device.

Before explaining further embodiments of the invention in detail, it is to be understood that the invention is not necessarily limited in its application to the details set forth in the following description or exemplified by the Examples. The invention is capable of other embodiments or of being practiced or carried out in various ways.

### Polypeptides

According to some embodiments, there is provided a recombinant polypeptide comprising the amino acid sequence: MKYLLPTAAAGLLLLAAQPAMAMIRAGATMPHRDRGPCGACHAIIQEFGSGYGSGPPGPEPK LDMNKQKISPAEVAKHNKPDDCWVVINGYVYDLTRFLPNHPGGQDVIKFNAGKDVTAIFEPL HAPNVIDKYIAPEKKLGPLQGSMPPELVCPPYAPGETKEDIARKEQLKSLLPPLDNIINLYDFEY LASQTLTKQAWAYYSSGANDEVTHRENHNAYHRIFFKPKILVDVRKVDISTDMLGSHVDVPF YVSATALCKLGNPLEGEKDVARGCGQGVTKVPQMISTLASCSPEEIIEAAPSDKQIQWYQLYV NSDRKITDDLVKNVEKLGVKALFVTVDAPSLGQREKDMKLKFSNTKAGPKAMKKTNVEESQ GASRALSKFIDPSLTWKDIEELKKKTKLPIVIKGVQRTEDVIKAAEIGVSGVVLSNHGGRQLDF SRAPIEVLAETMPILEQRNLKDKLEVFVDGGVRRGTDVLKALCLGAKGVGLGRPFLYANSCY GRNGVEKAIEILRDEIEMSMRLLGVTSIAELKPDLLDLSTLKARTVGVPNDVLYNEVYEGPTLT EFEDA (SEQ ID NO: 43), or a functional analog thereof having 80-100%, 90-100%, 95-100%, or 97-100% sequence homology or identity thereto.

According to some embodiments, there is provided a recombinant polypeptide comprising the amino acid sequence:

(SEQ ID NO: 20)
MADTDTQKADVVVVGSGVAGAIVAHQLAMAGKSVILLEAGPRMPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
XAVVYKLETGPDKRITAAVYKDKTGADHRVEGKYFVIAANGIETPKILLMSANRDFPNGVAN
SSDMVGRNLMDHPGTGVSFYANEKLWPGRGPQEMTSLIGFRDGPFRANEAAKKIHLSNMSRI
NQETQKIFKGGKLMKPEELDAQIRDRSARFVQFDCFHEILPQPENRIVPSKTATDAVGIPRPEIT
YAIDDYVKRGAVHTREVYATAAKVLGGTEVVENDEFAPNNHITGATIMGADARDSVVDKDC
RAFDHPNLFISSSSTMPTVGTVNVTLTIAALALRMSDTLKKEVEFGSGYGSGPPGPIRAGATMP
HRDRGPCGACHAIIQ,
wherein X is a non-canonical amino acid (ncAA).

According to some embodiments, there is provided a recombinant polypeptide comprising the amino acid sequence:

(SEQ ID NO: 20)
MADTDTQKADVVVVGSGVAGAIVAHQLAMAGKSVILLEAGPRMPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
SAVVYKLETGPDKRITAAVYKDKTGADHRVEGKYFVIAANGIETPKILLMSANRDFPNGVAN
SSDMVGRNLMDHPGTGVSFYANEKLWPGRGPQEMTSLIGFRDGPFRANEAAKKIHLSNMSRI
NQETQKIFKGGKLMKPEELDAQIRDRSARFVQFDCFHEILPQPENRIVPSKTATDAVGIPRPEIT
YAIDDYVKRGAVHTREVYATAAKVLGGTEVVENDEFAPNNHITGATIMGADARDSVVDKDC
RAFDHPNLFISSSSTMPTVGTVNVTLTIAALALRMSDTLKKEVEFGSGYGSGPPGPIRAGAXMP
HRDRGPCGACHAIIQ,
wherein X is a non-canonical amino acid (ncAA).

In some embodiments, the ncAA comprises Propargyl-lysine (PrK).

In some embodiments, the ncAA is covalently bound to a mediator molecule comprising polycyclic aromatic system.

In some embodiments, the ncAA is covalently bound to a mediator molecule, wherein the mediator molecule is represented by Formula I:

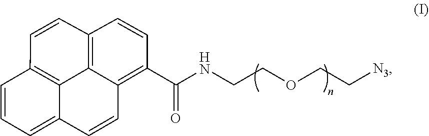

wherein n is an integer in a range from 1 to 5.

In some embodiments, n is an integer in a range from 1 to 4, 1 to 3, 2 to 5, 2 to 4, or 2 to 3, including any range therebetween. Each possibility represents a separate embodiment of the invention.

In some embodiments, n equals 3. In some embodiments, n equals 2.

As used herein, the terms “polypeptide”, “peptide” and “protein” are used interchangeably to refer to two or more amino acids linked together. The terms “polypeptide”, “peptide”, “protein”, and “amino acid sequence” as used herein refer to any compound comprising naturally occurring or synthetic amino acid polymers or amino acid-like molecules including but not limited to compounds comprising amino and/or imino molecules. No particular size is implied by use of the term “peptide”, “oligopeptide”, “polypeptide”, or “protein”. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, multiple antigenic peptide (MAP) forms, linearly-linked peptides), cyclized, branched molecules and the like, are included within the definition.

In some embodiments, the mediator molecule comprises a diethylene oxide arm or group. In some embodiments, the mediator molecule is a linker. In some embodiments, the terms “mediator molecule” and “linker: are used herein interchangeably.

In some embodiments, the diethylene oxide arm or group has a length of 2-8 Å, 3-8 Å, 4-8 Å, 5-8 Å, 6-8 Å, 6-7 Å, 4-6.5 Å, 5.5-7.5 Å, or 5.8-6.6 Å. each possibility represents a separate embodiment of the invention.

In some embodiments, the diethylene oxide arm or group has a length of not more than 5 Å, 5.2 Å, 5.4 Å, 5.6 Å, 5.8 Å, 6.0 Å, 6.1 Å, 6.2 Å, 6.3 Å, 6.4 Å, 6.6 Å, 6.8 Å, 7.0 Å, or any value and range therebetween. Each possibility represents a separate embodiment of the invention.

In some embodiments, “diethylene oxide arm or group length” as used herein, comprises total length, average length, or maximal length, of the diethylene oxide arm or group.

### Polynucleotides

According to some embodiments, the present invention provides a polynucleotide comprising a nucleic acid sequence encoding the polypeptide described herein.

In some embodiments, the polynucleotide comprises the nucleic acid sequence set forth in SEQ ID NO: 15 or SEQ ID NO: 17.

(SEQ ID NO: 15)
ATGGCGGATACGGATACCCAGAAAGCGGACGTGGTCGTGGTTGGATCCGGCGTGGCAGG
CGCAATCGTGGCTCATCAACTGGCAATGGCAGGTAAAAGCGTGATCCTGCTGGAAGCTGG
TCCGCGTATGCCGCGTTGGGAAATTGTTGAACGTTTCCGCAATCAAGTCGATAAAACCGA
CTTTATGGCACCGTATCCGAGCAGCGCATGGGCACCGCATCCGGAATATGGTCCGCCGAA
TGATTACCTGATCCTGAAAGGCGAACACAAATTTAACTCACAGTACATTCGTGCAGTGGG
CGGCACCACGTGGCATTGGGCAGCCTCGGCATGGCGCTTCATCCCGAACGATTTTAAAAT
GAAAACCGTGTATGGCGTTGGTCGTGACTGGCCGATTCAGTACGATGACATCGAACATTA
TTACCAACGCGCGGAAGAAGAACTGGGCGTGTGGGGTCCGGGCCCGGAAGAAGACCTGT
ATTCACCGCGTAAAGAACCGTACCCGATGCCGCCGCTGCCGCTGAGTTTCAATGAACAAA
CCATTAAATCCGCTCTGAACGGCTATGATCCGAAATTTCACGTGGTTACGGAACCGGTGG
CCCGTAATTCGCGCCCGTACGACGGTCGCCCGACCTGCTGTGGCAACAATAACTGCATGC
CGATTTGTCCGATCGGTGCAATGTATAACGGCATCGTCCATGTGGAAAAAGCTGAACAGG
CAGGTGCTAAACTGATTGATTAGGCGGTCGTGTACAAACTGGAAACGGGCCCGGACAAA
CGTATTACCGCAGCTGTTTATAAAGATAAAACGGGTGCGGACCATCGCGTCGAAGGCAAA
TACTTCGTGATTGCGGCCAATGGTATCGAAACCCCGAAAATTCTGCTGATGAGCGCGAAC
CGTGATTTTCCGAATGGTGTGGCCAACAGTTCCGATATGGTTGGCCGCAATCTGATGGAC
CATCCGGGCACCGGCGTGAGCTTTTATGCAAACGAAAAACTGTGGCCGGGTCGTGGTCCG
CAGGAAATGACCTCTCTGATCGGTTTCCGTGATGGCCCGTTTCGCGCGAATGAAGCAGCG
AAGAAAATTCATCTGTCAAATATGTCGCGTATCAACCAGGAAACCCAAAAAATCTTTAAA
GGCGGTAAACTGATGAAACCGGAAGAACTGGATGCGCAGATCCGTGACCGCAGTGCCCG
CTTTGTTCAATTCGATTGCTTTCACGAAATCCTGCCGCAGCCGGAAAATCGTATTGTCCCG
TCCAAAACCGCAACGGACGCAGTGGGTATTCCGCGTCCGGAAATTACGTATGCGATCGAT
GACTACGTCAAACGTGGCGCAGTGCATACGCGCGAAGTTTATGCTACCGCGGCCAAAGTG
CTGGGCGGCACCGAAGTGGTCTTCAACGATGAATTTGCGCCGAATAACCACATCACCGGT
GCCACGATTATGGGCGCGGATGCCCGTGACTCAGTGGTTGATAAAGACTGTCGCGCCTTC
GATCATCCGAACCTGTTTATTAGCAGCAGCAGCACCATGCCGACGGTTGGCACCGTTAAC
GTCACCCTGACGATTGCAGCTCTGGCACTGCGTATGTCTGATACGCTGAAAAAAGAAGTC
GAATTCGGTTCTGGTTATGGCTCTGGTCCGCCGGGTCCGATTCGTGCAGGTGCTACCATGC
CGCATCGTGATCGTGGTCCGTGCGGTGCATGTCACGCTATTATCCAGGGCAGTGGTTCCG
GCCATCACCATCACCATCACTAA.

In some embodiments, the polynucleotide comprises the nucleic acid sequence:

(SEQ ID NO: 17)
ATGGCGGATACGGATACCCAGAAAGCGGACGTGGTCGTGGTTGGATCCGGCGTGGCAGG
CGCAATCGTGGCTCATCAACTGGCAATGGCAGGTAAAAGCGTGATCCTGCTGGAAGCTGG
TCCGCGTATGCCGCGTTGGGAAATTGTTGAACGTTTCCGCAATCAAGTCGATAAAACCGA
CTTTATGGCACCGTATCCGAGCAGCGCATGGGCACCGCATCCGGAATATGGTCCGCCGAA
TGATTACCTGATCCTGAAAGGCGAACACAAATTTAACTCACAGTACATTCGTGCAGTGGG
CGGCACCACGTGGCATTGGGCAGCCTCGGCATGGCGCTTCATCCCGAACGATTTTAAAAT
GAAAACCGTGTATGGCGTTGGTCGTGACTGGCCGATTCAGTACGATGACATCGAACATTA
TTACCAACGCGCGGAAGAAGAACTGGGCGTGTGGGGTCCGGGCCCGGAAGAAGACCTGT
ATTCACCGCGTAAAGAACCGTACCCGATGCCGCCGCTGCCGCTGAGTTTCAATGAACAAA
CCATTAAATCCGCTCTGAACGGCTATGATCCGAAATTTCACGTGGTTACGGAACCGGTGG
CCCGTAATTCGCGCCCGTACGACGGTCGCCCGACCTGCTGTGGCAACAATAACTGCATGC
CGATTTGTCCGATCGGTGCAATGTATAACGGCATCGTCCATGTGGAAAAAGCTGAACAGG
CAGGTGCTAAACTGATTGATAGTGCGGTCGTGTACAAACTGGAAACGGGCCCGGACAAA
CGTATTACCGCAGCTGTTTATAAAGATAAAACGGGTGCGGACCATCGCGTCGAAGGCAAA
TACTTCGTGATTGCGGCCAATGGTATCGAAACCCCGAAAATTCTGCTGATGAGCGCGAAC
CGTGATTTTCCGAATGGTGTGGCCAACAGTTCCGATATGGTTGGCCGCAATCTGATGGAC
CATCCGGGCACCGGCGTGAGCTTTTATGCAAACGAAAAACTGTGGCCGGGTCGTGGTCCG
CAGGAAATGACCTCTCTGATCGGTTTCCGTGATGGCCCGTTTCGCGCGAATGAAGCAGCG
AAGAAAATTCATCTGTCAAATATGTCGCGTATCAACCAGGAAACCCAAAAAATCTTTAAA
GGCGGTAAACTGATGAAACCGGAAGAACTGGATGCGCAGATCCGTGACCGCAGTGCCCG
CTTTGTTCAATTCGATTGCTTTCACGAAATCCTGCCGCAGCCGGAAAATCGTATTGTCCCG
TCCAAAACCGCAACGGACGCAGTGGGTATTCCGCGTCCGGAAATTACGTATGCGATCGAT
GACTACGTCAAACGTGGCGCAGTGCATACGCGCGAAGTTTATGCTACCGCGGCCAAAGTG
CTGGGCGGCACCGAAGTGGTCTTCAACGATGAATTTGCGCCGAATAACCACATCACCGGT
GCCACGATTATGGGCGCGGATGCCCGTGACTCAGTGGTTGATAAAGACTGTCGCGCCTTC
GATCATCCGAACCTGTTTATTAGCAGCAGCAGCACCATGCCGACGGTTGGCACCGTTAAC
GTCACCCTGACGATTGCAGCTCTGGCACTGCGTATGTCTGATACGCTGAAAAAAGAAGTC
GAATTCGGTTCTGGTTATGGCTCTGGTCCGCCGGGTCCGATTCGTGCAGGTGCTTAGATGC
CGCATCGTGATCGTGGTCCGTGCGGTGCATGTCACGCTATTATCCAGGGCAGTGGTTCCG
GCCATCACCATCACCATCACTAA.

In some embodiments, the polynucleotide comprises the nucleic acid sequence:

(SEQ ID NO: 7)
TAATACGACTCACTATAGGGGAATTGTGAGCGGATAACAATTCCCCTCTAGAAATAATTT
TGTTTAACTTTAAGAAGGAGATATACCATGGGCAGCCATGGCTCACAATGACAACACCCC
GCACTCCCGCCGTACCGGCGATGCGGCCGTGACCGGTATTACGCGTCGCCAGTGGCTGCA
AGGCGCGCTGGCCCTGACCGCAGCTGGCCTGACGGGTTCCCTGGCCCTGCGCGCACTGGC
TGATGATCCGGGCACCGCACCGCTGGATACCTTTATGACGCTGAGCGAAGCTCTGACGGG
CAAAAAAGGTCTGTCTCGTGTTCTGGGCCAGCGTTTTCTGCAAGCGCTGCAAAAAGGTTC
ATTCAAAACCGCGGATTCGCTGCCGCAGCTGGCGGGCGCCCTGGCAAGCGGTTCTCTGAA
CCCGGACCAAGAAGCTCTGGCGCTGAAAATCCTGGAAGCATGGTATCTGGGCATTGTTGA
TAATGTGGTTATCACCTACGAAGAAGCCCTGATGTTTAGTGTCGTGTCCGACACGCTGGTC
ATTCCGAGCTATTGCCCGAACAAACCGGGTTTCTGGGCCGAAAAACCGATCGAACGTCAG
GCATAATGGCAGCCATCACCATCATCACCACAGCCAGGATCCGAATTCGAGCTCGGCGCG
CCTGCAGGTCGACAAGCTTGCGGCCGCATAATGCTTAAGTCGAACAGAAAGTAATCGTAT
TGTACACGGCCGCATAATCGAAATTAATACGACTCACTATAGGGGAATTGTGAGCGGATA
ACAATTCCCCATCTTAGTATATTAGTTAAGTATAAGAAGGAGATATACATATGGCAGATC
TCAATTGATGGCGGATACGGATACCCAGAAAGCGGACGTGGTCGTGGTTGGATCCGGCGT
GGCAGGCGCAATCGTGGCTCATCAACTGGCAATGGCAGGTAAAAGCGTGATCCTGCTGGA
AGCTGGTCCGCGTATGCCGCGTTGGGAAATTGTTGAACGTTTCCGCAATCAAGTCGATAA
AACCGACTTTATGGCACCGTATCCGAGCAGCGCATGGGCACCGCATCCGGAATATGGTCC
GCCGAATGATTACCTGATCCTGAAAGGCGAACACAAATTTAACTCACAGTACATTCGTGC
AGTGGGCGGCACCACGTGGCATTGGGCAGCCTCGGCATGGCGCTTCATCCCGAACGATTT
TAAAATGAAAACCGTGTATGGCGTTGGTCGTGACTGGCCGATTCAGTACGATGACATCGA
ACATTATTACCAACGCGCGGAAGAAGAACTGGGCGTGTGGGGTCCGGGCCCGGAAGAAG
ACCTGTATTCACCGCGTAAAGAACCGTACCCGATGCCGCCGCTGCCGCTGAGTTTCAATG
AACAAACCATTAAATCCGCTCTGAACGGCTATGATCCGAAATTTCACGTGGTTACGGAAC
CGGTGGCCCGTAATTCGCGCCCGTACGACGGTCGCCCGACCTGCTGTGGCAACAATAACT
GCATGCCGATTTGTCCGATCGGTGCAATGTATAACGGCATCGTCCATGTGGAAAAAGCTG
AACAGGCAGGTGCTAAACTGATTGATAGTGCGGTCGTGTACAAACTGGAAACGGGCCCG
GACAAACGTATTACCGCAGCTGTTTATAAAGATAAAACGGGTGCGGACCATCGCGTCGAA
GGCAAATACTTCGTGATTGCGGCCAATGGTATCGAAACCCCGAAAATTCTGCTGATGAGC
GCGAACCGTGATTTTCCGAATGGTGTGGCCAACAGTTCCGATATGGTTGGCCGCAATCTG
ATGGACCATCCGGGCACCGGCGTGAGCTTTTATGCAAACGAAAAACTGTGGCCGGGTCGT
GGTCCGCAGGAAATGACCTCTCTGATCGGTTTCCGTGATGGCCCGTTTCGCGCGAATGAA
GCAGCGAAGAAAATTCATCTGTCAAATATGTCGCGTATCAACCAGGAAACCCAAAAAATC
TTTAAAGGCGGTAAACTGATGAAACCGGAAGAACTGGATGCGCAGATCCGTGACCGCAG
TGCCCGCTTTGTTCAATTCGATTGCTTTCACGAAATCCTGCCGCAGCCGGAAAATCGTATT
GTCCCGTCCAAAACCGCAACGGACGCAGTGGGTATTCCGCGTCCGGAAATTACGTATGCG
ATCGATGACTACGTCAAACGTGGCGCAGTGCATACGCGCGAAGTTTATGCTACCGCGGCC
AAAGTGCTGGGCGGCACCGAAGTGGTCTTCAACGATGAATTTGCGCCGAATAACCACATC
ACCGGTGCCACGATTATGGGCGCGGATGCCCGTGACTCAGTGGTTGATAAAGACTGTCGC
GCCTTCGATCATCCGAACCTGTTTATTAGCAGCAGCAGCACCATGCCGACGGTTGGCACC
GTTAACGTCACCCTGACGATTGCAGCTCTGGCACTGCGTATGTCTGATACGCTGAAAAAA
GAAGTCGAATTCGGTTCTGGTTATGGCTCTGGTCCGCCGGGTCCGATTCGTGCAGGTGCTA
CCATGCCGCATCGTGATCGTGGTCCGTGCGGTGCATGTCACGCTATTATCCAGGGCAGTG
GTTCCGGCCATCACCATCACCATCACTAAAAGCGATATCGGCCGGCCACGCGATCGCTGA
CGTCGGTACCCTCGAGTCTGGTAAAGAAACCGCTGCTGCGAAATTTGAACGCCAGCACAT
GGACTCGTCTACTAGCGCAGCTTAATTAACCTAGGCTGCTGCCACCGCTGAGCAATAACT
AGCATAACCCCTTGGGGCCTCTAAACGGGTCTTG.

The terms “polynucleotide” and “nucleic acid” as used interchangeably herein, refer to polymers of nucleotides of any length, and include DNA and RNA, or any combination thereof.

Polynucleotides encoding polypeptides may be obtained from any source including, but not limited to, a cDNA library prepared from tissue believed to possess the polypeptide mRNA and to express it at a detectable level. Accordingly, polynucleotides encoding a polypeptide can be conveniently obtained from a cDNA library prepared from human tissue. The polypeptide-encoding gene may also be obtained from a genomic library or by known synthetic procedures (e.g., automated nucleic acid synthesis).

In some embodiments, the polynucleotide is codon optimized to facilitate or increase translation efficiency in a host cell. In some embodiments, the polynucleotide is codon optimized for expression in a microorganism cell. Methods for codon optimization are common and would be apparent to one of ordinary skill in the art, as well as codon preference of various types of cells, e.g., of E. coli.

In some embodiments, there is provided a polynucleotide comprising: (a) a first regulatory element operably linked to the alpha catalytic subunit of the FGM expression gene; and (b) a second regulatory element operably linked to the gamma catalytic subunit of the FGM expression gene.

In some embodiments, the regulatory element is a promoter.

In some embodiments, the first regulatory element and the second regulatory element are identical. In some embodiments, the first regulatory element and the second regulatory element is a bacteriophage. In some embodiments, the first regulatory element and the second regulatory element is a T7 phage.

In some embodiments, the polynucleotide is a DNA molecule.

According to some embodiments, there is provided a vector or a plasmid comprising the polynucleotide of the invention.

In some embodiments, the expression vector or plasmid further comprises a nucleic acid sequence encoding a gamma subunit of an FAD-GDH.

In some embodiments, each of the polynucleotide encoding the polypeptide of the invention, and the nucleic acid sequence encoding the gamma subunit of an FAD-GDH are operably linked to a separate regulatory element.

In some embodiments, the expression vector further comprises a nucleic acid sequence encoding a gamma subunit of an FAD-GDH, wherein each of the polynucleotide encoding the polypeptide of the present invention, and the nucleic acid sequence encoding the gamma subunit of an FAD-GDH are operably linked to a separate regulatory element. In some embodiments, the regulatory element is a promoter. In some embodiments, the promoter is a T7 promoter.

According to some embodiments, there is provided a transgenic or a transfected cell comprising: a) the polypeptide disclosed herein; b) the polynucleotide disclosed herein; c) the expression vector or the plasmid disclosed herein; or d) any combination of (a) to (c).

In some embodiments, the cell is a prokaryotic cell. In one embodiment, the cell is a bacterial cell.

According to some embodiments, there is provided an extract obtained or derived from the cell disclosed herein. In some embodiments, the extract comprises: a) the polypeptide disclosed herein; b) the polynucleotide disclosed herein; c) the expression vector or the plasmid disclosed herein; d) any combination of (a) to (c).

According to some embodiments, there is provided a composition comprising: a) the polypeptide disclosed herein; b) the polynucleotide disclosed herein; c) the expression vector or the plasmid disclosed herein; d) the cell disclosed herein; e) the extract disclosed herein; f) any combination of (a) to (e); and g) and an acceptable carrier.

According to some embodiments, there is provided an electrode coupled to the polypeptide described herein. In some embodiments, coupled is by non-covalent interactions. In some embodiments, non-covalent interactions refer to pi-pi stacking interactions between the pyrene groups of the mediator molecule represented by Formula I and the electrode surface. As used herein “pi-pi stacking” refers to attractive, non-covalent interactions between aromatic rings.

According to some embodiments, there is provided a device comprising the electrode described hereinabove.

According to some embodiments, there is provided a method for determining an analyte in a liquid medium, the analyte being capable to undergo a biocatalytic oxidation or reduction reaction in the presence of an oxidizer or a reducer, respectively, the method comprising: (i) providing the device described hereinabove; (ii) contacting the device with the liquid medium; (iii) measuring the electric signal generated between the cathode and the anode, the electric signal being indicative of the presence and/or the concentration of the analyte; and (iv) determining the analyte based on the electric signal.

In some embodiments, the analyte comprises lactate.

In some embodiments, the analyte comprises glucose.

According to some embodiments, there is provided a method for transferring an electron to an electrode, comprising coupling the polypeptide described herein to an electrode, thereby transferring an electron to the electrode.

According to some embodiments, there is provided a method for quantifying the amount of a reporter in a sample having a first detectable range of light absorbance in an oxidized state a second range of light absorbance in a non-oxidized state, comprising: (a) contacting polypeptide described herein with the reporter in a non-oxidized state; and (b) measuring the amount of the reporter in an oxidized state, thereby quantifying the amount of a reporter in a sample.

In some embodiments, the first detectable range of light absorbance is detectable in visible light and the second range of light absorbance is non-detectable in visible light. In some embodiments, the reporter is 2,6-Dichloroindophenol. In some embodiments, the 2,6-Dichloroindophenol is coupled to glucose.

According to some embodiments, there is provided a method for expression of the polypeptide described herein, comprising expressing a first subunit from a first regulatory element and expressing a second subunit from a second regulatory element, thereby expressing the polypeptide described herein.

In some embodiments, a first subunit is an alpha catalytic subunit of the FGM. In some embodiments, a second subunit is a gamma catalytic subunit of the FGM.

In some embodiments, the regulatory element is a promoter.

In some embodiments, the first regulatory element and the second regulatory element are identical. In some embodiments, the first regulatory element and the second regulatory element is a bacteriophage. In some embodiments, the first regulatory element and the second regulatory element is a T7 phage.

The present invention provides, in some embodiments, a recombinant protein comprising: (a) an alpha subunit of an FAD-GDH; and (b) a minimal cytochrome peptide.

In one embodiment, the alpha subunit of the FAD-GDH is derived or recovered from a prokaryotic cell. In one embodiment, the alpha subunit of the FAD-GDH is derived or recovered from a bacterial cell. In one embodiment, the alpha subunit of the FAD-GDH is Burkholderia cepacian alpha subunit of FAD-GDH. In one embodiment, the alpha subunit of the FAD-GDH of the present invention is derived from a thermostable enzyme, an oxygen independent enzyme, or both.

The term “thermostable enzyme” refers to an enzyme that is relatively stable to heat. The thermostable enzymes can withstand the high temperature incubation used to remove the modifier groups, typically, but not exclusively, greater than 50° C., without suffering an irreversible loss of activity.

In one embodiment, the recombinant protein further comprises the gamma subunit of an FAD-GDH. In one embodiment, the invention provides a composition comprising or consisting the recombinant protein with or without the gamma subunit of an FAD-GDH. In one embodiment, the invention provides a composition comprising or consisting the recombinant protein and the gamma subunit of an FAD-GDH. In one embodiment, the invention provides a composition comprising or consisting at least two different proteins: (a) the recombinant protein; and (b) the gamma subunit of an FAD-GDH. In one embodiment, the at least two different proteins are unbound. In some embodiments, the gamma subunit is from the same FAD-GDH as the alpha subunit. In some embodiments, the gamma subunit is from a different FAD-GDH as the alpha subunit. In some embodiments, the gamma subunit is from the same or different FAD-GDH as the alpha subunit.

In one embodiment, the recombinant protein further comprises a minimal cytochrome peptide. In one embodiment, the minimal cytochrome peptide is a natural peptide. In one embodiment, the minimal cytochrome peptide comprises a non-natural peptide.

In one embodiment, the recombinant protein is devoid of the gamma subunit of the FAD-GDH. In one embodiment, the minimal cytochrome peptide comprises a c-type cytochrome domain. In one embodiment, the minimal cytochrome peptide does not comprise a b-type cytochrome domain. In one embodiment, the minimal cytochrome peptide comprises a c-type cytochrome domain MCR-2 from a MamP protein. In one embodiment, the minimal cytochrome peptide comprises a magnetotactic bacterius minimal cytochrome peptide. In one embodiment, the minimal cytochrome peptide is a magnetotactic bacterius minimal cytochrome peptide.

In one embodiment, the minimal cytochrome peptide is a peptide comprising or consisting of 11 to 30 amino acids. In one embodiment, the minimal cytochrome peptide is a peptide comprising or consisting of 11 to 24 amino acids. In some embodiments, the minimal domain is a naturally occurring cytochrome. In some embodiments, the minimal domain is a synthetic cytochrome.

In one embodiment, the minimal cytochrome peptide is a cytochrome peptide (e.g., c-type cytochrome) comprising or consisting of 11, 12, 13, 14, 15, 16, 17, 18, 19, 20, 21, 22, 23, 24, 25, 26, 27, 28, 29, or 30 amino acids, including any range therebetween. In some embodiments, the peptide comprises cytochrome functionality. In some embodiments, the peptide comprises ET functionality.

In one embodiment, the minimal cytochrome peptide is linked to the amino terminus of the alpha subunit of an FAD-GDH. In one embodiment, the minimal cytochrome peptide is linked to the carboxy terminus of the alpha subunit of an FAD-GDH. In one embodiment, the minimal cytochrome peptide is linked to the amino terminus of the gamma subunit of an FAD-GDH. In one embodiment, the minimal cytochrome peptide is linked to the carboxy terminus of the gamma subunit of an FAD-GDH.

In one embodiment, the minimal cytochrome peptide is linked to the subunit of an FAD-GDH directly or indirectly. In one embodiment, the minimal cytochrome peptide is linked to the carboxy terminus of the subunit of an FAD-GDH directly or indirectly.

In one embodiment, the recombinant protein, further comprises a linker. In one embodiment, the recombinant protein, further comprises a peptide linker. In one embodiment, the minimal cytochrome peptide is linked to the carboxy terminus or the amino terminus of the alpha subunit of an FAD-GDH via a peptide linker. In one embodiment, the minimal cytochrome peptide is linked to the carboxy terminus or the amino terminus of the gamma subunit of an FAD-GDH via a peptide linker. In one embodiment, the minimal cytochrome peptide is linked to the carboxy terminus or the amino terminus of the LDH via a peptide linker. In one embodiment, LDH is or comprises flavin mononucleotide (FMN)-LDH. In one embodiment, the minimal cytochrome peptide is linked to the carboxy terminus or the amino terminus of FMN-LDH via a peptide linker.

In one embodiment, the minimal cytochrome peptide is linked to the amino or carboxy terminus of the subunit via a linker comprising or consisting 5 to 20 amino acids. In one embodiment, the minimal cytochrome peptide is linked to the amino or carboxy terminus of the subunit via a linker comprising or consisting 8 to 18 amino acids. In one embodiment, the minimal cytochrome peptide is linked to the amino or carboxy terminus of the subunit via a linker comprising or consisting 12 to 15 amino acids. In one embodiment, the minimal cytochrome peptide is linked to the amino or carboxy terminus of the subunit via a linker comprising or consisting 5 to 15 amino acids. In some embodiments, the linker is between 1 and 10, 1 and 9, 1 and 8, 1 and 7, 2 and 10, 2 and 9, 2 and 8, 2 and 7, 3 and 10, 3 and 9, 2 and 8 or 2 and 7 amino acids in length.

In one embodiment, the minimal cytochrome peptide is linked to the amino or carboxy terminus of the LDH via a linker comprising or consisting 5 to 20 amino acids. In one embodiment, the minimal cytochrome peptide is linked to the amino or carboxy terminus of the LDH via a linker comprising or consisting 8 to 18 amino acids. In one embodiment, the minimal cytochrome peptide is linked to the amino or carboxy terminus of the LDH via a linker comprising or consisting 12 to 15 amino acids. In one embodiment, the minimal cytochrome peptide is linked to the amino or carboxy terminus of the LDH via a linker comprising or consisting 5 to 15 amino acids.

In some embodiments, the linker comprises 30% to 60% glycine. In some embodiments, the linker comprises 30% to 60% serine. In some embodiments, the linker is hydrophilic. In some embodiments, the linker does not cause steric hinderance. In some embodiments, the linker is a flexible linker. In some embodiments, the linker does not interfere with maturation of the porphyrin binding MCD. In some embodiments, the linker does not interfere with enzymatic activity of the other subunits. In some embodiments, the linker is not so short that the other subunit interferes with maturation of the porphyrin binding MCD. In some embodiments, the linker retains the subunits in close enough proximity to allow electron transfer. In some embodiments, the linker has a length of up to 20 Å, up to 25 Å, or up to 30 Å. In some embodiments, the linker has a length of greater than or equal to 5 Å, 10 Å, 12 Å, 15 Å or 17 Å. Non-limiting exemplary linker is a peptide comprising GSGYGSG. In some embodiments, the linker comprises or consists of SEQ ID NO: 24. In some embodiments, the linker comprises or consists a non-peptide backbone.

In one embodiment, the linker is encoded by a DNA sequence comprising or consisting the nucleotide sequence: GAATTCGGTTCTGGTTATGGCTCTGGTCCGCCGGGTCCG (SEQ ID NO: 4). It will be understood by a skilled artisan that synonymous substitutions may be made to this sequence.

In one embodiment, the linker is encoded by a DNA sequence comprising or consisting of a nucleotide sequence synonymous with SEQ ID NO: 4).

Without being bound by any particular mechanism it is assumed that a short linker containing glycine renders the linker with a desired flexibility. Further, and without being bound by any particular mechanism it is assumed that a short linker containing serine renders the linker with a desired hydrophilicity.

Further, a shorter linker (e.g. shorter than to 5 Å) could prevent proper maturation of the porphyrin binding MCD (due to its close proximity to GDH); on the other hand, a longer linker (e.g., longer than 30 Å) could prevent efficient ET between the two domains.

In one embodiment, the recombinant protein further comprises a short tag peptide (3 to 20 amino acids long). In one embodiment, the short tag peptide is his tag. In some embodiments, the tag is a 6×his tag. Protein tags are well known in the art and any tag that does not interfere with the function (redox and ET) of the recombinant protein may be used. In some embodiments, the short tag is between 1 and 30, 1 and 25, 1 and 20, 1 and 15, 1 and 10, 2 and 30, 2 and 25, 2 and 20, 2 and 25, 2 and 10, 3 and 30, 3 and 25, 3 and 20, 3 and 15 or 3 and 10 amino acids in length. Each possibility represents a separate embodiment of the invention.

In one embodiment, the recombinant protein has a molecular weight in the range of 58 to 75 kDa. In one embodiment, the recombinant protein has a molecular weight in the range of 60 to 70 kDa. In one embodiment, the recombinant protein has a molecular weight in the range of 63 to 65 kDa. In one embodiment, the recombinant protein has a molecular weight in the range of 62 to 68 kDa. In one embodiment, the recombinant protein has a molecular weight in the range of 63 to 65 kDa.

In some embodiments, the amino acid sequence of the recombinant protein comprises or consists of the following sequence:

(SEQ ID NO: 8, shortened)
MADTDTQKADVVVVGSGVAGAIVAHQLAMAGKSVILLEAGPRMPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
SAVVYKLETGPDKRITAAVYKDKTGADHRVEGKYFVIAANGIETPKILLMSANRDFPNGVAN
SSDMVGRNLMDHPGTGVSFYANEKLWPGRGPQEMTSLIGFRDGPFRANEAAKKIHLSNMSRI
NQETQKIFKGGKLMKPEELDAQIRDRSARFVQFDCFHEILPQPENRIVPSKTATDAVGIPRPEIT
YAIDDYVKRGAVHTREVYATAAKVLGGTEVVFNDEFAPNNHITGATIMGADARDSVVDKDC
RAFDHPNLFISSSSTMPTVGTVNVTLTIAAL.

Herein throughout, in some embodiments, by “shortened” or “partial” it is meant to refer to without e.g., tag peptide, and/or a linker.

In some embodiments, the amino acid sequence of the recombinant protein comprises or consists of the following sequence:

(SEQ ID NO: 9, full)
MADTDTQKADVVVVGSGVAGAIVAHQLAMAGKSVILLEAGPRMPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
SAVVYKLETGPDKRITAAVYKDKTGADHRVEGKYFVIAANGIETPKILLMSANRDFPNGVAN
SSDMVGRNLMDHPGTGVSFYANEKLWPGRGPQEMTSLIGFRDGPFRANEAAKKIHLSNMSRI
NQETQKIFKGGKLMKPEELDAQIRDRSARFVQFDCFHEILPQPENRIVPSKTATDAVGIPRPEIT
YAIDDYVKRGAVHTREVYATAAKVLGGTEVVFNDEFAPNNHITGATIMGADARDSVVDKDC
RAFDHPNLFISSSSTMPTVGTVNVTLTIAALALRMSDTLKKEVIRAGATMPHRDRGPCGACHA
IIQ.

In some embodiments, the amino acid sequence of the recombinant protein comprises or consists of the following sequence:

(SEQ ID NO: 10, full)
MADTDTQKADVVVVGSGVAGAIVAHQLAMAGKSVILLEAGPRMPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
SAVVYKLETGPDKRITAAVYKDKTGADHRVEGKYFVIAANGIETPKILLMSANRDFPNGVAN
SSDMVGRNLMDHPGTGVSFYANEKLWPGRGPQEMTSLIGFRDGPFRANEAAKKIHLSNMSRI
NQETQKIFKGGKLMKPEELDAQIRDRSARFVQFDCFHEILPQPENRIVPSKTATDAVGIPRPEIT
YAIDDYVKRGAVHTREVYATAAKVLGGTEVVFNDEFAPNNHITGATIMGADARDSVVDKDC
RAFDHPNLFISSSSTMPTVGTVNVTLTIAALALRMSDTLKKEVEFGSGYGSGPPGPIRAGATMP
HRDRGPCGACHAIIQGSGSGHHHHHH.

In some embodiments, the amino acid sequence of the recombinant protein comprises or consists of the following sequence:

(SEQ ID NO: 11, partial)
MADTDTQKADVVVVGSGVAGAIVAHQLAMAGKSVILLEAGPRMPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
SAVVYKLETGPDKRITAAVYKDKTGADHRVEGKYFVIAANGIETPKILLMSANRDFPNGVAN
SSDMVGRNLMDHPGTGVSFYANEKLWPGRGPQEMTSLIGFRDGPFRANEAAKKIHLSNMSRI
NQETQKIFKGGKLMKPEELDAQIRDRSARFVQFDCFHEILPQPENRIVPSKTATDAVGIPRPEIT
YAIDDYVKRGAVHTREVYATAAKVLGGTEVVFNDEFAPNNHITGATIMGADARDSVVDKDC
RAFDHPNLFISSSSTMPTVGTVNVTLTIAALALRMSDTLKKEVEFGSGYGSGPPGPIRAGATMP
HRDRGPCGACHAIIQ.

An electrode

In some embodiments, there is provided an electrode carrying or coupled to a recombinant protein comprising A, B, C, and D, wherein: A is a cofactor of a redox enzyme; B is a redox enzyme; C is a linker moiety; and D is an electron transfer (ET) domain that is configured to transfer electrons between the electrode and A. In some embodiment the ET comprises a cytochrome.

In one embodiment, the A, B, C, and D are linked to each other under the following order: A-B-C-D.

In some embodiments, the cofactor, when not bound to a linker moiety, comprises at least one pair of hydroxyl groups.

In one embodiment, there is provided a device comprising the electrode.

In one embodiment, the electrode comprises a material selected from, without being limited thereto, graphite and glassy carbon electrode (GCE).

In one embodiment, the electrode is made of or coated by an electrically conducting substance, such as, without being limited thereto gold, platinum, silver, conducting glass such as indium tin oxide (ITO).

In one embodiment, by “chemically attach to” it is meant to refer to being attached via a covalent bond. As used herein, the term “coupled” refers to a physical attachment, such that the two are bonded together. In some embodiments, the bond is a covalent bond. In some embodiments, the bond is a synthetic bond. In some embodiments, the bond is a chemical bond.

In another embodiment, by “chemically attach to” it is meant to refer to being attached to (“wiring”) the electrode via a synthetic linker (also referred “electrode linker” or “mediator molecule” or “mediator”) being configured to link the recombinant protein to an electrode.

In one embodiment, the wiring can be obtained by a non-specific process, by using a chemical modification and conductive matrices such as, without limitation, graphene oxide and multi-walled carbon nanotubes.

In one embodiment, the wiring is a site-specific wiring, performed e.g., by inserting at least one non-canonical amino acid in a desired site of one of the groups (A to D) that, optionally, covalently links to a moiety that binds to an electrode as described herein.

Without being bound by any particular mechanism, it is assumed that the recombinant protein disclosed herein (e.g., in the form of A-B-C-D) allows direct electron transfer (DET) via a domain that affords DET, resulting in a “built in” redox mediator.

In some embodiments, in order to achieve an efficient DET, the distance between the enzyme's active site and the electrode is as short as a few Ångstroms (e.g., 1 to 20 Å). In some embodiments, the distance is between 0-20, 0-19, 0-18, 0-17, 0-16, 0-15, 0-14, 0-13, 0-12, 0-11, 0-10, 1-20, 1-19, 1-18, 1-17, 1-16, 1-15, 1-14, 1-13, 1-12, 1-11, 1-10, 2-20, 2-19, 2-18, 2-17, 2-16, 2-15, 2-14, 2-13, 2-12, 2-11, 3-10, 3-20, 3-19, 3-18, 3-17, 3-16, 3-15, 3-14, 3-13, 3-12, 3-11, or 3-10 Å. Each possibility represents a separate embodiment of the invention.

In some embodiments, when linked to the enzyme, the ET domain is minimal so as not to introduce additional insulation to the system by a complex proteinaceous matrix. In some embodiments, the ET domain is linked by a flexible linker. As described herein, the flexibility allows avoiding interruption of the catalytic redox activity. In some embodiments, the minimal domain is not more than 10, 12, 15, 17, 20, 22, 25, 27, 30, 32, 35, 37, 40, 42, 45, 47 or 50 amino acids. Each possibility represents a separate embodiment of the invention.

Embodiments of “minimal domain” are described hereinabove e.g., for the c-type cytochrome.

In some embodiments, the cofactor is selected from, without being limited thereto, FMN, FAD, NAD+, and NADP+.

In one embodiment, the redox enzyme refers to an enzyme that can catalyze a redox reaction.

In one embodiment, the redox enzyme may be selected from, without being limited thereto, oxidase, dehydrogenase, and malic enzyme (e.g., malate dehydrogenase). In some embodiments, the redox enzyme is selected from an oxidase, a dehydrogenase, a reductase, a peroxidase, a glyoxalase, a hydroxylase and a malic enzyme. In some embodiments, the redox enzyme is sugar dehydrogenase. In some embodiments, the sugar is glucose. In some embodiments, enzyme is alcohol dehydrogenase.

In one embodiment, the redox enzyme in the immobilized group is characterized by a redox potential of less than 50 mV. In one embodiment, the redox enzyme in the immobilized group is characterized by a redox potential of 50 mV, 40 mV, 30 mV, 20 mV, 10 mV, 0 V, −10 mV, −20 mV, −30 mV, −40 mV, −50 mV, −60 mV, −70 mV, −80 mV, −90 mV, −100 mV, −110 mV, −120 mV, −130 mV, −140 mV, −150 mV, −160 mV, −170 mV, −180 mV, −190 mV, or −200 mV (induced potential vs. Ag/AgCl) including any value and range therebetween.

In one embodiment, the dehydrogenase is selected from, without being limited thereto, alcohol dehydrogenase, glutamic acid dehydrogenase, cholesterol dehydrogenase, aldehyde dehydrogenase, glucose dehydrogenase, fructose dehydrogenase, sorbitol dehydrogenase, lactate dehydrogenase, and glycerol dehydrogenase.

In one embodiment, the linker moiety comprises a peptide. In one embodiment, the peptide comprises serine. In one embodiment, the linker moiety comprises a short peptide e.g. having 5 to 20 amino acids, or 5 to 15 amino acids, e.g., 5, 6, 7, 8, 9, 10, 11, 12, 13, 14, or 15 amino acids, including any range therebetween. In some embodiments, the linker is selected from any embodiments of a linker described hereinabove. In some embodiments, the linker moiety is a flexible linker. In some embodiments, the linker moiety is hydrophilic. In some embodiments, the linker moiety is synthetic. In some embodiments, the linker moiety is not from cellobiose dehydrogenase. In some embodiments, the linker moiety is not from pyranose dehydrogenase. In some embodiments, the linker moiety does not interfere with enzyme function and/or DET.

A non-limiting example of a linker is a peptide comprising GSGYGSG.

In one embodiment, the linker is characterized by a length of: 5 to 40, or 20 to 30 Å, e.g., 5, 10, 15, 20, 25, 30, 35, or 40 Å, including any value and range therebetween.

In one embodiment, the electron transfer domain comprises a cytochrome, e.g., MCD.

Embodiments of the cytochrome are described hereinabove. In some embodiments, the electron transfer domain comprises a cytochrome c. In some embodiments, the electron transfer domain does not comprise a cytochrome b. In some embodiments, the recombinant protein is not a naturally occurring protein.

In one embodiment, the device is a biosensor.

As used herein and in the art, biosensors are analytical devices that combine a biological material (e.g., tissues, microorganisms, enzymes, antibodies, nucleic acids etc.) or a biologically-derived material with a physicochemical transducer or transducing microsystem.

In one embodiment, the device comprises or is configured to attach to an electronic circuitry for energizing the electrode and measuring the response.

In some embodiments, the biosensor is for measuring the concentration of a sugar in a medium.

In some embodiments, the biosensor is for measuring the concentration of glucose in a medium. In some embodiments, the biosensor is for measuring the concentration of alcohol in a medium.

In some embodiments the medium is a bodily fluid. In some embodiments, the bodily fluid is selected from at least one of: blood, serum, gastric fluid, intestinal fluid, saliva, bile, tumor fluid, urine, breast milk, interstitial fluid, and stool. In some embodiments, the bodily fluid is or comprises sweat.

In some embodiments, the biosensor is capable of measuring sweat lactate without dilution or sample processing, which can evaluate the current status of lactate in a subject in need thereof.

In some embodiments, the biosensor is capable of measuring whole blood, serum or plasma glucose without dilution or sample processing, which can evaluate the current status of glucose or an individual suffering from diabetes. In some embodiments, the bodily fluid is undiluted. In some embodiments, the bodily fluid is diluted with a buffer.

In some embodiments, the biosensor comprises an anode compartment and a cathode compartment, the compartments being in fluid communication, wherein the anode compartment comprises an anode electrode and a substrate; and wherein the cathode compartment comprises a cathode electrode, the anode electrode and cathode electrode in electrical communication.

In some embodiments, the electrode disclosed herein is the anode.

According to an aspect, the present invention provides a method for determining an analyte in a liquid medium, the analyte being capable to undergo a biocatalytic oxidation or reduction reaction in the presence of an oxidizer or a reducer, respectively, the method comprising: (i) providing the disclosed device in an embodiment thereof; (ii) contacting the device with the liquid medium; (iii) measuring the electric signal generated between the cathode and the anode, the electric signal being indicative of the presence and/or the concentration of the analyte; and (iv) determining the analyte based on the electric signal.

In some embodiments, the method is for determining the presence of the analyte in the medium. In some embodiments, the method is for determining the concentration of the analyte in the medium. When the liquid medium is, for example, a body fluid e.g. blood, lymph fluid or cerebro-spinal fluid, and the method may be carried out in an invasive manner, the method comprises inserting the biosensor into the body and bringing it into contact with the body fluid and determining the analyte in the body fluid within the body. Alternatively, body fluids or any other analyte may be tested non-invasively, and in such cases the method may comprise adding a buffer to the fluid. In some embodiments, the buffer has pH 4 to 8. In some embodiments, the buffer has pH 5 (±1).

In some embodiments, the contacting is in vivo. In some embodiments, the contacting is ex vivo. In some embodiments, a detected electrical signal indicates the analyte is present. In some embodiments, the greater the electrical signal the greater the concentration of analyte. In some embodiments, the electrical signal is compared to a predetermined standard that indicates the concentration of the analyte based on the electrical signal.

Non-limiting examples of analytes are sugar molecules e.g., galactose, lactose, maltose and xylose glucose, fructose, maltose; lactate; bilirubin; alcohols or amino acids.

In some embodiments, provided herein is a method for transferring an electron to an electrode, comprising coupling the disclosed recombinant protein in an embodiment thereof to an electrode, thereby transferring an electron to an electrode. In one embodiment, coupling is in the absence of a mediator molecule.

In some embodiments, the amino acid sequence of the recombinant protein comprises or consists of the following sequence:

(SEQ ID NO: 12, full)
MADTDTQKADVVVVGSGVAGAIVAHQLAMAGKSVILLEAGPX1MPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
X2AVVYKLETGPDKRITAAVYKDKTGADHRVEGKYFVIAANGIETPKILLMSANRDFPNGVA
NSSDMVGRNLMDHPGTGVSFYANEKLWPGRGPQEMTSLIGFRDGPFRANEAAKKIHLSNMS
RINQETQKIFKGGKLMKPEELDAQIRX3RSARFVQFDCFHEILPQPENRIVPSKTATDAVGIPRP
EITYAIDDYVKRGAVHTREVYATAAKVLGGTEVVFNDEFAPNNHITGATIMGADARDSVVDK
DCRAFDHPNLFISSSSTMPTVGTVNVTLTIAALALRMSDTLKKEVEFGSGYGSGPPGPIRAGA
X4MX5HRDRGPCGACHAIIQGSGSGHHHHHH.

In some embodiments, the amino acid sequence of the recombinant protein comprises or consists of the following sequence:

(SEQ ID NO: 13, partial)
MADTDTQKADVVVVGSGVAGAIVAHQLAMAGKSVILLEAGPX1MPRWEIVERFRNQVDKTD
FMAPYPSSAWAPHPEYGPPNDYLILKGEHKFNSQYIRAVGGTTWHWAASAWRFIPNDFKMK
TVYGVGRDWPIQYDDIEHYYQRAEEELGVWGPGPEEDLYSPRKEPYPMPPLPLSFNEQTIKSA
LNGYDPKFHVVTEPVARNSRPYDGRPTCCGNNNCMPICPIGAMYNGIVHVEKAEQAGAKLID
X2AVVYKLETGPDKRITAAVYKDKTGADHRVEGKYFVIAANGIETPKILLMSANRDFPNGVA
NSSDMVGRNLMDHPGTGVSFYANEKLWPGRGPQEMTSLIGFRDGPFRANEAAKKIHLSNMS
RINQETQKIFKGGKLMKPEELDAQIRX3RSARFVQFDCFHEILPQPENRIVPSKTATDAVGIPRP
EITYAIDDYVKRGAVHTREVYATAAKVLGGTEVVFNDEFAPNNHITGATIMGADARDSVVDK
DCRAFDHPNLFISSSSTMPTVGTVNVTLTIAALALRMSDTLKKEVEFGSGYGSGPPGPIRAGA
X4MX5HRDRGPCGACHAIIQ.

In some embodiments, X1 is R. In some embodiments, X2 is S. In some embodiments, X3 is D. In some embodiments, X4 is T. In some embodiments, X5 is P.

In some embodiments, one or more amino acids selected from X1 to X5 comprise at least one non-canonical amino acid (ncAA) residue.

Exemplary embodiments of SEQ ID NO: 12-13 are presented in the Examples section below (e.g., SEQ ID NOs: 19-23).

The term “non-canonical amino acid residue” refers to amino acid residues in D- or L-form that are not among the 20 canonical amino acids generally incorporated into naturally occurring proteins, for example, β-amino acids, homoamino acids, cyclic amino acids and amino acids with derivatized side chains.

Non-canonical amino acid residues may be incorporated into a peptide within the scope of the invention by employing known techniques of protein engineering that use recombinantly expressing cells.

In some embodiments one or more from X1 to X5 are present in proximity to the protein domain selected from, without being limited thereto: FAD binding domain, or MCD. In some embodiments one or more from X1 to X5 are present in a site that is distant from either FAD domain or MCD.

In some embodiments, by “proximity” it is meant to refer to a distance of 10 to 25 Å, e.g., 10, 15, 20, or 25 Å, including any value and range therebetween.

In some embodiments, by “distant” it is meant to refer to a distance of 30 to 100 Å, e.g., 10, 15, 20, 25, 30, 35, 40, 45, 50, 55, 60, 65, 70, 75, 80, 85, 90, 95, or 100 Å.

In some embodiments, the ncAA is a clickable ncAA.

By “clickable ncAA” it is meant to refer to ncAA attachable to another group or moiety by biorthogonal chemical mechanism.

In exemplary embodiments, the ncAA comprises Propargyl-lysine (PrK).

In some embodiments, the ncAA has attached thereto an electrode linker (mediator) configured to couple the recombinant protein to an electrode.

In some embodiments, the linker comprises an aromatic group.

In some embodiments, the aromatic group comprises polycyclic aromatic hydrocarbon system.

In some embodiments, by “polycyclic aromatic hydrocarbon system” it is meant to refer to a system comprising e.g., 3, 4, 5, or 6, fused benzene rings, which, in some embodiments, is in the form in a flat aromatic system.

In some embodiments, the aromatic group is selected from, without being limited thereto, pyrene, perylene, benzopyrene, oxoperylene, rubrene, perylene bisimide, styrene, anthracene, tetracene, pentacene, or any derivative thereof.

In exemplary embodiments, the aromatic group comprises pyrene or a derivative thereof.

In some embodiments, the linker further comprises an azide group.

In some embodiments, the azide group allows to bind to the PrK, for example, via an alkyne group, e.g., by “click” chemistry.

In some embodiments, the aromatic system (e.g., pyrene group) is present in one pole of the linker, and the azide group is present at the other pole of the mediator.

In some embodiments, the two groups (e.g., azide group and the aromatic group) are connected to each other by an alkyl oxide, for example, and without being limited thereto, tri-ethylene oxide, di-ethylene oxide or mono-ethylene oxide. In some embodiments, the mediator is characterized by a length of 3 to 9 or 4 to 8 Å, for example 3, 4, 5, 6, 7, 8, or 9 Å, including any value and range therebetween.

### The Porphyrin

In one embodiment, the recombinant protein (e.g., the cytochrome domain) is bound to a porphyrin comprising a metal. In one embodiment, the recombinant protein is bound to a compound of formula I:

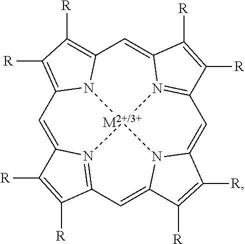

wherein R is any electron donor, or a compound as further described herein, wherein the compound of formula I is bound to a metal. In one embodiment, the recombinant protein but not the gamma subunit is bound to a porphyrin as described herein.

In one embodiment, R represents, independently and in each occurrence, a substituent selected from the group consisting of: alkyl, cycloalkyl, aryl, heteroalicyclic, heteroaryl, alkoxy, hydroxy, phosphonate, thiohydroxy, thioalkoxy, aryloxy, thioaryloxy, amino, nitro, halo, trihalomethyl, cyano, amide, amine, alkanoamine, carboxy, sulfonyl, sulfoxy, sulfinyl, and sulfonamide, or is absent. In one embodiment, R represents, independently and in each occurrence, hydrogen.

In one embodiment, R represents —(C₁-C₆)alkyl. In some embodiments, R represents —(C₁-C₆)alkoxy. In some embodiments, R represents —(C₁-C₆)alkylthio. In some embodiments, R represents —(C₁-C₆)alkylsulfinyl. In some embodiments, R represents —(C₁-C₆)alkylsulfonyl. In some embodiments, R represents —[(C₁-C₆)alkyl]NH. In some embodiments, R represents —[(C₁-C₆)alkyl]COOH.

In one embodiment, the term “alkyl” comprises an aliphatic hydrocarbon including straight chain and branched chain groups. Preferably, the alkyl group has 21 to 100 carbon atoms, and more preferably 21-50 carbon atoms. Whenever a numerical range; e.g., “21-100”, is stated herein, it implies that the group, in this case the alkyl group, may contain 21 carbon atoms, 22 carbon atoms, 23 carbon atoms, etc., up to and including 100 carbon atoms.

In one embodiment, the term “long alkyl” comprises an alkyl having at least 20 carbon atoms in its main chain (the longest path of continuous covalently attached atoms). A short alkyl therefore has 20 or less main-chain carbons. In one embodiment, an alkyl can be substituted or unsubstituted. In one embodiment, the term “alkyl”, as used herein, also encompasses saturated or unsaturated hydrocarbon, hence this term further encompasses alkenyl and alkynyl.

In one embodiment, the term “alkenyl” describes an unsaturated alkyl, as defined herein, having at least two carbon atoms and at least one carbon-carbon double bond. The alkenyl may be substituted or unsubstituted by one or more substituents, as described hereinabove. In one embodiment, the term “alkynyl”, as defined herein, is an unsaturated alkyl having at least two carbon atoms and at least one carbon-carbon triple bond. The alkynyl may be substituted or unsubstituted by one or more substituents.

In one embodiment, the term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted.

In one embodiment, the term “aryl” describes an all-carbon monocyclic or fused-ring polycyclic (i.e., rings which share adjacent pairs of carbon atoms) groups having a completely conjugated pi-electron system. In one embodiment, an aryl group may be substituted or unsubstituted.

In one embodiment, the term alkoxy” describes both an —O-alkyl and an —O-cycloalkyl group. In one embodiment, the term “aryloxy” describes an —O-aryl. In one embodiment, the term alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule.

In one embodiment, “halide”, “halogen” or “halo” describes fluorine, chlorine, bromine or iodine. In one embodiment, “haloalkyl” describes an alkyl group as defined herein, further substituted by one or more halide(s). In one embodiment, “haloalkoxy” describes an alkoxy group as defined herein, further substituted by one or more halide(s). In one embodiment, the term “hydroxyl” or “hydroxy” describes a —OH group. In one embodiment, the term “thiohydroxy” or “thiol” describes a —SH group. In one embodiment, the term “thioalkoxy” describes both an —S-alkyl group, and a —S-cycloalkyl group. In one embodiment, the term “thioaryloxy” describes both an —S-aryl and a —S-heteroaryl group. In one embodiment, the term “amine” describes a —NR′R″ group, with R′ and R″. In one embodiment, the term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

In one embodiment, the term “heteroalicyclic” or “heterocyclyl” describes a monocyclic or fused ring group having in the ring(s) one or more atoms such as nitrogen, oxygen and sulfur. The rings may also have one or more double bonds. In one embodiment, the rings do not have a completely conjugated pi-electron system. Representative examples are piperidine, piperazine, tetrahydrofurane, tetrahydropyrane, morpholino and the like.

In one embodiment, the term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon).

In one embodiment, the term “carbonyl” describes a —C(═O)—R′ group, where R′ is as defined hereinabove. In one embodiment, the above-terms also encompass thio-derivatives thereof (thiocarboxy and thiocarbonyl).

In one embodiment, the term “thiocarbonyl” describes a —C(═S)—R′ group, where R′ is as defined hereinabove. In one embodiment, the term “thiocarboxy” group describes a —C(═S)—OR′ group, where R′ is as defined herein. In one embodiment, the term sulfinyl” group describes an —S(═O)—R′ group, where R′ is as defined herein. In one embodiment, the term sulfonyl” or “sulfonate” group describes an —S(═O)2-R′ group, where R′ is as defined herein. In one embodiment, the term “carbamyl” or “carbamate” group describes an —OC(═O)—NR′R″ group, where R′ is as defined herein and R″ is as defined for R′.

In one embodiment, the term “nitro” group refers to a —NO2 group. In one embodiment, the term “cyano” or “nitrile” group refers to a —C≡T group. In one embodiment, the term azide” refers to a —N3 group. In one embodiment, the term “sulfonamide” refers to a —S(═O)2-NR′R″ group, with R′ and R″ as defined herein.

In one embodiment, the term “phosphonyl” or “phosphonate” describes an —O—P(═O)(OR′)2 group, with R′ as defined hereinabove. In one embodiment, the term “phosphinyl” describes a —PR′R″ group, with R′ and R″ as defined hereinabove.

In one embodiment, the term “alkaryl” describes an alkyl, as defined herein, which substituted by an aryl, as described herein. In one embodiment, alkaryl is benzyl.

In one embodiment, the term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

In one embodiment, the terms “halo” and “halide”, which are referred to herein interchangeably, describe an atom of a halogen, that is fluorine, chlorine, bromine or iodine, also referred to herein as fluoride, chloride, bromide and iodide. In one embodiment, the term “haloalkyl” describes an alkyl group as defined above, further substituted by one or more halide(s).

In one embodiment, the metal is a trivalent metal or a divalent metal.

In one embodiment, the recombinant protein as described herein comprises both peroxidase activity and oxidative activity. In one embodiment, the recombinant protein bound to a porphyrin comprising a metal comprises both peroxidase activity and oxidative activity. In one embodiment, the recombinant protein bound to a porphyrin comprising a metal comprises both peroxidase activity and oxidase activity.

In one embodiment, the recombinant protein is characterized by Michaelis-Menten constant K_{M}^{app} value which is at least 2, 3, 4 or 5, or more, higher compared to plain GDH, as measured under the same condition (e.g., glucose concentration) of glucose oxidation.

In one embodiment, the recombinant protein is characterized by Michaelis-Menten constant K_{M}^{app} value which is at least 2, 3, 4 or 5, or more, higher compared to plain LDH, as measured under the same condition (e.g., lactate concentration) of lactate oxidation.

In one embodiment, the recombinant protein is characterized by higher selectivity towards glucose oxidation as compared to other sugar's molecules. In one embodiment, the means that the rate of glucose oxidation is higher by at least 10%, by at least 20%, by at least 30%, by at least 40%, or by at least 50% than a other sugar molecule.

As used herein, the terms “peptide”, “polypeptide” and “protein” are used interchangeably to refer to a polymer of amino acid residues. In some embodiments, the peptides, polypeptides and proteins described herein have modifications rendering them more stable while in the body or more capable of penetrating into cells. In some embodiments, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the term “recombinant protein” refers to a protein which is coded for by a recombinant DNA and is thus not naturally occurring. The term “recombinant DNA” refers to DNA molecules formed by laboratory methods of genetic recombination. Generally, this recombinant DNA is in the form of a vector used to express the recombinant protein in a cell. In one embodiment, the recombinant protein is provided within a single composition or kit with the gamma subunit FAD-GDH. In one embodiment, the recombinant protein with the gamma subunit FAD-GDH are provided within a single composition or kit as two separate proteins (unbound).

In some embodiments, the recombinant protein is a hybrid protein. In some embodiments, the recombinant protein is a chimeric protein. In some embodiments, the c-type cytochrome peptide is from a different protein than the alpha subunit. In some embodiments, the c-type cytochrome peptide is from a different protein than the LDH. In some embodiments, the c-type cytochrome peptide is not from FAD-GDH. In some embodiments, the c-type cytochrome peptide is from a different protein than the LDH. In some embodiments, the c-type cytochrome peptide is not from FMN-LDH. In some embodiments, the c-type cytochrome peptide is not from GDH. In some embodiments, the nucleic acid sequence encoding the alpha subunit and the sequence encoding the c-type cytochrome peptide are operably linked, such that a full-length protein is produced following translation and/or transcription. In some embodiments, the nucleic acid sequence encoding the LDH and the sequence encoding the c-type cytochrome peptide are operably linked, such that a full-length protein is produced following translation and/or transcription. The term “operably linked” is intended to mean that the two nucleotide sequences of interest are linked to each other in a manner that allows for expression of the nucleotide sequence (e.g. in an in vitro transcription/translation system or in a host cell when the vector is introduced into the host cell).

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid” which refers to a circular double stranded DNA loop into which additional DNA segments can be inserted, such as by standard molecular cloning techniques. Another type of vector, wherein virally-derived DNA or RNA sequences are present in the virus (e.g. retroviruses, replication defective retroviruses, adenoviruses, replication defective adenoviruses, and adeno-associated viruses). Viral vectors also include polynucleotides carried by a virus for transfecting into host cells. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. Common expression vectors of utility in recombinant DNA techniques are often in the form of plasmids.

Recombinant expression vectors can comprise a nucleic acid coding for the protein of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

A vector or a plasmid is an artificial composite. A vector or a plasmid as described herein is man-made. A vector or a plasmid as described herein is not a product of nature.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

General methods in molecular and cellular biochemistry, such as may be useful for carrying out DNA and protein recombination, as well as other techniques described herein, can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

It should be well understood to one skilled in the art that a recombinant protein is produced by expressing the recombinant DNA in a cell and then purifying the protein. The cells expressing the DNA are cultured under effective conditions, which allow for the expression of high amounts of recombinant polypeptide. Such effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

Purification of a recombinant protein involves standard laboratory techniques for extracting a recombinant protein that is essentially free from contaminating cellular components, such as carbohydrate, lipid, or other proteinaceous impurities associated with the peptide in nature. Purification can be carried out using a tag that is part of the recombinant protein or thought immuno-purification with antibodies directed to the recombinant protein. Kits are commercially available for such purifications and will be familiar to one skilled in the art. Typically, a preparation of purified peptide contains the peptide in a highly-purified form, i.e., at least about 80% pure, at least about 90% pure, at least about 95% pure, greater than 95% pure, or greater than 99% pure.

In some embodiments, the protein comprises an amino acid sequence with at least 60%, at least 65%, at least 70%, at least 75%, at least 80%, at least 85%, at least 90%, at least 95%, at least 99% homology to the amino acid sequence set forth below in SEQ ID NO: 1. In some embodiments, the amino acid sequence with at least 70% homology to SEQ ID NO: 1 is the amino acid sequence set forth in SEQ ID NO: 6.

Mutations and deletions in a recombinant protein are created by introducing the mutation or deletion into the recombinant DNA. Methods of site-directed mutagenesis, and routine DNA recombination can be found in such standard textbooks as are enumerated above. Mutagenesis of one amino acid to another may require mutation of 1, 2, or 3 of the bases that make up the codon corresponding to the amino acid to be changed.

In some embodiments, provided herein a method for quantifying the amount of a reporter in a sample having a first detectable range of light absorbance in an oxidized state a second range of light absorbance in a non-oxidized state and, comprising: contacting the recombinant protein with the reporter in a non-oxidized state; and measuring the amount of the reporter in an oxidized state, thereby quantifying the amount of a reporter in a sample. In some embodiments, coupled is directly bound. In another embodiment, first detectable range of light absorbance is detectable in visible light and the second range of light absorbance is non-detectable in visible light. In some embodiments, the reporter is 2,6-Dichloroindophenol. In some embodiments, the reporter is coupled to glucose. In some embodiments, 2,6-Dichloroindopheno is coupled to glucose. In some embodiments, the reporter is coupled to lactate. In some embodiments, 2,6-Dichloroindopheno is coupled to lactate.

### DNA Sequences

In one embodiment, provided herein is a DNA molecule encoding the recombinant protein. In one embodiment, provided herein is a DNA molecule comprising: a transcription regulatory element, a translation regulatory element or both; operably linked to a nucleotide sequence encoding the recombinant protein. In one embodiment, the invention provides a DNA molecule encoding both the recombinant protein and the gamma subunit of an FAD-GDH. In one embodiment, the invention provides a single DNA molecule encoding both the recombinant protein and the gamma subunit of an FAD-GDH as two separate proteins.

In one embodiment, provided herein is a DNA molecule comprising a nucleic acid sequence encoding the recombinant protein. In one embodiment, provided herein is a DNA molecule comprising a nucleic acid sequence encoding the recombinant protein and the gamma subunit of an FAD-GDH. In one embodiment, provided herein is a DNA molecule comprising the nucleic acid sequence selected from the group consisting SEQ ID NOs: 5-7. In one embodiment, the invention provides a plasmid or a vector (such as an expression vector) comprising a nucleic acid sequence encoding the recombinant protein. In one embodiment, provided herein is a cell comprising a DNA molecule, a plasmid or a vector as described herein. In one embodiment, the cell is a prokaryotic cell. In one embodiment, the cell is a bacterial cell.

In one embodiment, the alpha subunit of the FAD-GDH is encoded by a DNA sequence comprising or consisting of the nucleotide sequence: ATGGCGGATACGGATACCCAGAAAGCGGACGTGGTCGTGGTTGGATCCGGCGTGGCAGG CGCAATCGTGGCTCATCAACTGGCAATGGCAGGTAAAAGCGTGATCCTGCTGGAAGCTGG TCCGCGTATGCCGCGTTGGGAAATTGTTGAACGTTTCCGCAATCAAGTCGATAAAACCGA CTTTATGGCACCGTATCCGAGCAGCGCATGGGCACCGCATCCGGAATATGGTCCGCCGAA TGATTACCTGATCCTGAAAGGCGAACACAAATTTAACTCACAGTACATTCGTGCAGTGGG CGGCACCACGTGGCATTGGGCAGCCTCGGCATGGCGCTTCATCCCGAACGATTTTAAAAT GAAAACCGTGTATGGCGTTGGTCGTGACTGGCCGATTCAGTACGATGACATCGAACATTA TTACCAACGCGCGGAAGAAGAACTGGGCGTGTGGGGTCCGGGCCCGGAAGAAGACCTGT ATTCACCGCGTAAAGAACCGTACCCGATGCCGCCGCTGCCGCTGAGTTTCAATGAACAAA CCATTAAATCCGCTCTGAACGGCTATGATCCGAAATTTCACGTGGTTACGGAACCGGTGG CCCGTAATTCGCGCCCGTACGACGGTCGCCCGACCTGCTGTGGCAACAATAACTGCATGC CGATTTGTCCGATCGGTGCAATGTATAACGGCATCGTCCATGTGGAAAAAGCTGAACAGG CAGGTGCTAAACTGATTGATAGTGCGGTCGTGTACAAACTGGAAACGGGCCCGGACAAA CGTATTACCGCAGCTGTTTATAAAGATAAAACGGGTGCGGACCATCGCGTCGAAGGCAAA TACTTCGTGATTGCGGCCAATGGTATCGAAACCCCGAAAATTCTGCTGATGAGCGCGAAC CGTGATTTTCCGAATGGTGTGGCCAACAGTTCCGATATGGTTGGCCGCAATCTGATGGAC CATCCGGGCACCGGCGTGAGCTTTTATGCAAACGAAAAACTGTGGCCGGGTCGTGGTCCG CAGGAAATGACCTCTCTGATCGGTTTCCGTGATGGCCCGTTTCGCGCGAATGAAGCAGCG AAGAAAATTCATCTGTCAAATATGTCGCGTATCAACCAGGAAACCCAAAAAATCTTTAAA GGCGGTAAACTGATGAAACCGGAAGAACTGGATGCGCAGATCCGTGACCGCAGTGCCCG CTTTGTTCAATTCGATTGCTTTCACGAAATCCTGCCGCAGCCGGAAAATCGTATTGTCCCG TCCAAAACCGCAACGGACGCAGTGGGTATTCCGCGTCCGGAAATTACGTATGCGATCGAT GACTACGTCAAACGTGGCGCAGTGCATACGCGCGAAGTTTATGCTACCGCGGCCAAAGTG CTGGGCGGCACCGAAGTGGTCTTCAACGATGAATTTGCGCCGAATAACCACATCACCGGT GCCACGATTATGGGCGCGGATGCCCGTGACTCAGTGGTTGATAAAGACTGTCGCGCCTTC GATCATCCGAACCTGTTTATTAGCAGCAGCAGCACCATGCCGACGGTTGGCACCGTTAAC GTCACCCTGACGATTGCAGCTCTGGCACTGCGTATGTCTGATACGCTGAAAAAAGAAGTC (SEQ ID NO: 1). In one embodiment, the recombinant protein is encoded by a DNA molecule comprising a coding nucleotide sequence encoding the alpha subunit of the FAD-GDH.

In one embodiment, the alpha subunit of the FAD-GDH is encoded by a DNA sequence of 1200 to 1700 nucleotides. In one embodiment, the alpha subunit of the FAD-GDH is encoded by a DNA sequence of 1200 to 1650 nucleotides. In one embodiment, the alpha subunit of the FAD-GDH is encoded by a DNA sequence of 1300 to 1650 nucleotides. In one embodiment, the alpha subunit of the FAD-GDH is encoded by a DNA sequence of 1400 to 1700 nucleotides. In one embodiment, the alpha subunit of the FAD-GDH is encoded by a DNA sequence of 1500 to 1700 nucleotides. In one embodiment, the alpha subunit of the FAD-GDH is encoded by a DNA sequence of 1500 to 1650 nucleotides. In one embodiment, the alpha subunit of the FAD-GDH is encoded by a DNA sequence of 1550 to 1640 nucleotides. In one embodiment, the alpha subunit of the FAD-GDH is encoded by a DNA sequence of 1600 to 1650 nucleotides.

In one embodiment, the alpha subunit of the FAD-GDH is a mutant of alpha FAD-GDH or a mutant of SEQ ID NO: 1. Active mutants of alpha FAD-GDH or SEQ ID NO: 1 are readily available to one of skill in the art. By the term active mutant, as used in conjunction with an FAD-GDH, is meant a mutated form of the naturally occurring FAD-GDH. FAD-GDH mutant or variants will typically but not exclusively have at least 70%, e.g., 80%, 85%, 90% to 95% or more, and for example 98% or more amino acid sequence identity to the amino acid sequence of the reference FAD-GDH molecule.

In one embodiment, the DNA sequence of a mutant of FAD-GDH or a mutant of SEQ ID NO: 1 is at least 70% identical to SEQ ID NO: 1. In one embodiment, the DNA sequence of a mutant of FAD-GDH or a mutant of SEQ ID NO: 1 is at least 75% identical to SEQ ID NO: 1. In one embodiment, the DNA sequence of a mutant of FAD-GDH or a mutant of SEQ ID NO: 1 is at least 80% identical to SEQ ID NO: 1. In one embodiment, the DNA sequence of a mutant of FAD-GDH or a mutant of SEQ ID NO: 1 is at least 85% identical to SEQ ID NO: 1. In one embodiment, the DNA sequence of a mutant of FAD-GDH or a mutant of SEQ ID NO: 1 is at least 90% identical to SEQ ID NO: 1. In one embodiment, the DNA sequence of a mutant of FAD-GDH or a mutant of SEQ ID NO: 1 is at least 95% identical to SEQ ID NO: 1. In one embodiment, the DNA sequence of a mutant of FAD-GDH or a mutant of SEQ ID NO: 1 is at least 97% identical to SEQ ID NO: 1.

In one embodiment, the gamma subunit of the FAD-GDH is encoded by a DNA sequence comprising or consisting of the nucleotide sequence:

(SEQ ID NO: 2)
ATGGCTCACAATGACAACACCCCGCACTCCCGCCGTACCGGCGATGCGGCCGTGACCGGT
ATTACGCGTCGCCAGTGGCTGCAAGGCGCGCTGGCCCTGACCGCAGCTGGCCTGACGGGT
TCCCTGGCCCTGCGCGCACTGGCTGATGATCCGGGCACCGCACCGCTGGATACCTTTATG
ACGCTGAGCGAAGCTCTGACGGGCAAAAAAGGTCTGTCTCGTGTTCTGGGCCAGCGTTTT
CTGCAAGCGCTGCAAAAAGGTTCATTCAAAACCGCGGATTCGCTGCCGCAGCTGGCGGGC
GCCCTGGCAAGCGGTTCTCTGAACCCGGACCAAGAAGCTCTGGCGCTGAAAATCCTGGAA
GCATGGTATCTGGGCATTGTTGATAATGTGGTTATCACCTACGAAGAAGCCCTGATGTTTA
GTGTCGTGTCCGACACGCTGGTCATTCCGAGCTATTGCCCGAACAAACCGGGTTTCTGGG
CCGAAAAACCGATCGAACGTCAGGCATAA.

In one embodiment, the gamma subunit of the FAD-GDH is translated into a discrete protein. In one embodiment, the gamma subunit of the FAD-GDH is encoded by a DNA sequence of 400 to 700 nucleotides. In one embodiment, the gamma subunit of the FAD-GDH is encoded by a DNA sequence of 450 to 650 nucleotides. In one embodiment, the gamma subunit of the FAD-GDH is encoded by a DNA sequence of 450 to 550 nucleotides. In one embodiment, the gamma subunit of the FAD-GDH is encoded by a DNA sequence of 480 to 530 nucleotides. In one embodiment, the gamma subunit of the FAD-GDH is encoded by a DNA sequence of 500 to 540 nucleotides. In one embodiment, the gamma subunit of the FAD-GDH is encoded by a DNA sequence of 500 to 530 nucleotides.

In one embodiment, the gamma subunit of the FAD-GDH is a mutant of gamma FAD-GDH or a mutant of SEQ ID NO: 2. In one embodiment, active mutants of gamma FAD-GDH or SEQ ID NO: 2 are readily available to one of skill in the art.

In one embodiment, the DNA sequence of a mutant of FAD-GDH or a mutant of SEQ ID NO: 2 is at least 70% identical to SEQ ID NO: 2. In one embodiment, the DNA sequence of a mutant of FAD-GDH or a mutant of SEQ ID NO: 2 is at least 75% identical to SEQ ID NO: 2. In one embodiment, the DNA sequence of a mutant of FAD-GDH or a mutant of SEQ ID NO: 2 is at least 80% identical to SEQ ID NO: 2. In one embodiment, the DNA sequence of a mutant of FAD-GDH or a mutant of SEQ ID NO: 2 is at least 85% identical to SEQ ID NO: 2. In one embodiment, the DNA sequence of a mutant of FAD-GDH or a mutant of SEQ ID NO: 2 is at least 90% identical to SEQ ID NO: 2. In one embodiment, the DNA sequence of a mutant of FAD-GDH or a mutant of SEQ ID NO: 2 is at least 95% identical to SEQ ID NO: 2. In one embodiment, the DNA sequence of a mutant of FAD-GDH or a mutant of SEQ ID NO: 2 is at least 97% identical to SEQ ID NO: 2.

In one embodiment, the minimal cytochrome peptide is encoded by a DNA sequence comprising or consisting of the nucleotide sequence:

(SEQ ID NO: 3)
ATTCGTGCAGGTGCTACCATGCCGCATCGTGATCGTGGTCCGTGCGGTGC
ATGTCACGCTATTATCCAG.

In one embodiment, the recombinant protein is encoded by a DNA sequence comprising or consisting of the nucleotide sequence:

(SEQ ID NO: 5 without linker, his and restriction sites)
CTCACAATGACAACACCCCGCACTCCCGCCGTACCGGCGATGCGGCCGTGACCGGTATTA
CGCGTCGCCAGTGGCTGCAAGGCGCGCTGGCCCTGACCGCAGCTGGCCTGACGGGTTCCC
TGGCCCTGCGCGCACTGGCTGATGATCCGGGCACCGCACCGCTGGATACCTTTATGACGC
TGAGCGAAGCTCTGACGGGCAAAAAAGGTCTGTCTCGTGTTCTGGGCCAGCGTTTTCTGC
AAGCGCTGCAAAAAGGTTCATTCAAAACCGCGGATTCGCTGCCGCAGCTGGCGGGCGCCC
TGGCAAGCGGTTCTCTGAACCCGGACCAAGAAGCTCTGGCGCTGAAAATCCTGGAAGCAT
GGTATCTGGGCATTGTTGATAATGTGGTTATCACCTACGAAGAAGCCCTGATGTTTAGTGT
CGTGTCCGACACGCTGGTCATTCCGAGCTATTGCCCGAACAAACCGGGTTTCTGGGCCGA
AAAACCGATCGAACGTCAGGCATAATGGCGGATACGGATACCCAGAAAGCGGACGTGGT
CGTGGTTGGATCCGGCGTGGCAGGCGCAATCGTGGCTCATCAACTGGCAATGGCAGGTAA
AAGCGTGATCCTGCTGGAAGCTGGTCCGCGTATGCCGCGTTGGGAAATTGTTGAACGTTT
CCGCAATCAAGTCGATAAAACCGACTTTATGGCACCGTATCCGAGCAGCGCATGGGCACC
GCATCCGGAATATGGTCCGCCGAATGATTACCTGATCCTGAAAGGCGAACACAAATTTAA
CTCACAGTACATTCGTGCAGTGGGCGGCACCACGTGGCATTGGGCAGCCTCGGCATGGCG
CTTCATCCCGAACGATTTTAAAATGAAAACCGTGTATGGCGTTGGTCGTGACTGGCCGATT
CAGTACGATGACATCGAACATTATTACCAACGCGCGGAAGAAGAACTGGGCGTGTGGGG
TCCGGGCCCGGAAGAAGACCTGTATTCACCGCGTAAAGAACCGTACCCGATGCCGCCGCT
GCCGCTGAGTTTCAATGAACAAACCATTAAATCCGCTCTGAACGGCTATGATCCGAAATT
TCACGTGGTTACGGAACCGGTGGCCCGTAATTCGCGCCCGTACGACGGTCGCCCGACCTG
CTGTGGCAACAATAACTGCATGCCGATTTGTCCGATCGGTGCAATGTATAACGGCATCGT
CCATGTGGAAAAAGCTGAACAGGCAGGTGCTAAACTGATTGATAGTGCGGTCGTGTACAA
ACTGGAAACGGGCCCGGACAAACGTATTACCGCAGCTGTTTATAAAGATAAAACGGGTG
CGGACCATCGCGTCGAAGGCAAATACTTCGTGATTGCGGCCAATGGTATCGAAACCCCGA
AAATTCTGCTGATGAGCGCGAACCGTGATTTTCCGAATGGTGTGGCCAACAGTTCCGATA
TGGTTGGCCGCAATCTGATGGACCATCCGGGCACCGGCGTGAGCTTTTATGCAAACGAAA
AACTGTGGCCGGGTCGTGGTCCGCAGGAAATGACCTCTCTGATCGGTTTCCGTGATGGCC
CGTTTCGCGCGAATGAAGCAGCGAAGAAAATTCATCTGTCAAATATGTCGCGTATCAACC
AGGAAACCCAAAAAATCTTTAAAGGCGGTAAACTGATGAAACCGGAAGAACTGGATGCG
CAGATCCGTGACCGCAGTGCCCGCTTTGTTCAATTCGATTGCTTTCACGAAATCCTGCCGC
AGCCGGAAAATCGTATTGTCCCGTCCAAAACCGCAACGGACGCAGTGGGTATTCCGCGTC
CGGAAATTACGTATGCGATCGATGACTACGTCAAACGTGGCGCAGTGCATACGCGCGAAG
TTTATGCTACCGCGGCCAAAGTGCTGGGCGGCACCGAAGTGGTCTTCAACGATGAATTTG
CGCCGAATAACCACATCACCGGTGCCACGATTATGGGCGCGGATGCCCGTGACTCAGTGG
TTGATAAAGACTGTCGCGCCTTCGATCATCCGAACCTGTTTATTAGCAGCAGCAGCACCAT
GCCGACGGTTGGCACCGTTAACGTCACCCTGACGATTGCAGCTCTGGCACTGCGTATGTCT
GATACGCTGAAAAAAGAAGTCATTCGTGCAGGTGCTACCATGCCGCATCGTGATCG
TGGTCCGTGCGGTGCATGTCACGCTATTATCCAG.

In one embodiment, the recombinant protein is encoded by a DNA sequence comprising or consisting of the nucleotide sequence:

(SEQ ID NO: 6 with linker and without his and restriction sites)
CTCACAATGACAACACCCCGCACTCCCGCCGTACCGGCGATGCGGCCGTGACCGGTATTA
CGCGTCGCCAGTGGCTGCAAGGCGCGCTGGCCCTGACCGCAGCTGGCCTGACGGGTTCCC
TGGCCCTGCGCGCACTGGCTGATGATCCGGGCACCGCACCGCTGGATACCTTTATGACGC
TGAGCGAAGCTCTGACGGGCAAAAAAGGTCTGTCTCGTGTTCTGGGCCAGCGTTTTCTGC
AAGCGCTGCAAAAAGGTTCATTCAAAACCGCGGATTCGCTGCCGCAGCTGGCGGGCGCCC
TGGCAAGCGGTTCTCTGAACCCGGACCAAGAAGCTCTGGCGCTGAAAATCCTGGAAGCAT
GGTATCTGGGCATTGTTGATAATGTGGTTATCACCTACGAAGAAGCCCTGATGTTTAGTGT
CGTGTCCGACACGCTGGTCATTCCGAGCTATTGCCCGAACAAACCGGGTTTCTGGGCCGA
AAAACCGATCGAACGTCAGGCATAATGGCGGATACGGATACCCAGAAAGCGGACGTGGT
CGTGGTTGGATCCGGCGTGGCAGGCGCAATCGTGGCTCATCAACTGGCAATGGCAGGTAA
AAGCGTGATCCTGCTGGAAGCTGGTCCGCGTATGCCGCGTTGGGAAATTGTTGAACGTTT
CCGCAATCAAGTCGATAAAACCGACTTTATGGCACCGTATCCGAGCAGCGCATGGGCACC
GCATCCGGAATATGGTCCGCCGAATGATTACCTGATCCTGAAAGGCGAACACAAATTTAA
CTCACAGTACATTCGTGCAGTGGGCGGCACCACGTGGCATTGGGCAGCCTCGGCATGGCG
CTTCATCCCGAACGATTTTAAAATGAAAACCGTGTATGGCGTTGGTCGTGACTGGCCGATT
CAGTACGATGACATCGAACATTATTACCAACGCGCGGAAGAAGAACTGGGCGTGTGGGG
TCCGGGCCCGGAAGAAGACCTGTATTCACCGCGTAAAGAACCGTACCCGATGCCGCCGCT
GCCGCTGAGTTTCAATGAACAAACCATTAAATCCGCTCTGAACGGCTATGATCCGAAATT
TCACGTGGTTACGGAACCGGTGGCCCGTAATTCGCGCCCGTACGACGGTCGCCCGACCTG
CTGTGGCAACAATAACTGCATGCCGATTTGTCCGATCGGTGCAATGTATAACGGCATCGT
CCATGTGGAAAAAGCTGAACAGGCAGGTGCTAAACTGATTGATAGTGCGGTCGTGTACAA
ACTGGAAACGGGCCCGGACAAACGTATTACCGCAGCTGTTTATAAAGATAAAACGGGTG
CGGACCATCGCGTCGAAGGCAAATACTTCGTGATTGCGGCCAATGGTATCGAAACCCCGA
AAATTCTGCTGATGAGCGCGAACCGTGATTTTCCGAATGGTGTGGCCAACAGTTCCGATA
TGGTTGGCCGCAATCTGATGGACCATCCGGGCACCGGCGTGAGCTTTTATGCAAACGAAA
AACTGTGGCCGGGTCGTGGTCCGCAGGAAATGACCTCTCTGATCGGTTTCCGTGATGGCC
CGTTTCGCGCGAATGAAGCAGCGAAGAAAATTCATCTGTCAAATATGTCGCGTATCAACC
AGGAAACCCAAAAAATCTTTAAAGGCGGTAAACTGATGAAACCGGAAGAACTGGATGCG
CAGATCCGTGACCGCAGTGCCCGCTTTGTTCAATTCGATTGCTTTCACGAAATCCTGCCGC
AGCCGGAAAATCGTATTGTCCCGTCCAAAACCGCAACGGACGCAGTGGGTATTCCGCGTC
CGGAAATTACGTATGCGATCGATGACTACGTCAAACGTGGCGCAGTGCATACGCGCGAAG
TTTATGCTACCGCGGCCAAAGTGCTGGGCGGCACCGAAGTGGTCTTCAACGATGAATTTG
CGCCGAATAACCACATCACCGGTGCCACGATTATGGGCGCGGATGCCCGTGACTCAGTGG
TTGATAAAGACTGTCGCGCCTTCGATCATCCGAACCTGTTTATTAGCAGCAGCAGCACCAT
GCCGACGGTTGGCACCGTTAACGTCACCCTGACGATTGCAGCTCTGGCACTGCGTATGTCT
GATACGCTGAAAAAAGAAGTCGAATTCGGTTCTGGTTATGGCTCTGGTCCGCCGGGTCCG
ATTCGTGCAGGTGCTACCATGCCGCATCGTGATCGTGGTCCGTGCGGTGCATGTCACGCTA
TTATCCAG.

In one embodiment, the recombinant protein is encoded by a DNA sequence comprising or consisting of the nucleotide sequence:

(SEQ ID NO: 7 with linker,
his and restriction sites)
CCATGGCTCACAATGACAACACCCCGCACTCCCGCCGTACCGGCGATGCG
GCCGTGACCGGTATTACGCGTCGCCAGTGGCTGCAAGGCGCGCTGGCCCT
GACCGCAGCTGGCCTGACGGGTTCCCTGGCCCTGCGCGCACTGGCTGATG
ATCCGGGCACCGCACCGCTGGATACCTTTATGACGCTGAGCGAAGCTCTG
ACGGGCAAAAAAGGTCTGTCTCGTGTTCTGGGCCAGCGTTTTCTGCAAGC
GCTGCAAAAAGGTTCATTCAAAACCGCGGATTCGCTGCCGCAGCTGGCGG
GCGCCCTGGCAAGCGGTTCTCTGAACCCGGACCAAGAAGCTCTGGCGCTG
AAAATCCTGGAAGCATGGTATCTGGGCATTGTTGATAATGTGGTTATCAC
CTACGAAGAAGCCCTGATGTTTAGTGTCGTGTCCGACACGCTGGTCATTC
CGAGCTATTGCCCGAACAAACCGGGTTTCTGGGCCGAAAAACCGATCGAA
CGTCAGGCATAATGGCGGATACGGATACCCAGAAAGCGGACGTGGTCGTG
GTTGGATCCGGCGTGGCAGGCGCAATCGTGGCTCATCAACTGGCAATGGC
AGGTAAAAGCGTGATCCTGCTGGAAGCTGGTCCGCGTATGCCGCGTTGGG
AAATTGTTGAACGTTTCCGCAATCAAGTCGATAAAACCGACTTTATGGCA
CCGTATCCGAGCAGCGCATGGGCACCGCATCCGGAATATGGTCCGCCGAA
TGATTACCTGATCCTGAAAGGCGAACACAAATTTAACTCACAGTACATTC
GTGCAGTGGGCGGCACCACGTGGCATTGGGCAGCCTCGGCATGGCGCTTC
ATCCCGAACGATTTTAAAATGAAAACCGTGTATGGCGTTGGTCGTGACTG
GCCGATTCAGTACGATGACATCGAACATTATTACCAACGCGCGGAAGAAG
AACTGGGCGTGTGGGGTCCGGGCCCGGAAGAAGACCTGTATTCACCGCGT
AAAGAACCGTACCCGATGCCGCCGCTGCCGCTGAGTTTCAATGAACAAAC
CATTAAATCCGCTCTGAACGGCTATGATCCGAAATTTCACGTGGTTACGG
AACCGGTGGCCCGTAATTCGCGCCCGTACGACGGTCGCCCGACCTGCTGT
GGCAACAATAACTGCATGCCGATTTGTCCGATCGGTGCAATGTATAACGG
CATCGTCCATGTGGAAAAAGCTGAACAGGCAGGTGCTAAACTGATTGATA
GTGCGGTCGTGTACAAACTGGAAACGGGCCCGGACAAACGTATTACCGCA
GCTGTTTATAAAGATAAAACGGGTGCGGACCATCGCGTCGAAGGCAAATA
CTTCGTGATTGCGGCCAATGGTATCGAAACCCCGAAAATTCTGCTGATGA
GCGCGAACCGTGATTTTCCGAATGGTGTGGCCAACAGTTCCGATATGGTT
GGCCGCAATCTGATGGACCATCCGGGCACCGGCGTGAGCTTTTATGCAAA
CGAAAAACTGTGGCCGGGTCGTGGTCCGCAGGAAATGACCTCTCTGATCG
GTTTCCGTGATGGCCCGTTTCGCGCGAATGAAGCAGCGAAGAAAATTCAT
CTGTCAAATATGTCGCGTATCAACCAGGAAACCCAAAAAATCTTTAAAGG
CGGTAAACTGATGAAACCGGAAGAACTGGATGCGCAGATCCGTGACCGCA
GTGCCCGCTTTGTTCAATTCGATTGCTTTCACGAAATCCTGCCGCAGCCG
GAAAATCGTATTGTCCCGTCCAAAACCGCAACGGACGCAGTGGGTATTCC
GCGTCCGGAAATTACGTATGCGATCGATGACTACGTCAAACGTGGCGCAG
TGCATACGCGCGAAGTTTATGCTACCGCGGCCAAAGTGCTGGGCGGCACC
GAAGTGGTCTTCAACGATGAATTTGCGCCGAATAACCACATCACCGGTGC
CACGATTATGGGCGCGGATGCCCGTGACTCAGTGGTTGATAAAGACTGTC
GCGCCTTCGATCATCCGAACCTGTTTATTAGCAGCAGCAGCACCATGCCG
ACGGTTGGCACCGTTAACGTCACCCTGACGATTGCAGCTCTGGCACTGCG
TATGTCTGATACGCTGAAAAAAGAAGTCGAATTCGGTTCTGGTTATGGCT
CTGGTCCGCCGGGTCCGATTCGTGCAGGTGCTACCATGCCGCATCGTGAT
CGTGGTCCGTGCGGTGCATGTCACGCTATTATCCAGGGCAGTGGTTCCGG
CCATCACCATCACCATCACTAAAAGCTT.

In one embodiment, the recombinant protein with or without the gamma subunit as described herein is encoded by a DNA sequence of 1500 to 3000 nucleotides. In one embodiment, the recombinant protein with or without the gamma subunit as described herein is encoded by a DNA sequence of 1600 to 2600 nucleotides. In one embodiment, the recombinant protein with or without the gamma subunit as described herein is encoded by a DNA sequence of 1800 to 2500 nucleotides. In one embodiment, the recombinant protein is encoded by a DNA sequence of 2000 to 2400 nucleotides.

In one embodiment, the recombinant protein is 350 to 700 amino acids long. In one embodiment, the recombinant protein is 220 to 600 amino acids long. In one embodiment, the recombinant protein is 250 to 550 amino acids long. In one embodiment, the recombinant protein is 450 to 850 amino acids long. In one embodiment, the recombinant protein is 470 to 750 amino acids long. In one embodiment, the recombinant protein is 500 to 700 amino acids long. In one embodiment, the recombinant protein is 710 to 780 amino acids long. In one embodiment, the recombinant protein is 500 to 600 amino acids long. In one embodiment, the recombinant protein is 520 to 580 amino acids long. In one embodiment, the recombinant protein is 350 to 550 amino acids long.

In one embodiment, a DNA sequence encoding the recombinant protein and the gamma subunit protein as described herein of SEQ ID NOs: 5 and 6 further comprises a Methionine codon (the initiation codon nucleotide sequence) 5′ to SEQ ID NOs: 5 and/or 6. In one embodiment, a DNA sequence encoding the recombinant protein of SEQ ID NOs: 5 and 6 further comprises a Methionine codon (the initiation codon nucleotide sequence) 5′ to SEQ ID NOs: 5 and/or 6. In one embodiment, a DNA sequence encoding the recombinant protein of SEQ ID NOs: 5 and 6 further comprises at its 5′ end, a short DNA sequence comprising any 1-10 nucleotides sequence. In one embodiment, a DNA sequence encoding the recombinant protein of SEQ ID NOs: 5 and 6 further comprises at its 5′ end, a short DNA sequence comprising 1-10 nucleotides sequence. In one embodiment, the 1-10 nucleotides sequence comprises the Methionine codon.

In one embodiment, a DNA sequence or molecule as described herein comprising a coding sequence encoding the recombinant protein, further encodes the gamma subunit protein as described herein (as a separate protein).

In one embodiment, the DNA sequence encoding the recombinant protein of the invention is any DNA molecule encoding the amino acid sequence encoded by anyone of SEQ ID NOs: 5-7 or the amino acid sequence of anyone of SEQ ID NOs: 8-11. In one embodiment, the DNA sequence encoding the recombinant protein with or without the gamma subunit as described herein is at least 70% identical to anyone of SEQ ID NOs: 5-7. In one embodiment, the DNA sequence encoding the recombinant protein with or without the gamma subunit as described herein is at least 75% identical to anyone of SEQ ID NOs: 5-7. In one embodiment, the DNA sequence encoding the recombinant protein with or without the gamma subunit as described herein is at least 80% identical to anyone of SEQ ID NOs: 5-7. In one embodiment, the DNA sequence encoding the recombinant protein with or without the gamma subunit as described herein is at least 85% identical to anyone of SEQ ID NOs: 5-7. In one embodiment, the DNA sequence encoding the recombinant protein with or without the gamma subunit as described herein is at least 90% identical to anyone of SEQ ID NOs: 5-7. In one embodiment, the DNA sequence encoding the recombinant protein with or without the gamma subunit as described herein is at least 95% identical to anyone of SEQ ID NOs: 5-7. In one embodiment, the DNA sequence encoding the recombinant protein with or without the gamma subunit as described herein is at least 95% identical to anyone of SEQ ID NOs: 5-7.

In some embodiments, a DNA molecule of the invention or a DNA sequence described herein comprises or consists any sequence encoding the recombinant protein including (but not limited to) the recombinant protein comprising or consisting anyone of SEQ ID NOs: 8-11. In some embodiments, a DNA molecule of the invention or a DNA sequence described herein comprises or consists any sequence encoding the recombinant protein and the gamma subunit, as described herein, including (but not limited to) the amino acid sequences set forth in anyone of SEQ ID NOs: 8-11. In one embodiment, the recombinant protein and/or the gamma subunit, as described herein is/are translated based on the DNA sequences provided herein. In one embodiment, the recombinant protein has an amino acid sequence that is at least 70%, 80%, 90%, 95%, or 97% identical to: (a) a recombinant protein translated from the DNA sequences provided herein or (b) anyone of SEQ ID NOs: 8-11.

In some embodiments, the protein of the invention comprises a tag. In some embodiments, the DNA encoding the proteins of the invention comprises sequence encoding the tag. In some embodiments, the tag is selected from an n-terminal tag, a c-terminal tag and an internal tag. A skilled artisan will appreciate that the tag should be positioned so as not to interfere with the function of the recombinant protein. Thus, the tag will not interfere with the redox activity or the DET activity. In some embodiments, the tag is a c-terminal tag. In some embodiments, the tag is a His tag. In some embodiments, the tag is a 6×His tag. In some embodiments, the His tag comprises or consists of the amino acid sequence HHHHHH. In some embodiments, the DNA encoding the His tag comprises the sequence CATCACCATCACCATCAC (SEQ ID NO: 24) (e.g., in addition to SEQ ID NO: 19-23). A skilled artisan will appreciate that any sequence which encodes the tag may be used. Protein tags are well known in the art and include, but are not limited to, HA tags, His tags, GFP tags, Myc tags, biotin tags, FLAG tags, streptavidin tags, and many, many others. Tagging may be useful for purification of the protein, and the tag may be cleaved before the enzyme is used. In some embodiments, the tag is small. In some embodiments, the tag is equal to or smaller than 40, 35, 30, 25, 20, 15, 10, 7, or 5 amino acids. Each possibility represents a separate embodiment of the invention. A smaller tag may be advantageous in that it is less likely to interfere with DET.

In some embodiments, the tag is connected to the recombinant protein by a linker. The linker may be a linker such as has been described hereinabove. In some embodiments, the linker comprises or consists of the sequence GSGSG. In some embodiments, the DNA sequence that encodes the linker comprises or consists of the sequence GGCAGTGGTTCCGGC (SEQ ID NO: 25) (e.g., in addition to SEQ ID NO: 19-23). A skilled artisan will appreciate that any sequence which encodes the linker may be used. In some embodiments, the linker is produced by the restriction site introduced into the DNA to produce the recombinant protein. Indeed, there may be a linker produced by restriction site insertion between any of the different parts of the recombinant protein.

### General

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, and material arts.

As used herein, the term “treating” includes abrogating, substantially inhibiting, slowing or reversing the progression of a condition, of aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples which, together with the above descriptions, illustrate the invention in a non-limiting fashion.

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8^{th} Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Plasmid Construction

FAD-GDH γ subunit as well as its catalytic a subunit were cloned into pTrcHis6A2 vector between NcoI and HindIII restriction sites. The partial FAD-GDH gene was followed by a short flexible polypeptide linker (13 amino-acids long) and the MCR2 (minimal cytochrome domain—MCD) DNA sequence with 6×His tag at the sequence's C-terminal, as shown in FIG. 6 in a map of the new fusion protein. For the WT GDH the same construct was used, but without the MCD sequence. Full DNA construct: CCATGGCTCACAATGACAACACCCCGCACTCCCGCCGTACCGGCGATGCGGCCGTGACCG GTATTACGCGTCGCCAGTGGCTGCAAGGCGCGCTGGCCCTGACCGCAGCTGGCCTGACGG GTTCCCTGGCCCTGCGCGCACTGGCTGATGATCCGGGCACCGCACCGCTGGATACCTTTAT GACGCTGAGCGAAGCTCTGACGGGCAAAAAAGGTCTGTCTCGTGTTCTGGGCCAGCGTTT TCTGCAAGCGCTGCAAAAAGGTTCATTCAAAACCGCGGATTCGCTGCCGCAGCTGGCGGG CGCCCTGGCAAGCGGTTCTCTGAACCCGGACCAAGAAGCTCTGGCGCTGAAAATCCTGGA AGCATGGTATCTGGGCATTGTTGATAATGTGGTTATCACCTACGAAGAAGCCCTGATGTTT AGTGTCGTGTCCGACACGCTGGTCATTCCGAGCTATTGCCCGAACAAACCGGGTTTCTGG GCCGAAAAACCGATCGAACGTCAGGCATAATGGCGGATACGGATACCCAGAAAGCGGAC GTGGTCGTGGTTGGATCCGGCGTGGCAGGCGCAATCGTGGCTCATCAACTGGCAATGGCA GGTAAAAGCGTGATCCTGCTGGAAGCTGGTCCGCGTATGCCGCGTTGGGAAATTGTTGAA CGTTTCCGCAATCAAGTCGATAAAACCGACTTTATGGCACCGTATCCGAGCAGCGCATGG GCACCGCATCCGGAATATGGTCCGCCGAATGATTACCTGATCCTGAAAGGCGAACACAAA TTTAACTCACAGTACATTCGTGCAGTGGGCGGCACCACGTGGCATTGGGCAGCCTCGGCA TGGCGCTTCATCCCGAACGATTTTAAAATGAAAACCGTGTATGGCGTTGGTCGTGACTGG CCGATTCAGTACGATGACATCGAACATTATTACCAACGCGCGGAAGAAGAACTGGGCGTG TGGGGTCCGGGCCCGGAAGAAGACCTGTATTCACCGCGTAAAGAACCGTACCCGATGCCG CCGCTGCCGCTGAGTTTCAATGAACAAACCATTAAATCCGCTCTGAACGGCTATGATCCG AAATTTCACGTGGTTACGGAACCGGTGGCCCGTAATTCGCGCCCGTACGACGGTCGCCCG ACCTGCTGTGGCAACAATAACTGCATGCCGATTTGTCCGATCGGTGCAATGTATAACGGC ATCGTCCATGTGGAAAAAGCTGAACAGGCAGGTGCTAAACTGATTGATAGTGCGGTCGTG TACAAACTGGAAACGGGCCCGGACAAACGTATTACCGCAGCTGTTTATAAAGATAAAAC GGGTGCGGACCATCGCGTCGAAGGCAAATACTTCGTGATTGCGGCCAATGGTATCGAAAC CCCGAAAATTCTGCTGATGAGCGCGAACCGTGATTTTCCGAATGGTGTGGCCAACAGTTC CGATATGGTTGGCCGCAATCTGATGGACCATCCGGGCACCGGCGTGAGCTTTTATGCAAA CGAAAAACTGTGGCCGGGTCGTGGTCCGCAGGAAATGACCTCTCTGATCGGTTTCCGTGA TGGCCCGTTTCGCGCGAATGAAGCAGCGAAGAAAATTCATCTGTCAAATATGTCGCGTAT CAACCAGGAAACCCAAAAAATCTTTAAAGGCGGTAAACTGATGAAACCGGAAGAACTGG ATGCGCAGATCCGTGACCGCAGTGCCCGCTTTGTTCAATTCGATTGCTTTCACGAAATCCT GCCGCAGCCGGAAAATCGTATTGTCCCGTCCAAAACCGCAACGGACGCAGTGGGTATTCC GCGTCCGGAAATTACGTATGCGATCGATGACTACGTCAAACGTGGCGCAGTGCATACGCG CGAAGTTTATGCTACCGCGGCCAAAGTGCTGGGCGGCACCGAAGTGGTCTTCAACGATGA ATTTGCGCCGAATAACCACATCACCGGTGCCACGATTATGGGCGCGGATGCCCGTGACTC AGTGGTTGATAAAGACTGTCGCGCCTTCGATCATCCGAACCTGTTTATTAGCAGCAGCAG CACCATGCCGACGGTTGGCACCGTTAACGTCACCCTGACGATTGCAGCTCTGGCACTGCG TATGTCTGATACGCTGAAAAAAGAAGTCGAATTCGGTTCTGGTTATGGCTCTGGTCCGCC GGGTCCGATTCGTGCAGGTGCTACCATGCCGCATCGTGATCGTGGTCCGTGCGGTGCATG TCACGCTATTATCCAGGGCAGTGGTTCCGGCCATCACCATCACCATCACTAAAAGCTT (SEQ ID NO: 26). The first 6 nucleotides and the last 6 nucleotides are NcoI and HindIII restriction sites. The Gamma subunit is expressed as a separate protein encoded from nucleotide 3 to nucleotide 512 of the DNA molecule described herein. The Alpha subunit is from nucleotide 512 to nucleotide 2128. The flexible linker is from nucleotide 2129 to nucleotide 2167. The carboxy terminal MCD linker is from nucleotide 2168 to nucleotide 2238. 6 times His-Tag from nucleotide 2239 to nucleotide 2269.

### Enzyme Expression and Purification

The complete pTrcHis6A2-FAD-GDH-MCD plasmid was transformed into E-coli BL21 cells for the expression of the fusion protein (FAD-GDH-MCD). Bacteria were cultured in an auto-induction medium (Formedium™, Hunstanton, England) with 0.5% glycerol (Bio-Lab ltd., Jerusalem, Israel) and 10 μg/mL carbenicillin (Apollo, Manchester, England) and grown in 37° C., with shaking at 250 rpm, for 6 hours after which cells were transferred to 27° C. for 18 additional hours.

The cells were then centrifuged at 6,000 rpm for 10 minutes, the pallet was resuspended using 20 mL 50 mM Tris-base buffer (TB, Fisher scientific, Geel, Belgium) pH=7.0, centrifuged, weighted, and resuspended using lysis buffer (300 mM KCl, 50 mM KH2PO4, 10 mM imidazole pH=7.0) in a 1:3 (weight:buffer volume) ratio. Protease inhibitors, lysozyme and super nuclease were added to the suspension in 1:500, 1:400, and 1:5000 ratios, respectively. The cells were lysed using sonication needle followed by 15 minutes of 50° C. incubation and centrifugation (11,500 rpm for 25 minutes). The supernatant was filtered using 0.22 μm filter.

The fusion enzyme was purified using IMAC purification system (Bio-Rad, Profinia, Hercules, CA, USA) according to manufacturer instructions.

### FAD-GDH Activity Assay

To assess FGM's D-glucose oxidation activity, glucose oxidation was measured in the presence of 0.6 mM 2,6-Dichloroindophenol (DCIP, Sigma-Aldrich, Rehovot, Israel), 0.6 mM phenazine methyl sulfate (PMS, Tokyo chemical industry, Tokyo, Japan), different concentrations of D-glucose and 0.2 mM FGM, all dissolved in 50 mM TB pH=7.0. Assay was performed in 37° C. using a 96-well plate reader (BioTek instruments, Winoosky, VT, USA) with shaking between measurements, monitoring the decrease in the DCIP absorbance=610 nm every 15 seconds over 25 min of activity.

### Heme Activity Measurements

To verify the attachment of a porphyrin to the MCD domain, heme activity was measured using 1 mM dimethoxybenzidine (DMB, Alfa Aesar, Heysham, England), 1 mM hydrogen peroxide (Sigma-Aldrich, Rehovot, Israel), and 0.2 mM of the enzyme. Measurements were performed at 37° C. using a 96-wells plate reader, monitoring the increase in DMB absorbance=455 nm every 15 seconds over 30 min of activity.

### Peroxidase Activity Interference Measurements

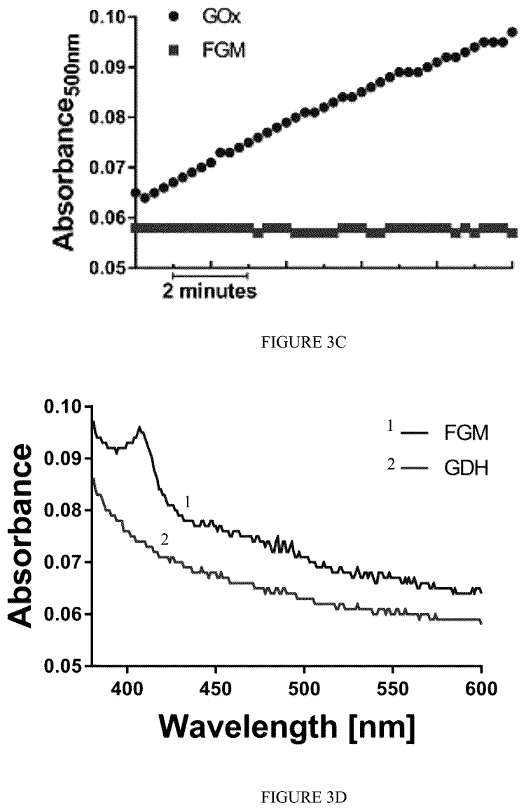

To verify that FGM does not transfer electrons to oxygen, producing hydrogen peroxide, that interferes with the FAD-GDH activity assay, peroxidase activity test was performed in the absence of hydrogen peroxide. 0.17 mM DMB and 172 mM glucose were mixed with 8 μL of 0.1 mg/mL Horseradish peroxidase (HRP, Sigma-Aldrich, Rehovot, Israel) to generate a reaction mix. 8 μL of concentrated FGM or glucose oxidase (GOx) were added to the reaction mix right before measurement. Measurements were performed at 37° C. using a plate reader, monitoring the increase in DMB absorbance=500 nm every 15 seconds over 25 min of activity (FIG. 3C).

### Electrode Preparation

Glassy carbon electrodes (GCE, 3 mm in diameter; ALS, Tokyo, Japan) were polished using 0.05 μm alumina slurry on polishing pad for two minutes, then transferred to a 20 mL glass with 10 mL double-distilled water (DDW) and sonicated for five minutes in a sonication bath. The electrodes were then dried under nitrogen gas. 10 μL of enzyme solution in wanted concentrations was dropped on the electrode surface. The electrodes were incubated in 4° C. overnight to generate an enzyme film on the GCE. Electrode's surfaces were then covered with 12-14 kDa dialysis membrane (Membrane Filtration Products, Seguin, TX, USA) and tightened using an O-ring rubber to prevent diffusion of the enzyme to the solution.

### Electrochemical Measurements

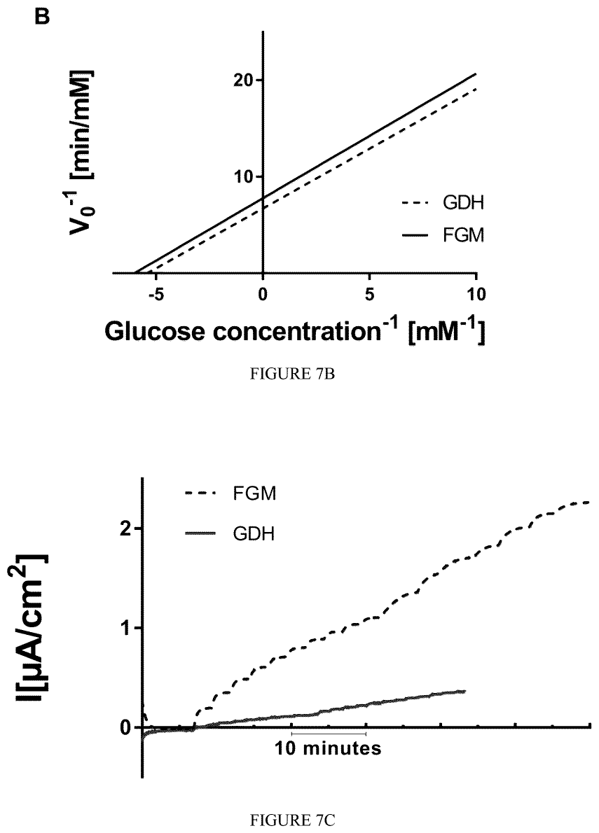

Cyclic voltametric measurements were performed using a PalmSense2 potentiostat (Palm Instruments, Houten, The Netherlands) using a standard three electrodes system with 0.9 mm graphite rod as an auxiliary electrode, 3 M KCl saturated Ag/AgCl reference electrode (ALS, Tokyo, Japan) and GCE as the working electrode in 0.15 M phosphate-citrate buffer pH=5.0. Chronoamperometric measurements (FIG. 7) were performed under the same conditions with the application of 0 mV vs. Ag/AgCl with the addition of varying concentrations of glucose or potential interfering molecules. Square wave voltammetry (SWV) measurements were performed under the same conditions.

### FGM Selectivity Test

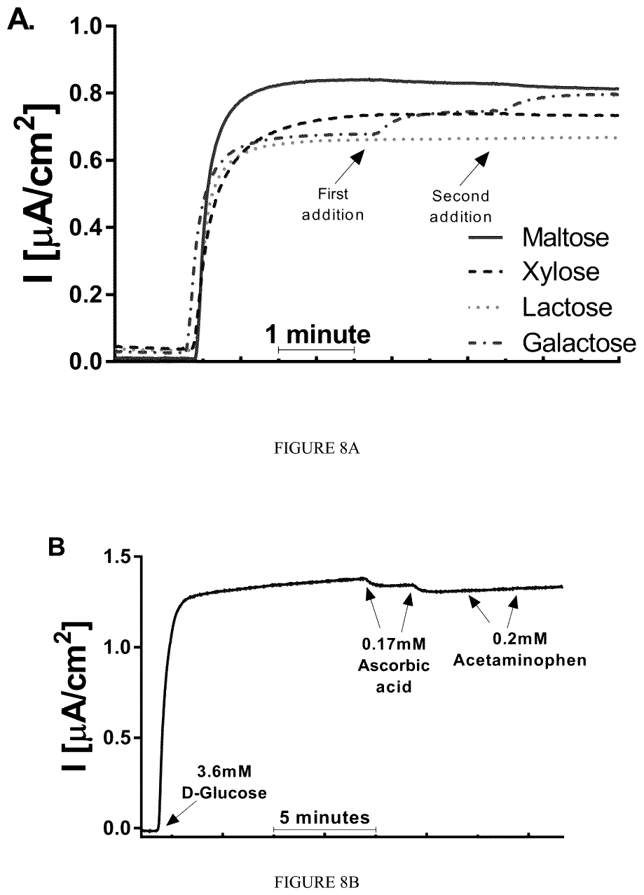

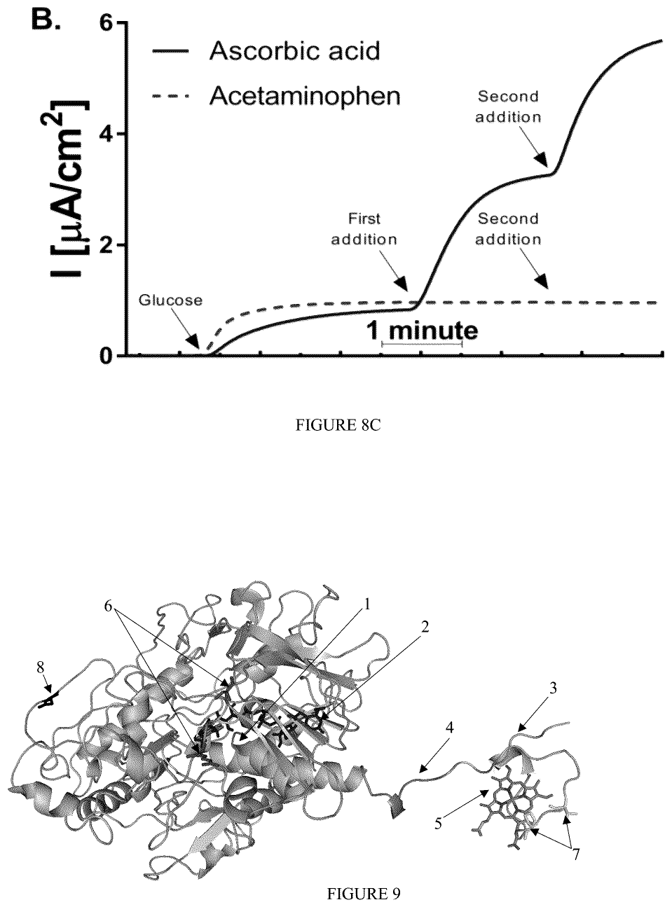

The interference of different sugars and molecules on glucose biosensing by GCE/FGM was measured using chronoamperometry (see selectivity FIGS. 8A-8C). Measurements were performed in 0.15 M phosphate-citrate buffer pH=5.0 with the application of 0 mV vs. Ag/AgCl reference electrode. FGM selectivity was tested by adding 3.6 mM of glucose followed by two sequential additions of one of the different sugars or molecules in their relevant physiological concentrations. The sugars used for this test were D-galactose, lactose, D-maltose and D-xylose and the molecules were ascorbic acid and acetaminophen (all from Sigma-Aldrich, Rehovot, Israel). Selectivity test have revealed a small interference caused by galactose while other sugars did not interfere with the measurement. This result indicates high selectivity towards glucose and low/no reaction with other sugars that can be found in human blood samples, which is very important for biosensing accuracy. GCE\FGM showed low sensitivity to ascorbic acid and no sensitivity to acetaminophen.

### The Biocatalytic Recombinant Protein

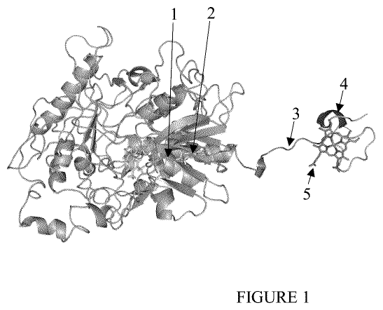

In the present study, a fusion enzyme was designed in a combination of a biocatalytic function from a redox enzyme domain that was fused to a natural minimal ET domain via a short polypeptide linker as shown in FIG. 1. As the catalytic domain, the a subunit of an FAD-GDH from Burkholderia cepacia was used. As a minimal ET unit, the c-type cytochrome domain MCR-2 from a MamP protein which originates from a magnetotactic bacteria magnetoovoid bacterium MO-127 was chosen. MamP is part of the magnetosome, a unique organelle that is found in magnetotactic bacteria that allows magneto taxis to occur in these bacteria. MCR-2 is one of the shortest natural c-type cytochromes known today (23 amino-acids long), thus can be used to achieve DET.

In order for FAD-GDH-MCD (FGM) fused enzyme to mature properly in the host cell, FAD-GDH α subunit should correctly fold. The Enzyme's γ subunit aids in the maturation of the α subunit and locates it in the periplasm. Within the bacterial periplasmatic environment, the maturation of c-type cytochromes (heme binding cytochromes) occurs with the help of a specific gene cluster called ccmA-H29. In that manner, the holo-enzyme is being transferred to the periplasm and there the MCD's maturation process occurs.

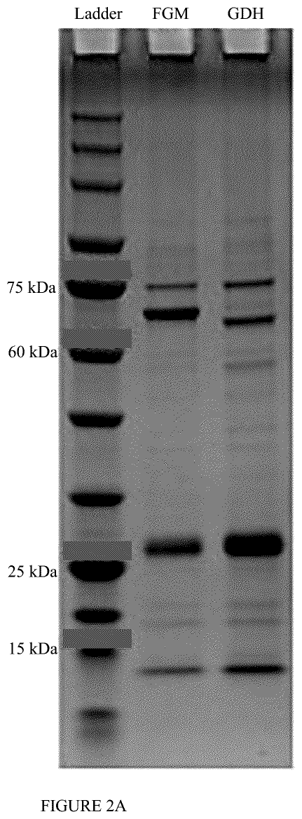

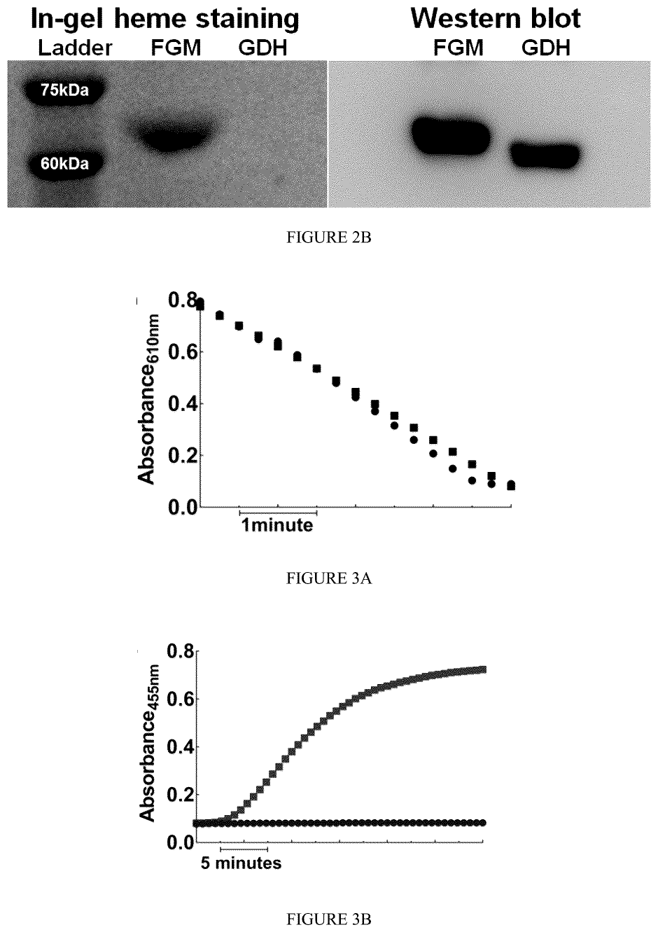

Fusion enzyme's engineered DNA sequence was cloned into pTrcHis6A2 expression vector and was transformed into E-coli BL21. FGM was overexpressed in the bacterial expression system and then purified by utilizing immobilized metal affinity chromatography (IMAC) purification system (FIG. 2A). In-gel heme staining was performed to verify the presence of the heme compared to GDH and Anti his-tag Western blot analysis was performed in order to verify the full-length enzyme's expression (FIG. 2B, right panel). As shown in FIG. 2B, both FGM and GDH enzymes were expressed and their respective bands appeared in the expected size—ca. 64 kDa and 62 kDa for FGM and GDH, respectively. In-gel heme staining revealed a band for FGM only, indicating the presence of a porphyrin containing iron bound to FGM.

FGM catalytic redox activity and heme peroxidase activity were measured biochemically and compared to GDH as shown in FIG. 3A. FGM has oxidized D-glucose as was measured by FAD-GDH activity assay in 50 mM Tris-base (pH 7.0), 0.6 mM 2,6-Dichloroindophenol (DCIP) and 0.6 mM phenazine methyl sulfate (PMS) in 37° C. Absorbance (lambda=610 nm) was monitored using a plate reader while the oxidized DCIP (blue color) was being reduced by FGM to its reduced form (colorless). FGM has also shown peroxidase activity, measured by heme activity assay in 1 mM 3,3′-dimethoxybenzidine (DMB) and 1 mM of hydrogen peroxide. Absorbance in 455 nm was monitored while the DMB oxidation occurs by the MCD, resulted in an oxidized DMB (red color). Heme activity assay results indicate that FGM indeed binds a heme group while no heme molecules are bound by GDH.

Absorbance measurements of protein sample spectrum revealed a peak in absorbance at 408 nm for FGM and no peak for GDH, indicating presence of heme c in FGM (FIG. 3B). 408 nm/A280 nm ratio was calculated to be 0.4 for FGM expressed in the presence of pEC86 plasmid, compared to 0.2 for FGM expressed in the absence of this plasmid, indicating more efficient heme maturation in the presence of the helper plasmid.

The apparent kinetic and thermodynamic parameters of FGM were calculated using FAD-GDH biochemical activity assay in 37° C. (Table 1). FGM and GDH concentrations were first determined spectrophotometrically using a standard Bradford assay. Michaelis-Menten curves were transformed to Lineweaver-Burk curves in order to determine the kinetic and thermodynamic parameters of the enzyme. One enzyme activity unit was defined as amount of enzyme oxidizing 1 μM of substrate per minute. The molar absorption coefficient of DCIP was calculated to be 4.7 cm⁻¹ mM⁻¹. By using the biochemical activity assay and DCIP molar absorption coefficient, FGM and GDH specific activity were calculated to be 16 mU.

Lineweaver-Burk plots (FIGS. 7A-7B) were used to calculate kinetic and thermodynamic parameters of the enzyme. K_{M}^{app} values were 157±5 μM and 174±9 μM for FGM and GDH, respectively, showing different affinity of the enzymes toward the substrate. GDH's K_{M}^{app} value is lower than reported values but yet in the same order of magnitude of some. K_{M}^{app} value was ca. 3 times higher for FGM compared to GDH, indicating faster oxidation of D-glucose by FGM. FGM also showed more than 3 times higher catalytic efficiency (k) compared to GDH (Table 1). Next, the electrochemical activity of the enzymes was measured to determine whether the addition of the minimal cytochrome domain improves enzyme-electrode ET.

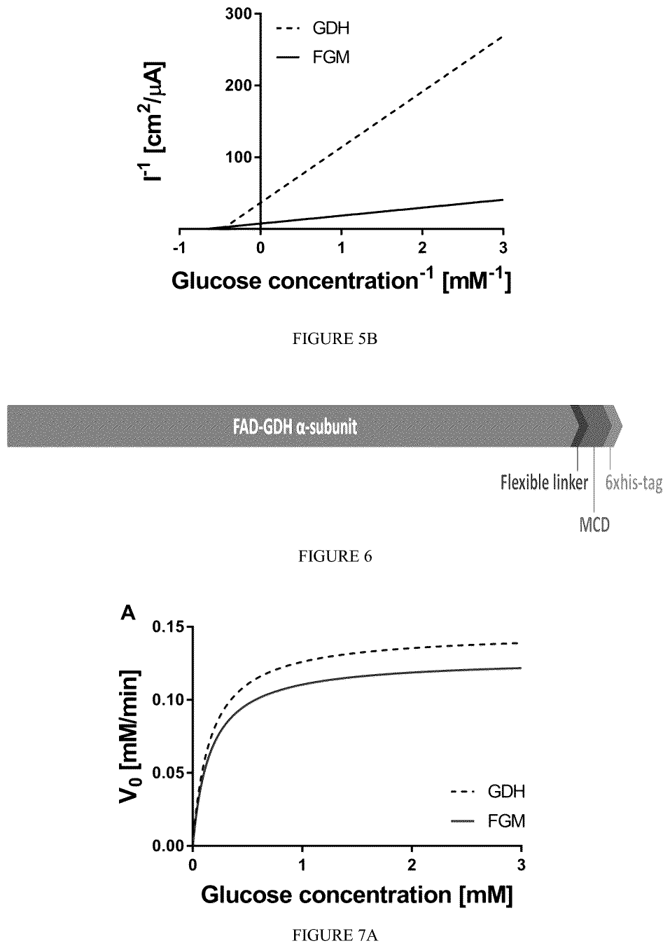

Chronoamperometric measurements (FIG. 7C) were performed to determine the apparent kinetic parameters of FGM compared to GDH. Using a standard 3 electrode electrochemical cell, the current was measured vs. successive glucose additions. A potential of 0V was applied during the measurements. The current for each glucose concentration was determined and is presented in FIG. 5A. As described above, using the linear part of the transient curves and Linewaver-Burk transformation—the electrochemical kinetic constants were calculated (FIG. 5B).

TABLE 1
Apparent biochemical kinetic
parameters of FGM and GDH

| | k_{cat}^{app} | K_{M}^{app} | k_{cat}^{app}/K_{M}^{app} |
|---|---|---|---|
| | (s⁻¹) | (μM) | (s⁻¹ · mM⁻¹) |
| GDH | 1.7 ± 0.1 | 174 ± 9 | 9.6 ± 0 |
| FGM | 5.2 ± 0.1 | 157 ± 5 | 33 ± 0 |

For the electrochemical measurements, a standard 3 electrode electrochemical cell with 0.9 mm graphite rod as the auxiliary electrode were used, 3M KCl saturated Ag/AgCl reference electrode and 3 mm diameter glassy carbon electrode (GCE) as the working electrode. 10 μL of ca. 30 μg/mL FGM or GDH enzyme solution were dropped on the GCE surface and dried in 4° C. overnight. The electrodes surface was then covered with 12-14 kDa dialysis membrane tightened to the surface with an O-ring to keep the enzyme close to the electrode surface during measurements and to avoid enzyme diffusion to the surrounding buffer. Cyclic voltammetry (CV) measurements were performed to compare the enzyme-electrode communication of FGM to that of GDH.

Optimal pH value for electrochemical measurements was tested by measuring the catalytic current in pH values of 3.6-7.0 and found to be 5.0.

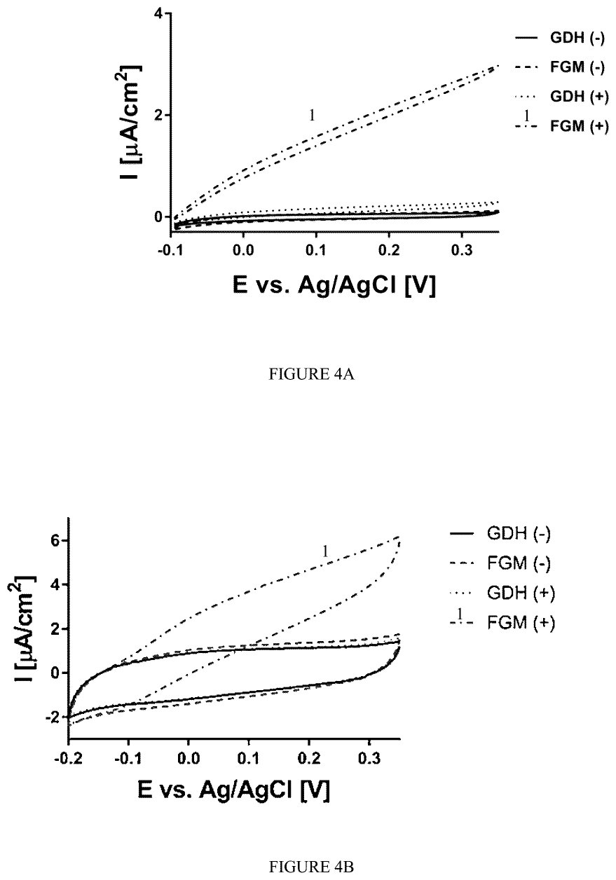

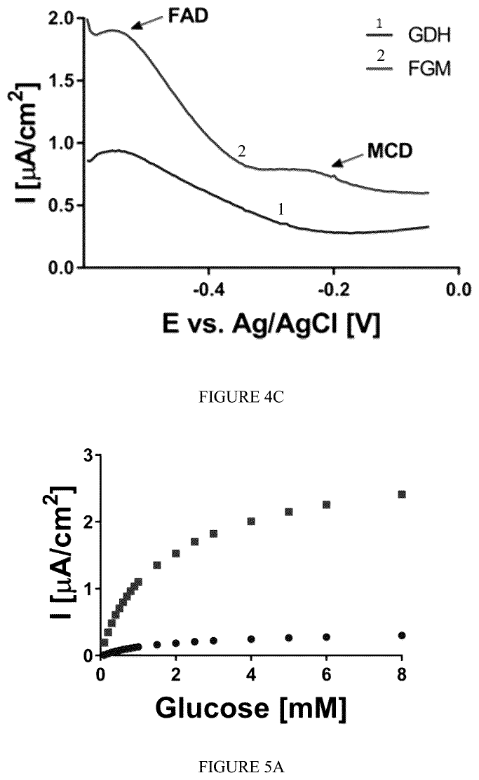

Measurements were performed in phosphate-citrate buffer pH=5.0 at room temperature and a scan rate of 5 mV/sec for both enzymes with and without the addition of 5 mM glucose. It can be seen in FIGS. 4A-B that the CVs of both enzymes before the addition of glucose are almost identical. No clear anodic or cathodic peaks were identified for both enzymes. After the addition of glucose to a final concentration of 5 mM, FGM has demonstrated 10 times higher electrocatalytic current compared to that of GDH with an onset potential of ca. −100 mV for both enzymes (indicating no apparent shift in potentials due to the fusion of MCD). That difference in the ET efficiency is probably due to the addition of the minimal cytochrome domain that mediates the ET from the buried FAD co-factor to GCE.

After the addition of glucose to a final concentration of 5 mM, FGM demonstrated a higher electrocatalytic current compared to that of GDH with an onset potential of ca. (−) 150 mV. The fact that no catalytic current was observed at this high scan rate using GDH, but a significant catalytic current evolved using FGM, is an indication of fast ET rates of FGM. That observed ET efficiency is probably due to addition of the minimal cytochrome domain that mediates ET from the buried FAD cofactor to GCE. To identify a peak originating from the MCD domain, square-wave voltammetry (SWV) was performed. As shown in FIGS. 4C, the voltammogram of an electrode with FGM revealed a peak around (−) 230 mV, whereas GDH had no observable peak at this potential.

As shown in Table 2, the electrochemical K_{M}^{app} is 2.84±0.57 mM for GDH and 1.40±0.27 mM for FGM, indicating no significant difference in the affinity towards glucose. The iₘₐₓ value is one order of magnitude higher for FGM compared to GDH−2.04±0.45 μA·cm⁻² and 0.4±0.17 μA·cm⁻², respectively. The difference in the iₘₐₓ value is indicative that the DET efficiency is different between the two enzymes where FGM shows five to seven times higher current than GDH for the same glucose concentrations.

TABLE 2
Apparent electrochemical
kinetic and thermodynamic
parameters of FGM and GDH

| | K_{M}^{app} | iₘₐₓ |
|---|---|---|
| | (mM) | (μA · cm⁻²) |
| GDH | 2.8 ± 0.6 | 0.4 ± 0.2 |
| FGM | 1.4 ± 0.3 | 2.0 ± 0.5 |

Burkholderia Cepacia is considered a good candidate for biosensing applications because of its stability in high temperatures and insensitivity to oxygen. By adding a minimal cytochrome domain to FAD-GDH c-terminus an improved DET was provided, showing higher catalytic currents compared to GDH with almost the same affinity to the substrate. When tested electrochemically with 0 V induced potential vs Ag/AgCl, FGM showed much higher currents for the same substrate concentrations compared to GDH, which makes it more accurate for glucose biosensing with improved sensitivity than previously reported for GDH.

Non-Canonical Amino Acids (ncAAs) Incorporation into FGM for Site-Specific Wiring to an Electrode: FGM Electron Transfer Machineries Investigation

To incorporate non-canonical amino acids (ncAAs) into FGM, a few constructs containing the amber (TAG) mutation on pTrcHis6A2-FGM plasmid were prepared using standard site-directed mutagenesis PCR protocol. The mutations were chosen with a proximity to the protein different domains—FAD binding domain, MCD and one site that is distant from either FAD domain or MCD. For proximity to FAD binding domain, two sites were found to be possible for ncAA incorporation—R42X and S247X (FIG. 9—red), both ca. 11 Å from FAD binding domain. For proximity to MCD, T558X and P560X (yellow) are possible sites for incorporation, with 10 and 7 Å from MCD, respectively. D395X (black) was found to be far from both FAD and MCD as it is about 37 Å from FAD and 83 Å from MCD.

Mutated plasmids were transformed into super competent E. coli DH5a cells and were plated on selective LB-agar plates. Bacterial colonies were isolated and plasmids were purified using miniprep kit followed by sequencing.

Non-Canonical Amino Acids (ncAA) Incorporation into FGM

Plasmids, with amber codon-containing FGM mutants sequences were co-transformed to E. coli BL21 strain containing the pEVOL plasmid expressing Pyrrolysyl orthogonal translation system to incorporate Propargyl-lysine (PrK) into the protein sequence. PrK containing protein was expressed in 20 mL auto-induction medium (AIM) in the presence of 1 mM PrK, lysed using Bugbuster lysis solution and was isolated utilizing IMAC purification method. PrK is an example to a clickable ncAA, all clickable biorthogonal chemical handles can be considered for the site-specific wiring of this enzyme.

### Pyrene-Azide Linkers for Enzyme Wiring to Electrode

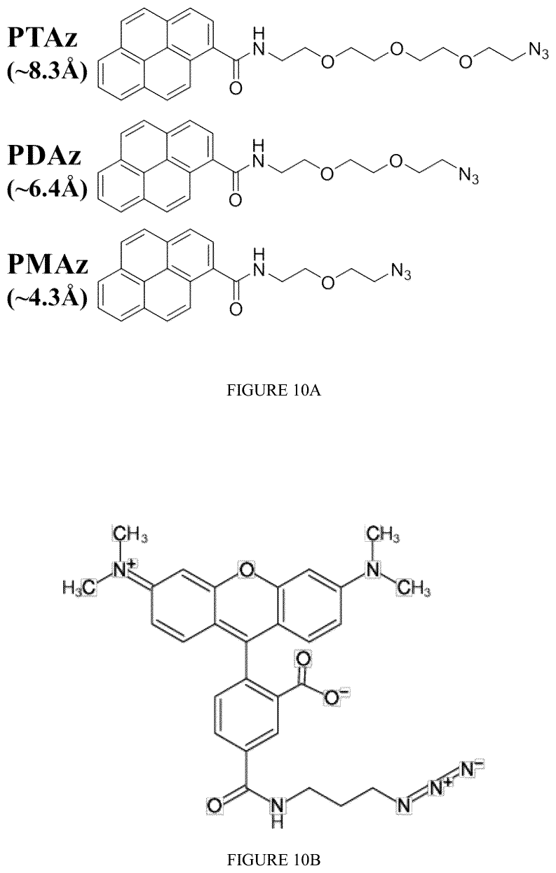

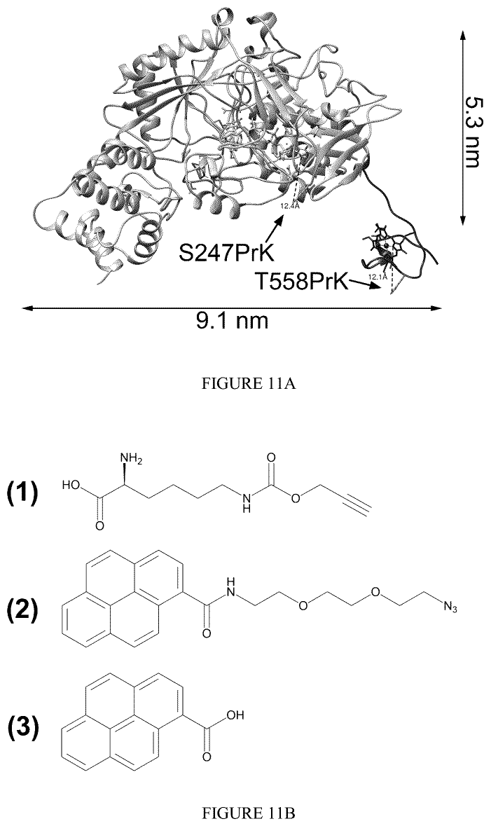

In order to wire FGM with site-specifically incorporated PrK to an electrode, a synthetic linker was used. The synthetic linker contained a pyrene group in one pole and an azide group at the other. The two groups were connected by a tri-ethylene oxide, di-ethylene oxide or mono-ethylene oxide, to get three different lengths of 8.3, 6.4 and 4.3 Å, respectively. The pyrene group is a polycyclic aromatic hydrocarbon consisting of four fused benzene rings, results in a flat aromatic system. Due to overlapping of n-bonds between aromatic side chains, the pyrene group can be attached to glassy carbon electrodes surface through π-π stacking. The azide group was used to attach the alkyne group of PrK using “click” chemistry. Exemplary pyrene-azide linker structures with different lengths are presented in FIGS. 10A-10B.

### Click Reaction:

Copper-catalyzed azide-alkyne cycloaddition (CuAAC) reaction is based on the formation of 1,4-disubstituted 1,2,3-triazoles between a terminal alkyne and an aliphatic azide in the presence of copper. The reaction is a facile, selective, high yielding with mild conditions and with few or no byproducts. It can be performed in room-temperature, what makes it relevant for use in proteins. CuAAC was used to link FGM to a pyrene-azide synthetic linker. Click reaction mix contained tris-KCl buffer (pH=8.2), pyrene-azide linker, Cu⁺, Tris(3-hydroxypropyltriazolylmethyl)amine (THPTA), FGM with incorporated PrK and sodium-ascorbate (Na-Asc). Reaction mix was incubated in RT for 30-60 min with moderate mixing, centrifuged for 10 min, and the supernatant was collected for further examinations.

ncAA Incorporation Validation

TAMRA-azide is an azide-linked reporter tag that can be used for visualization of alkyne containing proteins. To validate incorporation of PrK into FGM sequence, purified FGM was clicked with the florescent marker TAMRA-azide. Clicked protein sample was loaded on SDS-PAGE and the protein gel was checked for florescence using LAS4000 camera. In addition, anti 6×His-tag antibodies will be used for Western blot analysis. MS-MS analysis was performed on isolated protein to give another validation for PrK incorporation.

### Enzyme Site-Specific Wiring to GCE

Ten μL of pyrene-azide clicked to PrK containing FGM was dropped on clean GCE and incubate in RT for 15 min. The pyrene groups from the linker adhere to GCE surface through π-π stacking. GCE was washed using DW to avoid unbound protein showing signal in electrochemical measurements.

Relevant FGM DNA Sequences (all Sequences Encode the FAD-GDH α-Subunit+MCD, without the γ-Subunit):

R42X (SEQ ID NO: 14); S247X (SEQ ID NO: 15); D395X (SEQ ID NO: 16); T558X (SEQ ID NO: 17); P560X (SEQ ID NO: 18), wherein X is a non-canonical amino acid (ncAA).

Relevant FGM protein sequences (all sequences are of the FAD-GDH α-subunit+MCD, without the γ-subunit): R42X (SEQ ID NO: 19); S247X (SEQ ID NO: 20); D395X (SEQ ID NO: 21); T558X (SEQ ID NO: 22); P560X (SEQ ID NO: 23), wherein X is a non-canonical amino acid (ncAA).

### Example 2

### Reagents and Materials

All DNA oligos used for the plasmid construction were purchased from Syntezza Bioscience Ltd. (Jerusalem, Israel). For the bacterial expression cultures, Auto-induction medium was purchased from Formedium™ (Hunstanton, England), glycerol was purchased from Bio-Lab ltd. (Jerusalem, Israel), chloramphenicol was purchased from Chem-impex int'l (Wood Dale, IL, USA), carbenicillin was purchased from Apollo (Manchester, England) and Propargyl-1-lysine was purchased from Synchem (Elk Grove Village, IL, USA). Tris buffer was purchased from Fisher scientific (Geel, Belgium), sodium chloride from Bio-Lab ltd. (Jerusalem, Israel), Imidzole from Glentham Life Sciences Ltd (Corsham, England), and Bradford reagent and Ni-NTA beads from Merck (Rehovot, Israel). Dimethoxybenzidine was purchased from Alfa Aesar (Heysham, England), 2,6-Dichlorophenolindophenol was purchased from Sigma-Aldrich (Rehovot, Israel), phenazine methyl sulfate and PCA from Tokyo chemical industry (Tokyo, Japan). TAMRA-Azide, THPTA and sodium ascorbate were purchased from Sigma-Aldrich (Rehovot, Israel). PDAz linker was purchased from Lumiprobe (Hunt Valley, MD, USA). Highly oriented pyrolytic graphite grade ZYB was purchased from Ted Pella inc. (Redding, CA, USA). Sodium acetate was purchased from Avantor inc. (Radnor, PA, USA), acetic acid from Bio-Lab ltd. (Jerusalem, Israel) and glassy carbon electrodes, Ag\AgCl reference electrodes, alumina polishing pad and 0.05 μm alumina slurry were purchased from ALS (Tokyo, Japan). Urea, ascorbic acid and lactate were purchased from Sigma-Aldrich (Rehovot, Israel).

### Protein Expression and Verification

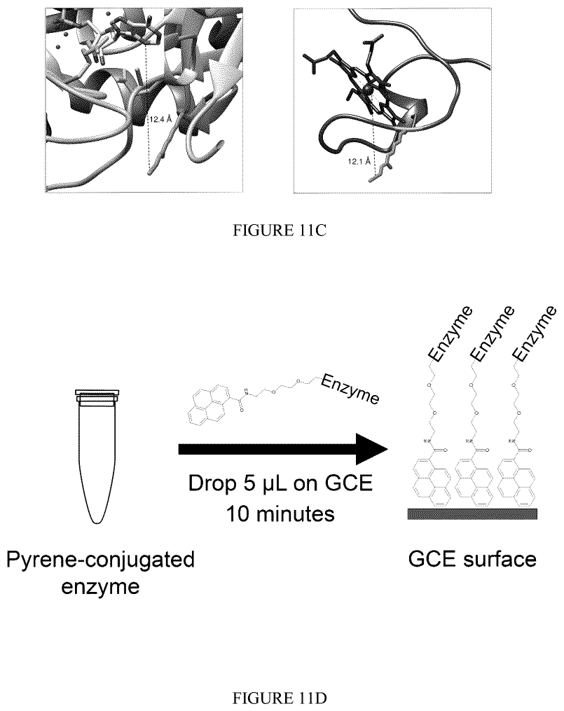

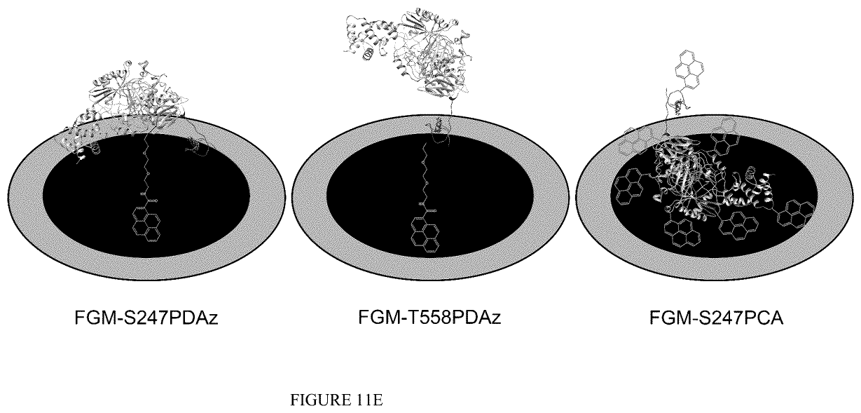

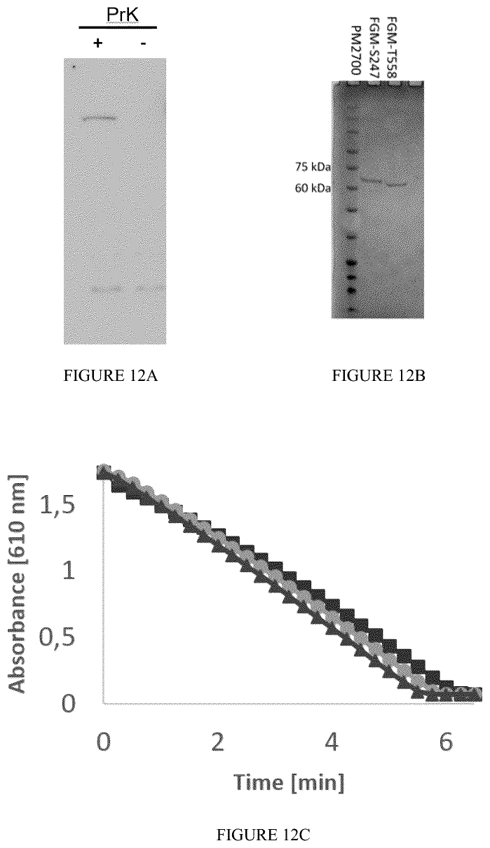

FGM-S247PrK (FIG. 11A) was expressed in 100 mL Auto-induction media supplemented with 1:100 overnight cultured bacteria, 50 μg/mL chloramphenicol, 100 μg/mL carbenicillin, 0.5% glycerol and 2 mM of UAA Propargyl-1-lysine (PrK) (FIG. 11B (1)) for 48 h in 20° C. The cells were then collected using centrifugation in 8000 rpm for 10 minutes followed by two washes with 15 mL lysis buffer (20 mM tris pH=8.0, 20 mM Imidazole, 500 mM NaCl). Cells were lysed using sonication needle and lysates were centrifuged for 30 min at 11,000 rpm to get a clear lysate. The lysate was then purified using immobilized metal affinity chromatography purification beads. The eluted protein concentration was then estimated using a Bradford assay to be 1 mg/mL and verified using anti his-tag Western blot analysis (FIG. 12A), in-gel heme staining (FIG. 12B) and 2,6-Dichlorophenolindophenol (DCIP) glucose oxidation activity assay (FIG. 12C).

Verification of UAA Incorporation into the Protein Sequence

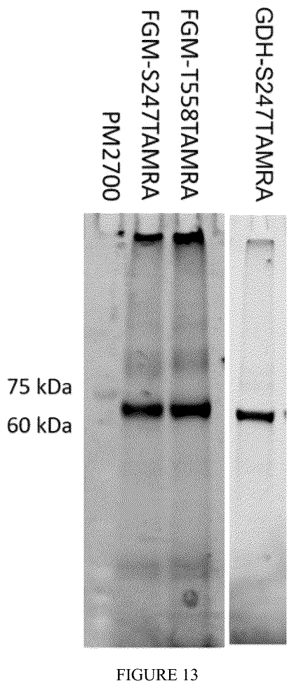

To verify UAA incorporation in response to the TAG mutation, a Cu(I)-catalyzed azide-alkyne cycloaddition (click reaction) was used to bind the alkyne residue of (1) to the azide residue of the fluorescent marker 5-Carboxytetramethylrhodamine-azide (TAMRA-Az). Six (6) 1 μM purified protein was mixed with 50 μM TAMRA-Az, 100 mM phosphate buffer (PB) pH=7.0, 200 μM CuCl₂, 1.2 mM Tris(3-hydroxypropyltriazolylmethyl) amine (THPTA) and 2.5 mM sodium ascorbate (NaAsc), and incubated for 1 h in the dark at RT with shaking. The fluorescence of the conjugated protein-TAMRA-Az was then analyzed by SDS-PAGE imaging using ImageQuant LAS4000 imager on a Cy3 mode (GE Healthcare, Little Chalfont, UK) (FIG. 13).

### In-Gel Heme Staining

Polyacrylamide gel after SDS-PAGE was first washed with DDW to remove the electrophoresis buffer. The gel then incubated in 20 mL DDW, and 5 mL of dimethoxybenzidine (DMB) solution were added to a final concentration of 0.8 mg/mL. After 10 minutes incubation with shake, hydrogen peroxide was added to a final concentration of 0.7% v/v and gel was incubated with shake until bands of the oxidized DMB have appeared. Gel image was taken using ImageQuant LAS4000 imager.

### FAD-GDH Glucose Oxidation Activity Assay Using DCIP

D-glucose oxidation was measured biochemically in the presence of 0.6 mM phenazine methyl sulfate (PMS), 0.6 mM 2,6-Dichlorophenolindophenol (DCIP), 100 mM D-glucose and 4.5 μM FGM-S247PrK solution in total volume of 80 μL. All dissolved in tris buffer pH=7.0. The 80 μL mix absorbance in 610 nm was measured using plate reader (BioTek instruments, Winoosky, VT) in 37° C. with shacking. Decrease in the DCIP absorbance indicates glucose oxidation. Enzyme activity unit was determined to be the amount of enzyme oxidizing one μmol of substrate per minute. The inventors have calculated the extinction coefficient of DCIP to be 4.7 cm⁻¹ mM⁻¹.

### AFM Analysis

To assess the protein wiring and its distribution on the electrode surface, a highly oriented pyrolytic graphite (HOPG) grade ZYB was used as the substrate for AFM measurements. 5 μL drop of 3 μM pyrene-conjugated protein sample was incubated on a freshly cleaved HOPG surface for 10 minutes followed by a wash with double distilled water (DDW), to remove unbound molecules, then drying in air before a measurement in 100 μL of 100 mM acetate buffer pH=5.0. HOPG surface was cleaned before each measurement by cleaving it and exposing a new layer using an adhesive tape. Measurements were performed using a Cypher-ES (Asylum research, Oxford instruments) on AC mode with a micro cantilever BL-AC40TS (Olympus, Japan).

### Verification of Protein Wiring on the Electrode

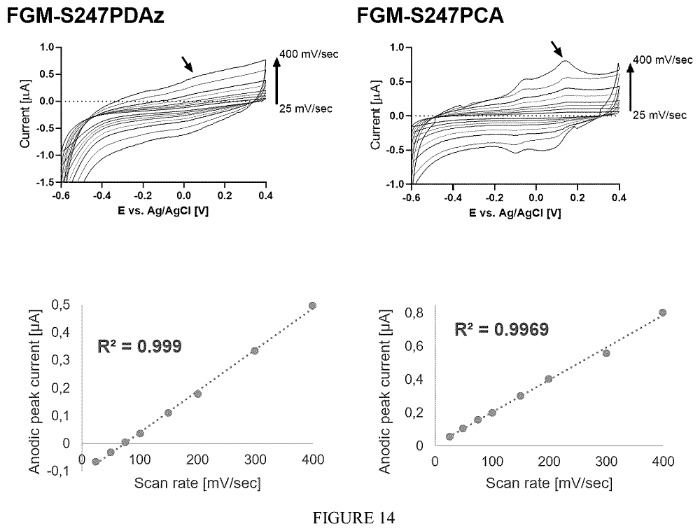

To verify that both FGM-S247PDAz and FGM-S247PCA are wired on the electrode surface, we first identified peaks using CVs (at 100 mV and 145 mV vs. Ag/AgCl, respectively). By using different scan rates, we have plotted the anodic peak current vs. the scan rates (FIG. 14). It can be seen that both samples showed high linearity of peak currents with the scan rate and not with its square root (R²>0.99) which indicates wired specie on the electrode surface.

UAA Incorporation into FGM

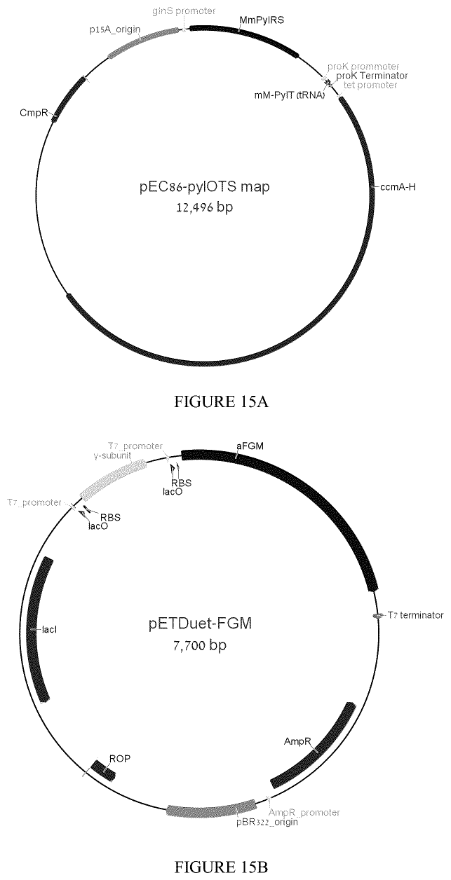

E. coli BL21 bacterial expression system was used for the expression of the FGM enzyme with site-specifically incorporated UAA. Pyrrolysyl orthogonal translation system (pylOTS) DNA sequence was amplified from the pEVOL plasmid using polymerase chain reaction (PCR) and cloned into the backbone of pec86 plasmid using Gibson's assembly to generate a new plasmid called pec86-pylOTS (FIG. 15A). pylOTS allows the continuous expression of an orthogonal translation system (orthogonal tRNA and aminoacyl tRNA synthetase) for incorporation of PrK (1) using the amber (TAG) stop codon suppression, while the pec86 plasmid is responsible for the continuous expression of E. coli cytochrome c maturation system. The second plasmid, pETDuet-FGM (FIG. 15B) was used for the expression of FGM with a TAG mutation encoded in the desired incorporation site. Four different TAG mutants were planned using site-directed mutagenesis, according to GDH crystal structure. Our guiding rational for mutation sites was to control the enzyme orientation in a manner that will allow proximity of 14 Å or less between the FAD or heme site and the electrode, without interference with the protein structure and correct folding. The sites that were tested were R42TAG and S247TAG, which are in close proximity to the FAD binding-site, while T558TAG (FIG. 11C, right panel) is in a close proximity to the heme domain. For the FAD proximity mutation site, we had the highest expression levels and activity with the S247TAG mutant (FIG. 11C, left panel). Hence, from here on we have focused our studies on S247TAG and T558TAG mutants. As a control that will allow us to find out whether the MCD affects ET properties of a site-specifically wired enzyme, we have also generated an S247TAG mutant GDH that lacks its MCD domain.

Protein Conjugation with Pyrene Containing Linkers

For the site-specific wiring of FGM-S247PrK, FGM-T558PrK and GDH-S247PrK the inventors have used a pyrene-diethyleneglycol-azide (PDAz) linker (FIG. 11B, 2). The azide in (2) was clicked to the alkyne residue of (FIG. 11B, 1). Three (3) μM of protein sample was mixed with 400 μM (2), 2 mM tris pH=8.2, 10 mM KCl, 1 mM Cu(I), 1.2 mM THPTA, 2.5 mM NaAsc and 10% Dimethyl sulfoxide (DMSO). The reaction was incubated for 1 h in the dark at room temperature with shaking, then transferred to 4° C. for two days before use. For the non-specific wiring of FGM-S247PrK we have used a standard EDC-N-Hydroxysuccinimide (NHS) coupling with a PCA linker (FIG. 11B, 3). 3 μM of protein sample was mixed with 200 mM EDC, 400 mM NHS, 25 mM 4-(2-hydroxyethyl)-1-piperazineethanesulfonic acid (HEPES) buffer pH=7.0 and 100 μM (3) and incubated for 12 h at RT with shaking.

### Electrode Preparations and Electrochemical Measurements

Glassy carbon electrode was first polished on 0.05 μm alumina polishing pad followed by five minutes sonication in DDW and drying using pure argon gas. Protein wiring to electrode was performed by dropping 5 μL of 3 μM pyrene-conjugated protein sample on GCE surface for 10 minutes incubation at RT (FIG. 1D) to allow the wiring of the protein through pi-pi stacking interactions between the pyrene groups and the GCE surface. After 10 minutes, the electrode was rinsed with DDW to remove unbound molecules and measured using a standard 3-electrodes electrochemical system with Ag/AgCl reference electrode and a pencil rod as the counter electrode. The expected orientations of all FGM variants towards the electrode are presented in FIG. 11E. GDH-S247PDAz is not presented since it is expected to be oriented like FGM-S247PDAz, lacking MCD. All measurements were performed using Palmsense 3 potentiostat (Palm Instruments, Houten, The Netherlands) at RT in 100 mM acetate buffer pH=5.0 under argon atmosphere and at variable scan rates. DPV measurements were performed with potential pulse of 100 mV for 0.1 sec, with scan rate of 25 mV/sec. In the multistep amperometry measurements the electrode's potential was biased from (−200) mV to 0 mV, framing the onset potentials of all modified electrodes, measuring the current decay for 10 sec for each step. In case of negative current values, a constant value was added to allow current decay linearization using natural logarithm (1n) transformation. To test the enzyme-electrode performance in the presence of interfering molecules, we have used acetate buffer supplemented with 50 μM ascorbic acid, 10 mM lactate and 10 mM urea (ALU) and measured the current response upon application of 150 mV vs. Ag/AgCl reference electrode.

### Results and Discussion

### Enzyme Characterization

FGM-S247PrK expression was first verified using anti His-tag western-blot analyses (FIG. 12A). Two expression cultures were grown, while only one of them was supplemented with 2 mM of (1), the cells were then lysed and analyzed. It can be seen that only with the supplementation of (1) to the expression culture we could observe a 67 kDa band indicating the successful expression of a full-length His-tagged protein. In the absence of (1) in the growth culture, we could not detect any protein band in the relevant size. The incorporation of (1) into the protein was verified by “clicking” a tetramethylrhodamine-Azide (TAMRA-Az) to the FGM-S247PrK, GDH-S247PrK and FGM-T558PrK protein variants. Fluorescent gel image (FIG. 13) has demonstrated that the mutants of FGM and GDH indeed had (1) incorporated into their amino-acid sequence. One band in the expected size indicated that the FGM/GDH mutants are the only proteins in the sample that contain the alkyne residue of (1). Next, the inventors have used in-gel heme staining to verify a proper minimal cytochrome c maturation process (FIG. 12B), where a band of around 67 kDa was observed, indicating a successful heme maturation. The enzyme's catalytic activity towards glucose was tested biochemically using 2,6-Dichlorophenolindophenol (DCIP) colorimetric assay for glucose oxidation (FIG. 12C). The colorimetric activity assay indicated that FGM-S247PrK, GDH-S247PrK and FGM-T558PrK are all capable of oxidizing glucose with a similar specific activity.

### AFM Measurements

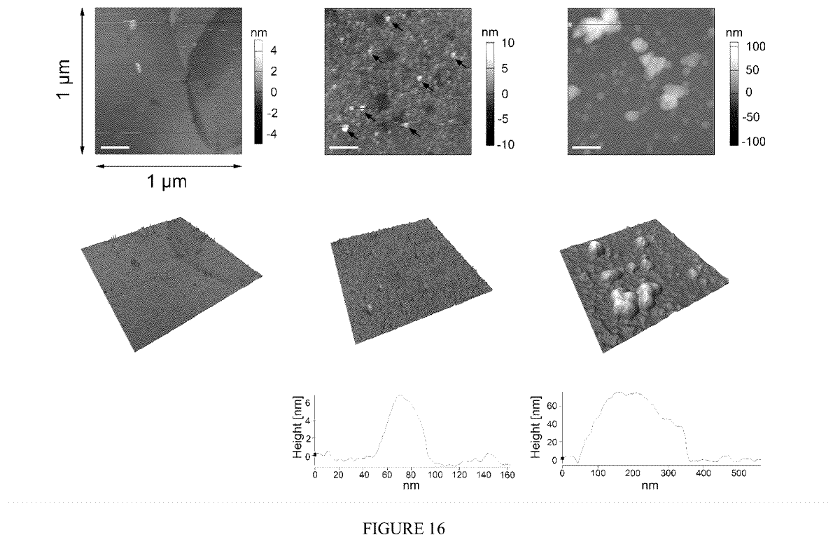

Understanding protein orientation on carbon surface can be realized by the combination of the knowledge the inventors have from the protein crystal structure (FIG. 11A) with AFM measurements. The inventors have analyzed the topography of an atomically flat carbon surface after wiring FGM-S247 in different methods and compare it to the known expected size of the protein. FIG. 16 shows the AFM measurements of HOPG surfaces after 10 minutes of incubation in acetate buffer (FIG. 16, left panel), FGM-S247 wired through (2) (FIG. 16, middle panel) and FGM-S247 wired through (3) (FIG. 16, right panel). Since HOPG is atomically flat, the inventors could measure the height of the bound enzyme on the HOPG surface and compare it to an approximate enzyme foot-print and height calculated from the crystal structure of FAD-GDH (FIG. 11A) taking into account the estimated lengths of the respective linkers. As a control, only acetate buffer added to the surface, does not result in salt accumulation (FIG. 16, left panel). Measurement of the FGM-5247 linked through (3) showed islands of bound molecules averaging around 70 nm in height, almost 10 times higher than the height expected from a monolayer of FGM. These islands may be a result of protein aggregation caused by the wiring of the enzyme through multiple sites as expected from non-specific wiring. Coupling to (3) may result in multiple pyrene molecules on each protein. The adhesion of the protein to the surface through multiple pyrene residues can expose the hydrophobic core of the protein, which may result in the aggregation observed in the AFM measurements. However, measurement of the FGM-5247 clicked to (2) showed scattered particles on the surface with an average height of ca. 6 nm. When taking into consideration the anchoring point of the protein (S247) and the length of (2) (which is estimated to be 0.6 nm in its stretched form), this observation is very close to the estimated height of a single layer of a wired FGM in its expected orientation (FIG. 11A) on the HOPG surface. These results suggest that using site-specific wiring affords a relatively good control over the protein orientation towards the surface as well as better surface coverage of active and correctly folded enzyme with a mono-layer pattern, although not a densely packed one, rather than multi layered and partially unfolded aggregates.

### Electrochemical Characterization

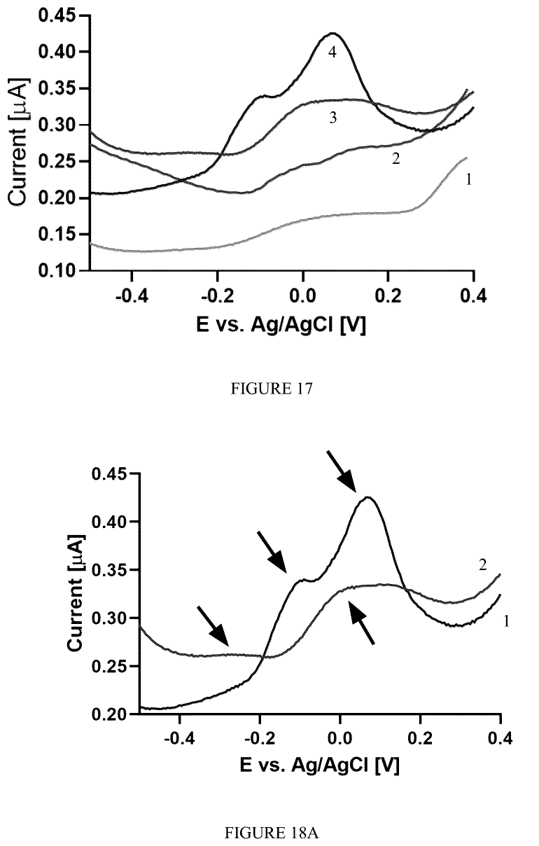

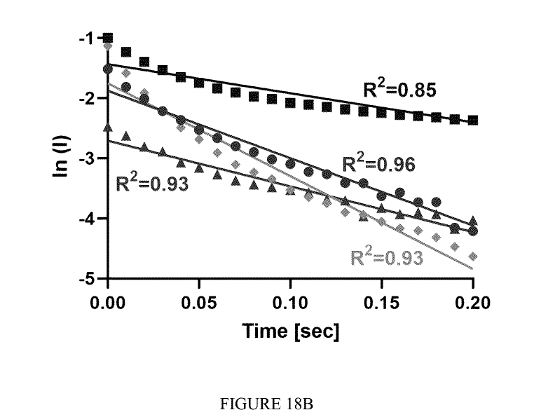

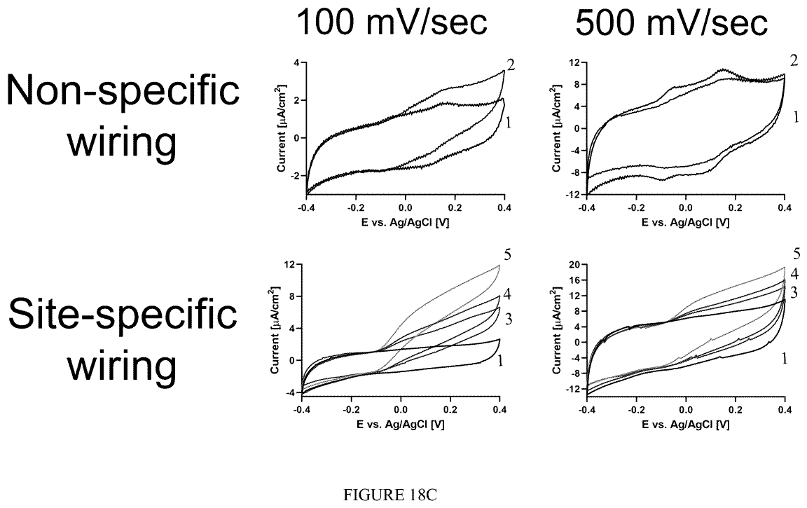

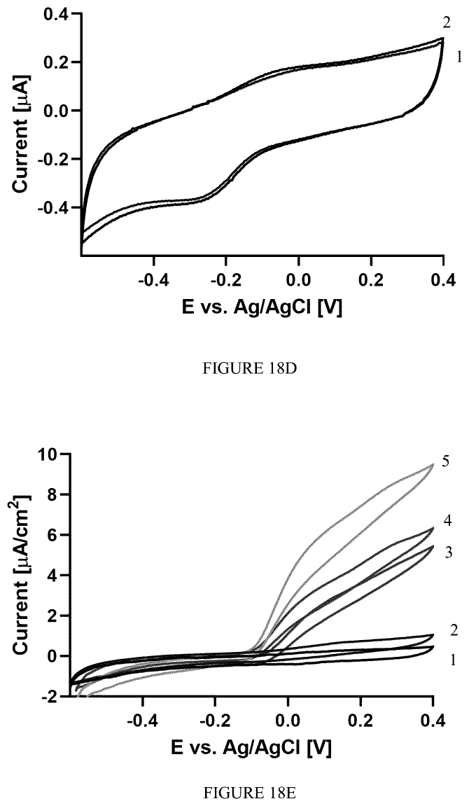

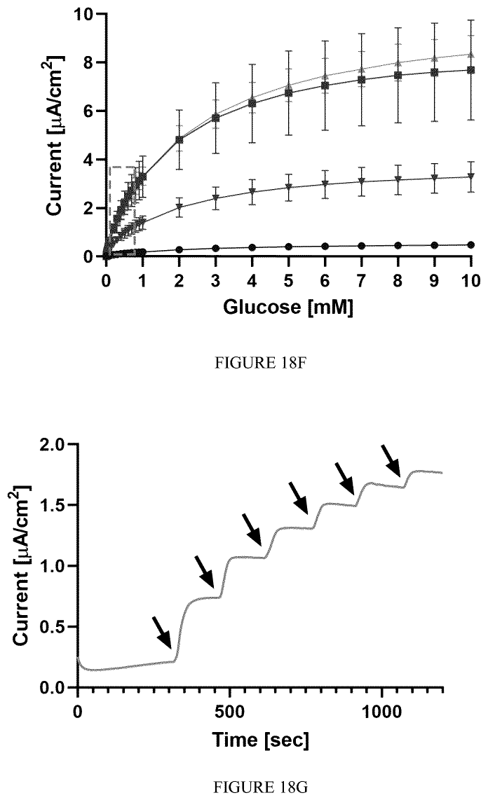

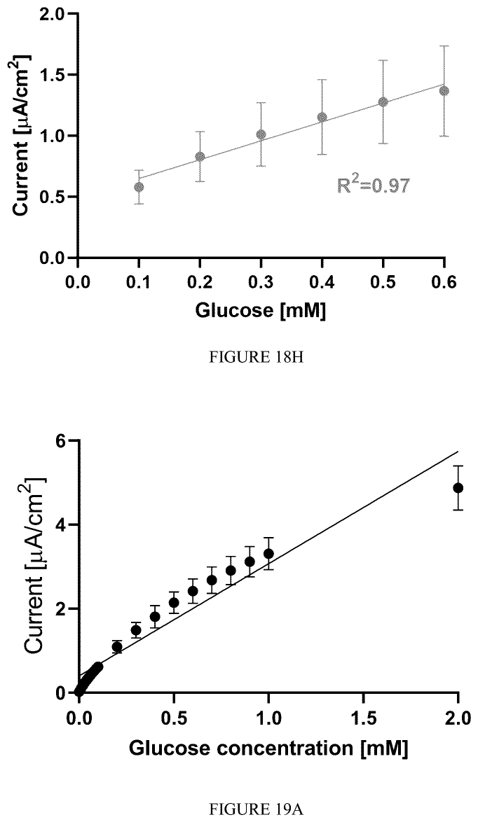

For the electrochemical characterization of the wired enzyme, three different enzyme variants were compared using a site-specific wiring approach with the PDAz linker. In addition, the site-specific wiring approach was compared to a non-specific wiring approach using EDC coupling of one of the mutants to the electrode (FGM-S247PCA). In order to see whether the electroactive peaks can be detected and differentiated between the wiring approaches, the inventors have conducted DPV measurements that are more sensitive and less prone to background from non-Faradaic currents. FIG. 18A shows DPVs of FGM-S247PDAz and FGM-S247PCA in the absence of glucose. While FGM-S247PCA shows two well-defined peaks at (−100) mV and (+75) mV, FGM-S247PDAz shows a minute peak at (−250) mV and a defined albeit wide peak at ca. 0 mV vs. Ag/AgCl reference electrode. A control experiment where the DPV was performed on the conjugation reagents only (without adding protein samples) shows that the observed peaks indeed originate from FGM electroactive-sites (FIG. 17) and not from the presence of other factors in the reaction mixture.

In order to elucidate the source of the two defined peaks in the non-specifically bound enzyme compared with the broad peak in the site-specifically bound enzyme, the inventors have calculated the apparent ET coefficient (k_{ET}^{app}) using multistep amperometry as was previously described by the inventors. In FIG. 18B it can be seen that the linearized current decay of the site-specifically wired enzyme has a mono-exponential decay characteristic (R2>0.93) while the non-specifically wired enzyme has a bi-exponential decay dependence, which indicates ET from one or two redox active-sites. It cannot be excluded that the observed mono-exponential characteristic signal is an average from both, FAD and heme domain, but it can be deduced from this result that only one orientation is dominant on the electrode surface. Hence, the inventors could extract k_{ET}^{app} values from the slope of the linearized current decay curve as summarized in Table 3.

TABLE 3
Apparent electrochemical constants of the different tested variants

| Enzyme variant | K_{M}^{app} (mM) | iₘₐₓ (μA cm⁻²) | k_{ET}^{app} (s⁻¹) |
|---|---|---|---|
| GDH-S247PDAz | 1.55 ± 0.16 | 3.74 ± 0.12 | 8.0 ± 0.6 |
| FGM-S247PDAz | 1.89 ± 0.10 | 8.88 ± 0.40 | 11.2 ± 0.7 |
| FGM-T558PDAz | 1.58 ± 0.22 | 9.80 ± 0.18 | 13.4 ± 1.4 |
| FGM-S247PCA | 1.82 ± 0.18 | 0.55 ± 0.02 | 4.80 ± 0.02 |

Since the inventors have observed a mono-exponential current decay from the site-specifically bound enzymes variants and a bi-exponential current decay from the non-specifically bound one, it could be an indication of the source of the two peaks: whereas one peak origin is a direct communication between the heme and the electrode and the other is a mediated ET from the FAD to the heme and then to the electrode. The inventors hypothesize that the porphyrin, in the case of non-specific attachment is adsorbed directly on the electrode due to two lysine residues in a close proximity to its binding site. Whereas an internal ET (between the FAD and the heme) can be hardly observed when fast scan rates are being used as no electrocatalytic current is apparent in the presence of glucose under higher scan rates regime and only the two separate peaks are visible and are well defined (which is an indication of Faradaic processes but not catalytic ones) (FIG. 18C). This result is in agreement with a control experiment that have shown that no electrocatalytic current is observed from attaching the heme binding domain only to the electrode in the presence of glucose as it cannot oxidize glucose in the absence of the FAD binding domain (FIG. 18D).

FIG. 18E shows the bioelectrocatalytic currents of three site-specifically wired variants and one non-specifically wired enzyme in response to 5 mM glucose addition using CV with a slow potential scan rate of 10 mV/sec. As opposed to a high-potential-scan rate measurements (FIG. 18C), at a low scan rate, the inventors observed the catalytic currents from the different variants almost independently from ET rates (provided that those ET rates are faster than the potential scan rate). The catalytic response of the site-specifically wired enzymes was much higher compared to the non-specifically wired enzyme (ca. 10 folds higher increase in maximal currents) and its onset potential is lower (−100 mV for all the site-specifically wired enzymes compared to −45 mV for FGM-S247PCA). This result indicates better accessibility of the protein's electroactive site towards the electrode. Within the site-specifically wired enzymes, both GDH-S247PDAz and FGM-S247PDAz, which are bound with proximity to the FAD domain, present almost the same catalytic current while the FGM-T558PDAz shows almost two-fold higher catalytic current compared to both of them. In the site-specifically wired enzyme the substrate has a direct access to the catalytic site and the electrons can be transferred directly to the electrode without any barriers. By comparing the two points of attachment it was evident that higher catalytic currents are observed when the enzyme was wired close to its cytochrome domain (FGMT558TAG) rather than through sites that are close to its FAD binding-site (FGMS274TAG). While with the non-specifically wired enzyme, ET can be deterred by proteins covering the electrode in different orientations that are not optimal for efficient ET.

For a given glucose concentration, the highest catalytic current was observed for FGM-T558PDAz variant, followed by FGM-S247PDAz and the lower catalytic current was observed with the GDH-S247PDAz electrode. Using high potential scan rates can help us understand whether the MCD has a role in the electron transfer to the electrode. While using low potential-scan rates resulted in almost the same catalytic current for both GDH and FGM, which are wired through the 5247 site, using high scan rates resulted in higher currents for FGM. This observation suggests that the MCD participates in the ET process to the electrode, rendering it more efficient.

Apparent electron transfer rate constants were determined for the different variants, the highest k_{ET}^{app} was observed for FGM-T558PDAz wired to the electrode close to its MCD, next was the FGM-S247PDAz wired close to its FAD binding-site, the lowest value was that of GDH-S247PDAz which is wired in a close proximity to its FAD however in the absence of MCD and the lowest value was that of the non-specifically wired FGM-S247PCA. Glucose concentration vs. current calibration curves of all variants is presented in FIG. 18F. It can be seen that the site-specifically wired enzymes show much higher currents than the non-specific wired ones. The apparent electrochemical Michaelis-Menten constants were determined based on a non-linear fit of the calibration curves shown in FIG. 18F (Table 3). K_{M}^{app} of all samples did not differ significantly between variants within the error of the measurements in the range of 1.55-1.89 mM which is an indication that the mutagenesis and the site of UAA incorporation did not modify the enzyme affinity to glucose. However, iₘₐₓ values were calculated to be more than 15 times higher for FGM-S247PDAz with 8.88±0.40 μA cm⁻² vs. 0.55±0.02 μA cm⁻² for FGM-S247PCA. The site of attachment has also an effect on the iₘₐₓ values. Wiring through the T558TAG site with a proximity to the heme domain resulted in the highest current value, a slightly lower value was that of the S247TAG variant and the lowest value was that of the GDH-S247TAG. This result is another indication that the MCD has an effect on the ET abilities of the protein and are in correlation with the calculated k_{ET}^{app} values. In comparison with iₘₐₓ value previously reported by the present inventors for non-wired FGM, which was 2.0±0.5 μA cm⁻², the site-specific wiring demonstrates more than four times higher iₘₐₓ value which is a significant improvement. Those high currents allow much higher resolution for glucose biosensing with higher sensitivity, which could be very useful and physiologically relevant for samples with low glucose concentrations such as in sweat, subcutaneous plasma or tears samples. The limit of detection (LOD) was found to be 10 μM glucose for the site-specifically wired enzymes compared to 100 μM for the non-specifically wired enzyme, with a signal to noise ratio that is larger than 3. Since FGM-T558PDAz showed the highest currents in response to glucose, the inventors have presented its current response to the physiologically relevant concentrations in tears and sweat in the inset of FIG. 2(F). FGM-T558PDAz present a resolution of 10 μM glucose and high linearity (R²=0.988) of the current measured in the 0.01 to 0.6 μM of glucose. The broader linear range of FGM-T558PDAz was found to be 0.01-2 mM glucose while FGM-S247PCA showed a linear range of 0.1-2 mM only (both with R²>0.92) (FIGS. 19A-19B). Improving the surface coverage will allow more available reaction sites (in a similar manner as improving enzyme specific activity, this could be achieved by using porous electrodes for example) that can result in a broader dynamic range. To test the herein disclosed wired enzyme response in the presence of interfering molecules, the inventors have used known interfering molecules in tears samples as previously described. The current response of FGM-T558PDAz was tested in ALU solution upon the addition of 0.1 mM glucose increments (FIG. 18G). The current response in the physiologically relevant glucose concentrations in tears (0.1-0.6 mM) was found linear under these conditions as shown in FIG. 18H, no interference was observed under these conditions. Being able to detect glucose in the relevant concentrations without being interfered by the ALU solution makes our system suitable for glucose detection in bodily fluids other than blood.

In conclusion, the inventors have presented the importance of a redox enzyme orientation towards an electrode. Non-specific wiring methods such as EDC-NHS coupling allows the covering of GCE with proteins but without the ability to control its orientation. By using site-specific UAA incorporation, the inventors have created a unique orthogonal “chemical handle” that allows the conjugation of a linker in only one anchoring point on the protein sequence, and by that allowed determination of a specific orientation towards the electrode. The controlled orientation results in ca. 20 times higher catalytic currents in response to glucose, higher ET efficiency that was shown by the ability of the enzyme to transfer electrons in a scan rate as high as 500 mV/sec and a scattered mono-layer pattern on the surface as observed by AFM measurements. Wiring of proteins through different sites result in a significant effect on their ET characteristics and their ability to communicate with an electrode. Using this method allows high flexibility in the UAA incorporation site which makes it a powerful tool for advanced protein engineering for enzyme-based biosensors. When site-specifically orienting a glucose oxidizing enzyme on the surface of an electrode, the inventors could observe a gain in sensitivity (down to a concentration of 10 μM glucose), however this gain in sensitivity comes with a cost, in gaining sensitivity the inventors have lost the dynamic range that allows for higher glucose concentrations sensing. Testing FGM enzyme-electrode in ALU solution demonstrated no interference when using known interfering molecules present in tears. This makes the enzyme more suitable for non-invasive biosensors for bodily fluids other than blood such as sweat, tears and urine.

### Example 3

### FGM Expression Improvement

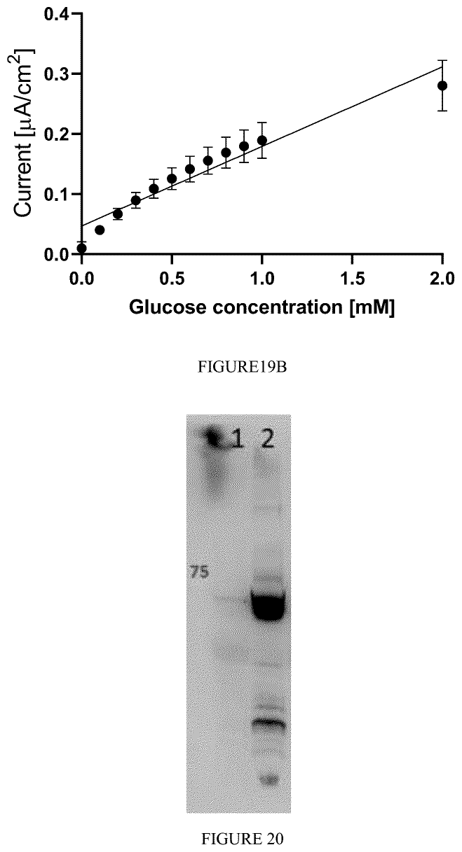

FGM expression gene is built from two sub-units, the alpha catalytic subunit, and the gamma subunit which is a helper protein that enables correct folding of the alpha subunit and responsible for the high stability of the whole protein complex. So far, the inventors have been working with an expression plasmid that has both genes under the same promoter (Trc promoter), leading to low expression levels of FGM. In order to improve its expression levels, the inventors have cloned the FGM gene into a pETDuet plasmid, where each of the subunits is expressed from “its own” T7 promoter. The use of pETDuet results in much higher expression levels for FGM (FIG. 20), indicating that using a different promoter for each subunit indeed increases expression efficiency.

### Site-Specific Wiring of FGM to an Electrode

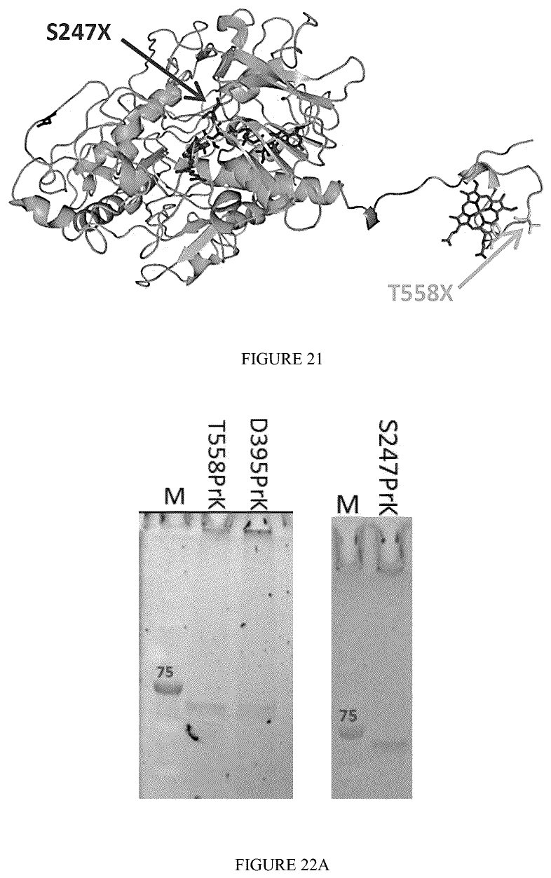

The structure of FAD-GDH from Burkholderia cepacia was published recently and enabled the inventors to better understand the structure of the protein and better plan ncAA incorporation sites for site-specific wiring to an electrode. 5247 is estimated to be 9.9 Å from the FAD site and T558 is estimated to be 10 Å from the heme site (FIG. 21). Using those variants allow site-specific ‘wiring’ to an electrode with high proximity to allow direct electron transfer. To allow the incorporation of ncAA along with heme maturation, the inventors have engineered the pec86 plasmid (cytochrome c maturation plasmid) by fusing into its backbone the orthogonal tRNA and aminoacyl tRNA synthetase DNA sequences from pEVOL-pylOTS plasmid (ncAA incorporation plasmid), creating a new plasmid—pEC86-pylOTS.

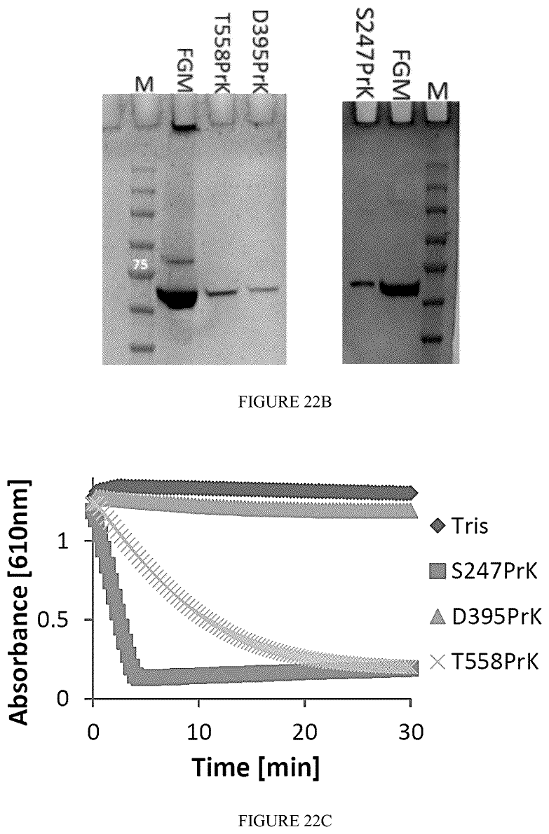

Using the new ncAA incorporation and heme maturation plasmid (pec86-pylOTS) the inventors have managed to express the FGM ncAA variants with a mature c-type cytochrome. The incorporation of propargyl-lysine (PrK) was verified using a click reaction to a fluorescent marker (TAMRA-Azide) and the heme maturation was verified using in-gel heme staining. The glucose oxidation activity of the different variants was tested using a standard FAD-GDH activity assay (FIGS. 22A-22C).

The ‘click’ reaction with TAMRA-Azide has proved that the inventors system successfully expressed FGM with PrK incorporation (FIG. 22A). In-gel heme staining approved that our variants have a matured c-type cytochrome and that our pec89-pylOTS plasmid can do both, ncAA incorporation and heme maturation (FIG. 22B). Using the FAD-GDH activity assay proved that the inventors variants are active and can oxidize glucose (FIG. 22C). The difference in the activity rate between S247PrKFGM and T558PrKFGM is probably due to the difference in protein concentrations. Different ncAA locations have been shown to affect the protein expression efficiency and it correlates with the bends' intensities in the protein gels (FIG. 22A, 22B).

D395PrKFGM was barely active, probably due to an effect of the change on the enzyme active site. The D395 site mutation was planned according to the SWISS-model homology and found to be different from the real protein structure (found in a surface exposed loop in the homology model, while the real protein structure showed that it is located in the middle of an important α-helix).

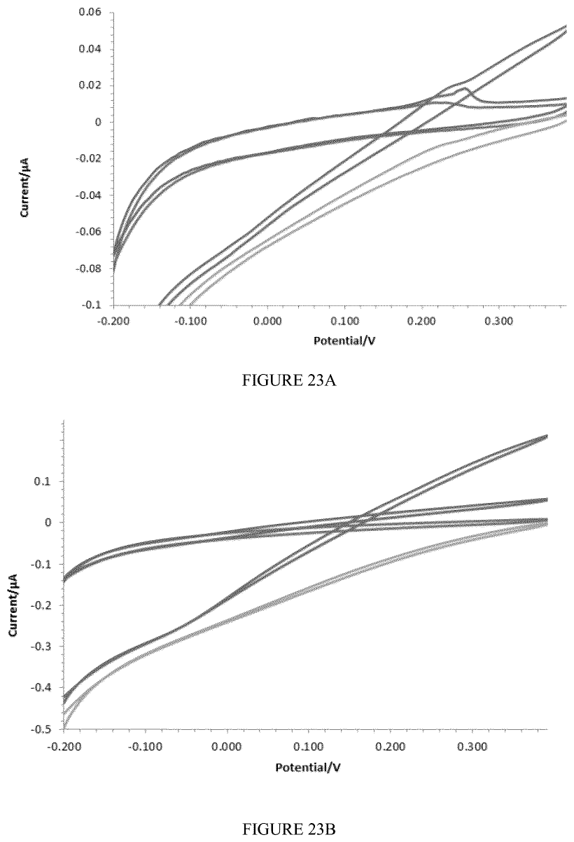

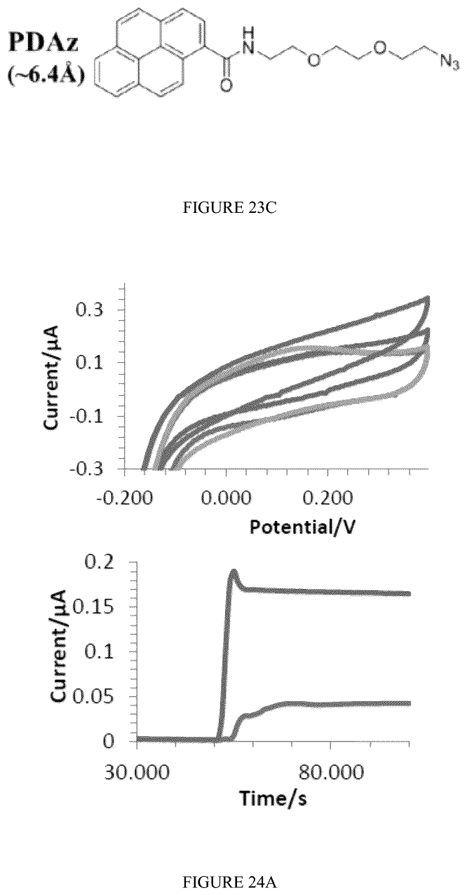

In the next step the inventors ‘clicked’ the different variants to a pyrene-azide linker (FIG. 23C) to test its site-specific ‘wiring’ to a glassy carbon electrode (GCE).

The inventors performed a click reaction on S247PrK FGM and on WT FGM to verify their site specific wiring. The inventors expected that wiring FGM will not work since it doesn't have an incorporated PrK, while S247PrKFGM will be wired to the electrode. From the results (FIG. 23A) it could be seen that S247PrKFGM was successfully wired to the electrode and allowed high catalytic currents in response to glucose addition while WT FGM showed no bioelectrocatalytic current. As opposed to S247PrKFGM, WT FGM couldn't stay on the electrode surface and was washed away in the measurements' buffer. When comparing wired and entrapped S247PrKFGM (FIG. 23B), it can be seen that the wiring of the protein allows higher catalytic currents and lower onset potential, indicating highly efficient electron transfer (ET) that is gained due to the site-specific wiring. Same tests were performed on T558PrKFGM variant, and it has been proved to be wired as well.

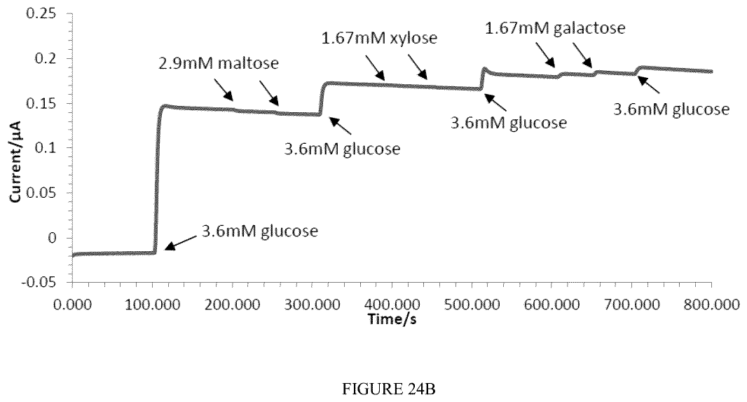

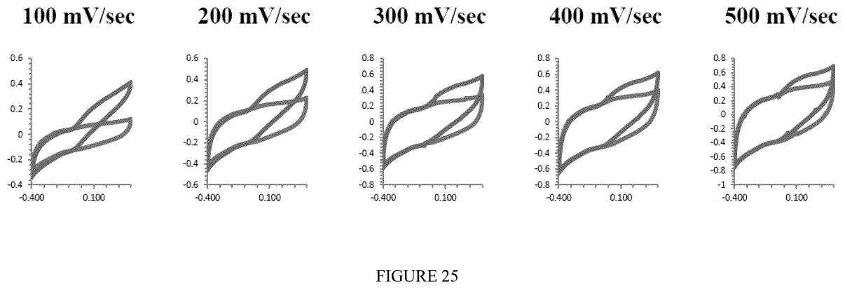

To eliminate the possibility that the click reaction reagents have affected the 247PrKFGM structure in a way that will allow higher catalytic response, the inventors have performed the click reaction without the pDAz linker (FIG. 24A). It was found that the clicked enzyme in the presence of pDAz showed higher catalytic current with lower onset potential indicating that the increase in efficiency is due to the site-specific wiring of FGM. The wired enzymes have been tested for electrochemical communication with the electrode in high scan rates (100-500 mV/sec) and showed catalytic currents even in those scan rates, indicating very fast ET rate (FIG. 25). The specificity of S247PrKFGM towards glucose was tested as well by introducing other sugars and measuring the chronoamperometric response (FIG. 24B). It has been shown that the wired enzyme didn't lose its specificity towards glucose.

### Example 4

### Electrochemical Characterization of a Dual Cytochrome-Containing Lactate-Dehydrogenase

### Materials and Reagents

Takara PrimeStar GXL DNA Polymerase was purchased from Takara (Saint-Germain-en-Laye, France), Dimethoxybenzidine (DMB), 2,6-dichloroindophenol (DCIP), and L-(+)-lactic acid were obtained from Sigma-Aldrich (Rehovot, Israel). Hydrogen peroxide (30%; v/v) and Coomassie protein assay reagent were purchased from Thermo Fisher Scientific (Modiin, Israel), nickel-nitrilotriacetic acid (NTA) agarose for protein purification was bought from Macherey-Nagel (Duren, Germany), and all DNA oligonucleotides were acquired from Syntezza Biosciences (Jerusalem, Israel). Screen-printed electrodes (SPE) DRP-110CNT were purchased from Metrohm DropSens (Asturia, Spain).

Plasmids and Sequences

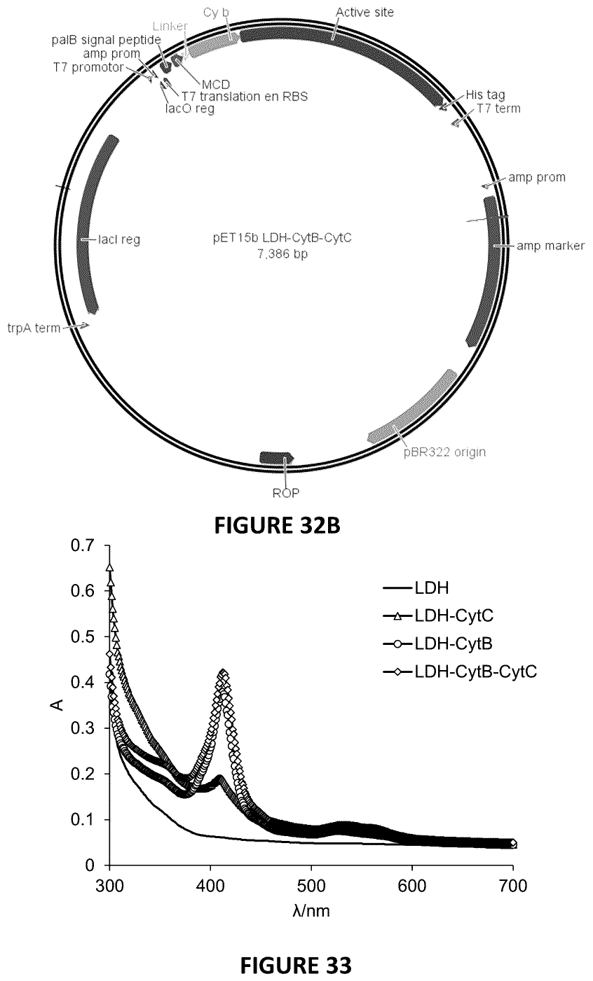

Complete details of the sequences and plasmid maps are provided in Table 4 below, and FIG. 32.

TABLE 4
Primers used in this study

| Primer | | | |
|---|---|---|---|
| number | Aim | Primer sequence | SEQ ID NO: |
| 1 | LDH-CytB-CytC: PelB | GCTGCCGACCGCTGCTGCTGGTC | 27 |
| | insertion, CytC amplification, | TGCTGCTCCTCGCTGCCCAGCCG | |
| | forward | GCGATGGCCATGATTCGTGCAGG | |
| | | TGCTACCATG | |
| 2 | LDH-CytB-CytC: PelB | GGAGCAGCAGACCAGCAGCAGC | 28 |
| | insertion, vector amplification, | GGTCGGCAGCAGGTATTTCATGG | |
| | reverse | TATATCTCCTTCTTAAAG | |
| 3 | LDH-CytB-CytC: CytC | CGGACCCGGCGGACCAGAGCCA | 29 |
| | amplification, reverse | TAACCAGAACCGAATTCCTGGAT | |
| | | AATAGCGTGACATG | |
| 4 | LDH-CytB-CytC: LDH-CytB | GAATTCGGTTCTGGTTATGGCTC | 30 |
| | gene amplification, forward | TGGTCCGCCGGGTCCGGAGCCG | |
| | | AAACTGGATATGAATAAAC | |
| 5 | LDH-CytB-CytC: LDH-CytB | TGCATCCTCAAATTCTGTTAAAG | 31 |
| | amplification, reverse | | |
| 6 | LDH-CytB-CytC: vector | GGACCTACTTTAACAGAATTTGA | 32 |
| | amplification, forward | GGATGCAGGCAGTGGTTCCGGC | |
| | | CATCAC | |
| 7 | LDH-CytC, forward | GGTGAAACTAAGGAAGATATC | 33 |
| 8 | LDH-CytC, reverse | CTTTTCTAGCGATATCTTCCTTAG | 34 |
| | | TTTCACCCGGACCCGGCGGACCA | |
| | | GAGC | |
| 9 | LDH-CytB, forward | CTGCTGCTCCTCGCTGCCCAGCC | 35 |
| | | GGCGATGGCCATGGAGCCGAAA | |
| | | CTGGATATGAATAAAC | |
| 10 | LDH-CytB, reverse | GCGGGCGAAATCTTTTGTTTATT | 36 |
| | | CATATCCAGTTTCGGCTCCATGG | |
| | | CCATCGCCGGCTGGGCAGC | |
| 11 | LDH, forward | GCTGCTCCTCGCTGCCCAGCCGG | 37 |
| | | CGATGGCCATGGGTGAAACTAA | |
| | | GGAAGATATCGCTAGAAAAG | |
| 12 | LDH, reverse | GTTCTTTTCTAGCGATATCTTCCT | 38 |
| | | TAGTTTCACCCATGGCCATCGCC | |
| | | GGCTGGGC | |

Removal of an S. cerevisiae Mitochondrial Pre-Sequence of the LDH

The native enzyme LDH from S. cerevisiae includes a signal peptide pre-sequence at its N-terminal that is meant to direct the enzyme to the yeast mitochondria where it properly folds and the pre-sequence is truncated, maintaining that sequence may result in misfolding of LDH when expressed in Escherichia coli (E. coli), hence it should be removed.

### Protein Expression, Purification, and Characterization

E. coli BL21 cells were used to express the enzymes used in this study. The expression plasmids pET15b, harboring genes of the relevant enzyme variant, and pec86, which contains the ccmABCDEFGH gene cassette for efficient expression of mature c-type cytochromes in E. coli, were transformed into E. coli BL21 using a standard heat shock transformation protocol. An overnight bacterial liquid culture was diluted 1:100 in auto-induction medium supplemented with 100 μg/mL carbenicillin, and 50 μg/mL chloramphenicol for selection and grown for 48 hours at 20° C. After cell growth to an optical density (O.D._{600 nm}) of 1.1, the culture was centrifuged at 4° C., 8,000 rpm for 10 minutes, and washed with lysis buffer (0.3 M NaCl, 50 mM Tris-HCl, pH 8). The sample was lyzed by eight minutes sonication on ice (30 seconds pulse on, 45 seconds pulse off at a 40% amplitude) in the presence of a protease inhibitor, an endonuclease, and lysozyme. To separate the supernatant, the sample was centrifuged for 30 minutes at 10,000 rpm at 4° C. The supernatant was filtered with a 0.22 μm syringe filter and purified using nickel-NTA histidine-binding resin. Imidazole (250 mM) was used for elution of the His-tag containing protein from the column. Excess imidazole was removed by dialysis against storage buffer (0.3 M NaCl, 50 mM Tris-HCl, pH 8). As storage at −80° C. was not suitable for storage of those proteins for the constructs containing MCD, the purified enzymes were stored at 4° C. and used as soon as possible.

### In-Gel Heme Staining

Polyacrylamide gels were washed and kept in 20 mL of double distilled water (DDW). A dimethoxybenzidine (DMB) solution (5 mL) was added to a final concentration of 0.8 mg/mL. The solution was acidified to better dissolve the DMB, and the gel was incubated for 10 minutes at room temperature. Hydrogen peroxide (600 μL of a 30% (v/v) solution) was added to achieve a final concentration of 0.72% (v/v) and the gel was shaken at room temperature until stained bands appeared.

### Determination of Enzyme Concentrations and Heme Incorporation and Maturation

The molecular masses and purification efficiency of the enzymes were verified on SDS-PAGE gels treated with Coomassie stain and by western blot using anti-histidine antibodies. Proper incorporation of heme and its maturation were verified by in-gel heme staining. The concentrations of the purified enzymes were determined by a combination of the standard Bradford assay and by assessing cytochrome absorbance at λ=410 nm (ε=106 mM⁻¹ cm⁻¹).

### Kinetic Measurements in Solution

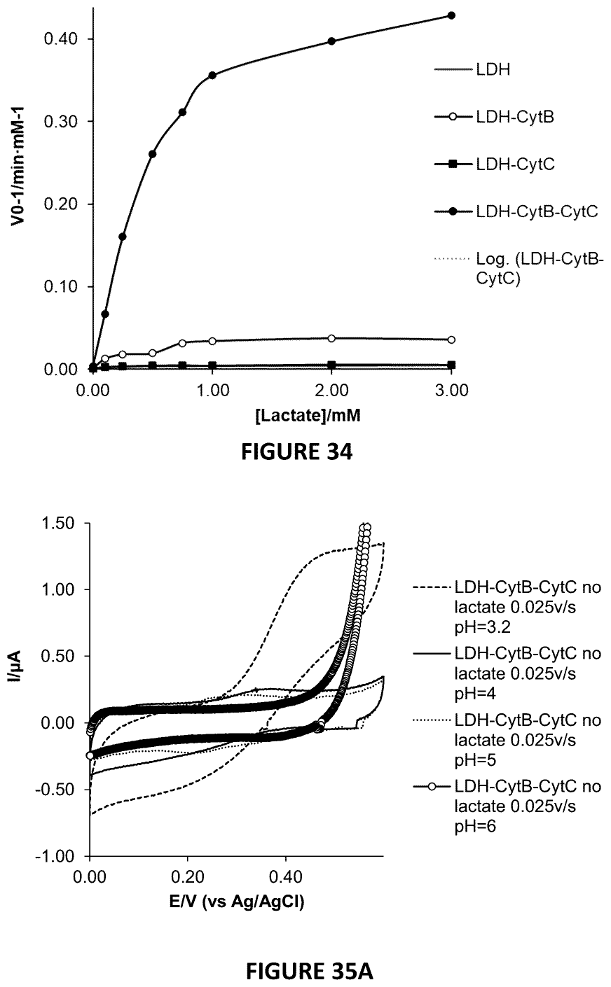

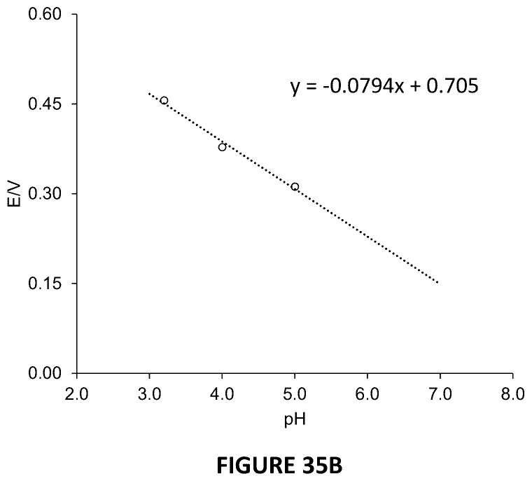

All tests of enzymatic activity followed the disappearance of 2,6-dichloroindophenol (DCIP) (ε=11.8 mM⁻¹ cm⁻¹) at λ=600 nm using a Synergy H1 plate reader (Lumitron, Petah Tikva, Israel). The reaction mixture contained 0.3 mM DCIP, 2 μM enzyme, and varying concentrations of L-lactate. All reactions were performed in 50 mM Tris-HCl, pH7.2 at room temperature and absorbance was normalized to the same initial DCIP concentration. To calculate Michaelis-Menten constants, a series of reactions were performed using 0.1 mM to 3.0 mM lactate. Based on the results, Michaelis-Menten and Lineweaver-Burk plots were constructed (FIG. 34) and kinetic and thermodynamic constants were calculated. The kinetic parameters k_{cat} and K_{M} were determined using the non-linear regression analysis feature of GraphPad Prism 8.0 software.

### Electrode Preparation

Electrochemical measurements were performed using SPEs, unless otherwise indicated. Both the working and counter electrodes were made of graphite, with the working electrode being modified with multi-walled carbon nanotubes, and the reference electrode being a pseudo-Ag/AgCl electrode. A 20 μL aliquot of enzyme at a 0.1 μM concentration was incubated with the working electrode at room temperature until the enzyme had dried, at which point the electrode was ready for use.

### Electrochemical Characterization

All measurements were performed with a Sensit Smart potentiostat (PalmSens Instruments, Houten, The Netherlands) in 100 mM phosphate buffer, pH 7.3, unless otherwise indicated. Both cyclic voltammetry (CV) and differential pulse voltammetry (DPV) were performed at room temperature with a scan rate of 25 mV/s using different L-lactate concentrations.

Sequences of the LDH Constructs Used in this Study

LDH
(SEQ ID NO: 39)
ATGAAATACCTGCTGCCGACCGCTGCTGCTGGTCTGCTGCTCCTCGCTGCCCAGCC
GGCGATGGCCATGGGTGAAACTAAGGAAGATATCGCTAGAAAAGAACAACTAAAATCGC
TGCTACCTCCTCTAGATAATATTATTAACCTTTACGACTTTGAATACTTGGCCTCTCAAACT
TTGACTAAACAAGCGTGGGCCTACTATTCCTCCGGTGCTAACGACGAAGTTACTCACAGA
GAAAACCATAATGCTTATCATAGGATTTTTTTCAAACCAAAGATCCTTGTAGATGTACGCA
AAGTAGACATTTCAACTGACATGTTGGGTTCTCATGTGGATGTTCCCTTCTACGTGTCTGC
TACAGCTTTGTGTAAACTGGGAAACCCCTTAGAAGGTGAAAAAGATGTCGCCAGAGGTTG
TGGCCAAGGTGTGACAAAAGTCCCACAAATGATATCTACTTTGGCTTCATGTTCCCCTGAG
GAAATTATTGAAGCAGCACCCTCTGATAAACAAATTCAATGGTACCAACTATATGTTAAC
TCTGATAGAAAGATCACTGATGATTTGGTTAAAAATGTAGAAAAGCTGGGTGTAAAGGCA
TTATTTGTCACTGTGGATGCTCCAAGTTTAGGTCAAAGAGAAAAAGATATGAAGCTGAAA
TTTTCCAATACAAAGGCTGGTCCAAAAGCGATGAAGAAAACTAATGTAGAAGAATCTCAA
GGTGCTTCGAGAGCGTTATCAAAGTTTATTGACCCCTCTTTGACTTGGAAAGATATAGAA
GAGTTGAAGAAAAAGACAAAACTACCTATTGTTATCAAAGGTGTTCAACGTACCGAAGAT
GTTATCAAAGCAGCAGAAATCGGTGTAAGTGGGGTGGTTCTATCCAATCATGGTGGTAGA
CAATTAGATTTTTCAAGGGCTCCCATTGAAGTCCTGGCTGAAACCATGCCAATCCTGGAA
CAACGTAACTTGAAGGATAAGTTGGAAGTTTTCGTGGACGGTGGTGTTCGTCGTGGTACA
GATGTCTTGAAAGCGTTATGTCTAGGTGCTAAAGGTGTTGGTTTGGGTAGACCATTCTTGT
ATGCGAACTCATGCTATGGTCGTAATGGTGTTGAAAAAGCCATTGAAATTTTAAGAGATG
AAATTGAAATGTCTATGAGACTATTAGGTGTTACTAGCATTGCGGAATTGAAGCCTGATC
TTTTAGATCTATCAACACTAAAGGCAAGAACAGTTGGAGTACCAAACGACGTGCTGTATA
ATGAAGTTTATGAGGGACCTACTTTAACAGAATTTGAGGATGCAGGCAGTGGTTCCGGCC
ATCACCATCACCATCACTAA.
LDH-CytB
(SEQ ID NO: 40)
ATGAAATACCTGCTGCCGACCGCTGCTGCTGGTCTGCTGCTCCTCGCTGCCCAGCC
GGCGATGGCCATGGAGCCGAAACTGGATATGAATAAACAAAAGATTTCGCCCGCTGAAG
TTGCCAAGCATAACAAGCCCGATGATTGTTGGGTTGTGATCAATGGTTACGTATACGACTT
AACGCGATTCCTACCAAATCATCCAGGTGGGCAGGATGTTATCAAGTTTAACGCCGGGAA
AGATGTCACTGCTATTTTTGAACCACTACATGCTCCTAATGTCATCGATAAGTATATAGCT
CCCGAGAAAAAATTGGGTCCCCTTCAAGGATCCATGCCTCCTGAACTTGTCTGTCCTCCTT
ATGCTCCTGGTGAAACTAAGGAAGATATCGCTAGAAAAGAACAACTAAAATCGCTGCTAC
CTCCTCTAGATAATATTATTAACCTTTACGACTTTGAATACTTGGCCTCTCAAACTTTGACT
AAACAAGCGTGGGCCTACTATTCCTCCGGTGCTAACGACGAAGTTACTCACAGAGAAAAC
CATAATGCTTATCATAGGATTTTTTTCAAACCAAAGATCCTTGTAGATGTACGCAAAGTAG
ACATTTCAACTGACATGTTGGGTTCTCATGTGGATGTTCCCTTCTACGTGTCTGCTACAGC
TTTGTGTAAACTGGGAAACCCCTTAGAAGGTGAAAAAGATGTCGCCAGAGGTTGTGGCCA
AGGTGTGACAAAAGTCCCACAAATGATATCTACTTTGGCTTCATGTTCCCCTGAGGAAATT
ATTGAAGCAGCACCCTCTGATAAACAAATTCAATGGTACCAACTATATGTTAACTCTGAT
AGAAAGATCACTGATGATTTGGTTAAAAATGTAGAAAAGCTGGGTGTAAAGGCATTATTT
GTCACTGTGGATGCTCCAAGTTTAGGTCAAAGAGAAAAAGATATGAAGCTGAAATTTTCC
AATACAAAGGCTGGTCCAAAAGCGATGAAGAAAACTAATGTAGAAGAATCTCAAGGTGC
TTCGAGAGCGTTATCAAAGTTTATTGACCCCTCTTTGACTTGGAAAGATATAGAAGAGTTG
AAGAAAAAGACAAAACTACCTATTGTTATCAAAGGTGTTCAACGTACCGAAGATGTTATC
AAAGCAGCAGAAATCGGTGTAAGTGGGGGGTTCTATCCAATCATGGTGGTAGACAATTA
GATTTTTCAAGGGCTCCCATTGAAGTCCTGGCTGAAACCATGCCAATCCTGGAACAACGT
AACTTGAAGGATAAGTTGGAAGTTTTCGTGGACGGTGGTGTTCGTCGTGGTACAGATGTC
TTGAAAGCGTTATGTCTAGGTGCTAAAGGTGTTGGTTTGGGTAGACCATTCTTGTATGCGA
ACTCATGCTATGGTCGTAATGGTGTTGAAAAAGCCATTGAAATTTTAAGAGATGAAATTG
AAATGTCTATGAGACTATTAGGTGTTACTAGCATTGCGGAATTGAAGCCTGATCTTTTAGA
TCTATCAACACTAAAGGCAAGAACAGTTGGAGTACCAAACGACGTGCTGTATAATGAAGT
TTATGAGGGACCTACTTTAACAGAATTTGAGGATGCAGGCAGTGGTTCCGGCCATCACCA
TCACCATCACTAA.
LDH-CytC
(SEQ ID NO: 41)
ATGAAATACCTGCTGCCGACCGCTGCTGCTGGTCTGCTGCTCCTCGCTGCCCAGCC
GGCGATGGCCATGATTCGTGCAGGTGCTACCATGCCGCATCGTGATCGTGGTCCGTGCGG
TGCATGTCACGCTATTATCCAGGAATTCGGTTCTGGTTATGGCTCTGGTCCGCCGGGTCCG
GGTGAAACTAAGGAAGATATCGCTAGAAAAGAACAACTAAAATCGCTGCTACCTCCTCTA
GATAATATTATTAACCTTTACGACTTTGAATACTTGGCCTCTCAAACTTTGACTAAACAAG
CGTGGGCCTACTATTCCTCCGGTGCTAACGACGAAGTTACTCACAGAGAAAACCATAATG
CTTATCATAGGATTTTTTTCAAACCAAAGATCCTTGTAGATGTACGCAAAGTAGACATTTC
AACTGACATGTTGGGTTCTCATGTGGATGTTCCCTTCTACGTGTCTGCTACAGCTTTGTGT
AAACTGGGAAACCCCTTAGAAGGTGAAAAAGATGTCGCCAGAGGTTGTGGCCAAGGTGT
GACAAAAGTCCCACAAATGATATCTACTTTGGCTTCATGTTCCCCTGAGGAAATTATTGAA
GCAGCACCCTCTGATAAACAAATTCAATGGTACCAACTATATGTTAACTCTGATAGAAAG
ATCACTGATGATTTGGTTAAAAATGTAGAAAAGCTGGGTGTAAAGGCATTATTTGTCACT
GTGGATGCTCCAAGTTTAGGTCAAAGAGAAAAAGATATGAAGCTGAAATTTTCCAATACA
AAGGCTGGTCCAAAAGCGATGAAGAAAACTAATGTAGAAGAATCTCAAGGTGCTTCGAG
AGCGTTATCAAAGTTTATTGACCCCTCTTTGACTTGGAAAGATATAGAAGAGTTGAAGAA
AAAGACAAAACTACCTATTGTTATCAAAGGTGTTCAACGTACCGAAGATGTTATCAAAGC
AGCAGAAATCGGTGTAAGTGGGGTGGTTCTATCCAATCATGGTGGTAGACAATTAGATTT
TTCAAGGGCTCCCATTGAAGTCCTGGCTGAAACCATGCCAATCCTGGAACAACGTAACTT
GAAGGATAAGTTGGAAGTTTTCGTGGACGGTGGTGTTCGTCGTGGTACAGATGTCTTGAA
AGCGTTATGTCTAGGTGCTAAAGGTGTTGGTTTGGGTAGACCATTCTTGTATGCGAACTCA
TGCTATGGTCGTAATGGTGTTGAAAAAGCCATTGAAATTTTAAGAGATGAAATTGAAATG
TCTATGAGACTATTAGGTGTTACTAGCATTGCGGAATTGAAGCCTGATCTTTTAGATCTAT
CAACACTAAAGGCAAGAACAGTTGGAGTACCAAACGACGTGCTGTATAATGAAGTTTATG
AGGGACCTACTTTAACAGAATTTGAGGATGCAGGCAGTGGTTCCGGCCATCACCATCACC
ATCACTAA.
LDH-CytB-CytC
(SEQ ID NO: 42)
ATGAAATACCTGCTGCCGACCGCTGCTGCTGGTCTGCTGCTCCTCGCTGCCCAGCC
GGCGATGGCCATGATTCGTGCAGGTGCTACCATGCCGCATCGTGATCGTGGTCCGTGCGG
TGCATGTCACGCTATTATCCAGGAATTCGGTTCTGGTTATGGCTCTGGTCCGCCGGGTCCG
GAGCCGAAACTGGATATGAATAAACAAAAGATTTCGCCCGCTGAAGTTGCCAAGCATAA
CAAGCCCGATGATTGTTGGGTTGTGATCAATGGTTACGTATACGACTTAACGCGATTCCTA
CCAAATCATCCAGGTGGGCAGGATGTTATCAAGTTTAACGCCGGGAAAGATGTCACTGCT
ATTTTTGAACCACTACATGCTCCTAATGTCATCGATAAGTATATAGCTCCCGAGAAAAAAT
TGGGTCCCCTTCAAGGATCCATGCCTCCTGAACTTGTCTGTCCTCCTTATGCTCCTGGTGA
AACTAAGGAAGATATCGCTAGAAAAGAACAACTAAAATCGCTGCTACCTCCTCTAGATAA
TATTATTAACCTTTACGACTTTGAATACTTGGCCTCTCAAACTTTGACTAAACAAGCGTGG
GCCTACTATTCCTCCGGTGCTAACGACGAAGTTACTCACAGAGAAAACCATAATGCTTAT
CATAGGATTTTTTTCAAACCAAAGATCCTTGTAGATGTACGCAAAGTAGACATTTCAACTG
ACATGTTGGGTTCTCATGTGGATGTTCCCTTCTACGTGTCTGCTACAGCTTTGTGTAAACT
GGGAAACCCCTTAGAAGGTGAAAAAGATGTCGCCAGAGGTTGTGGCCAAGGTGTGACAA
AAGTCCCACAAATGATATCTACTTTGGCTTCATGTTCCCCTGAGGAAATTATTGAAGCAGC
ACCCTCTGATAAACAAATTCAATGGTACCAACTATATGTTAACTCTGATAGAAAGATCAC
TGATGATTTGGTTAAAAATGTAGAAAAGCTGGGTGTAAAGGCATTATTTGTCACTGTGGA
TGCTCCAAGTTTAGGTCAAAGAGAAAAAGATATGAAGCTGAAATTTTCCAATACAAAGGC
TGGTCCAAAAGCGATGAAGAAAACTAATGTAGAAGAATCTCAAGGTGCTTCGAGAGCGT
TATCAAAGTTTATTGACCCCTCTTTGACTTGGAAAGATATAGAAGAGTTGAAGAAAAAGA
CAAAACTACCTATTGTTATCAAAGGTGTTCAACGTACCGAAGATGTTATCAAAGCAGCAG
AAATCGGTGTAAGTGGGGTGGTTCTATCCAATCATGGTGGTAGACAATTAGATTTTTCAA
GGGCTCCCATTGAAGTCCTGGCTGAAACCATGCCAATCCTGGAACAACGTAACTTGAAGG
ATAAGTTGGAAGTTTTCGTGGACGGTGGTGTTCGTCGTGGTACAGATGTCTTGAAAGCGTT
ATGTCTAGGTGCTAAAGGTGTTGGTTTGGGTAGACCATTCTTGTATGCGAACTCATGCTAT
GGTCGTAATGGTGTTGAAAAAGCCATTGAAATTTTAAGAGATGAAATTGAAATGTCTATG
AGACTATTAGGTGTTACTAGCATTGCGGAATTGAAGCCTGATCTTTTAGATCTATCAACAC
TAAAGGCAAGAACAGTTGGAGTACCAAACGACGTGCTGTATAATGAAGTTTATGAGGGA
CCTACTTTAACAGAATTTGAGGATGCAGGCAGTGGTTCCGGCCATCACCATCACCATCAC
TAA.

### Results and Discussion

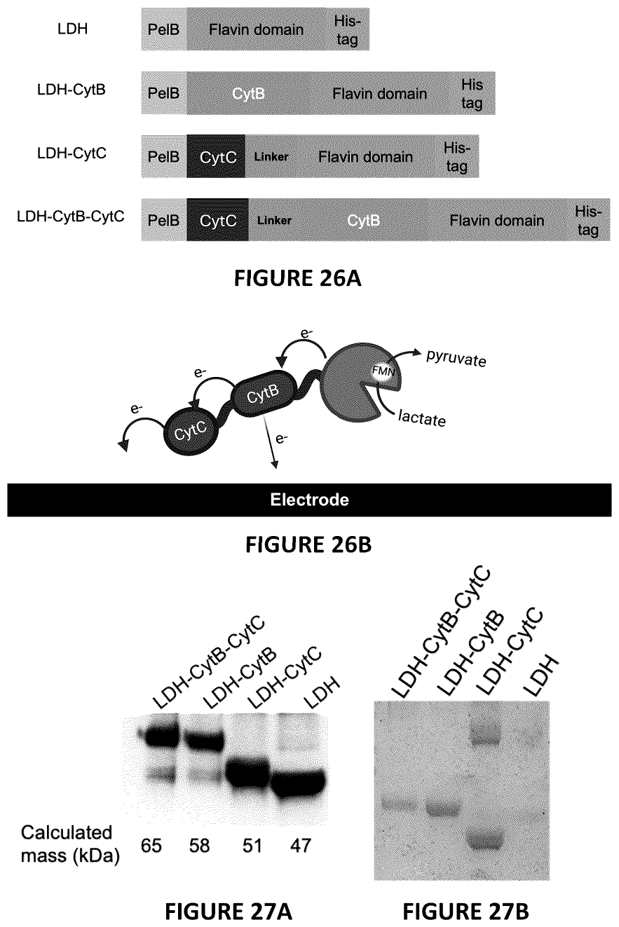

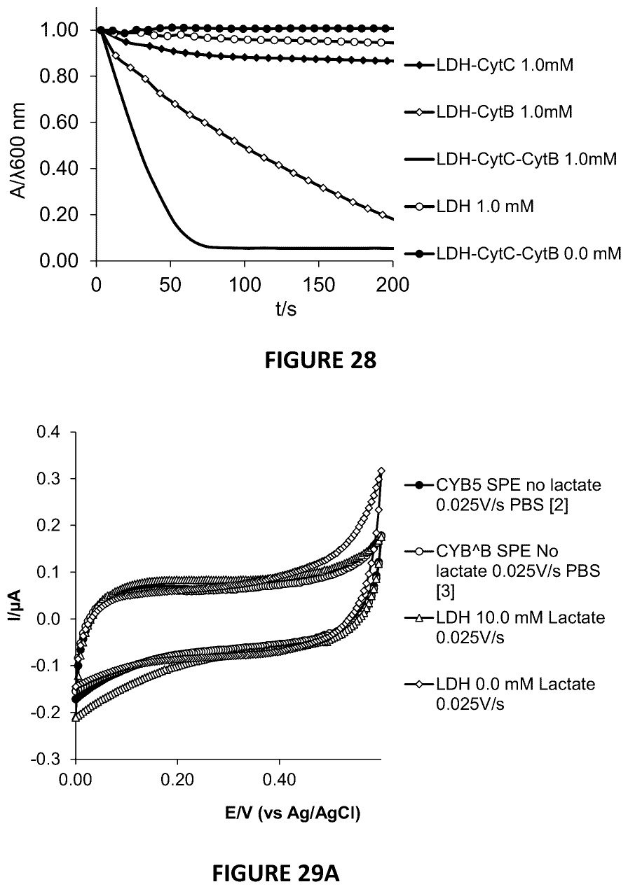

Four different constructs of LDH were prepared (FIG. 26A), namely, native LDH lacking its CytB (termed LDH), native LDH with its CytB (termed LDH-CytB), LDH with MCD fused to the CytB N-terminus (termed “LDH-CytB-CytC”) and LDH containing MCD only (termed LDH-CytC). The constructs, expressed along with a cytochrome maturation gene cassette, included a PelB signal peptide sequence to direct the expressed proteins to the E. coli periplasm. Successful expression of the different constructs was verified by western blot and heme staining, to verify maturation of the heme group in each case (FIGS. 27A and 27B, respectively). The heme staining gel and the absorbance spectra of the variants (FIGS. 28 and 33) allowed the inventors to confirm that only cytochrome-containing proteins were stained and had matured with a porphyrin. It was also seen that in the case of the cytochrome c fusion construct (i.e., LDH-CytC), a stable dimer had formed that did not disassemble under denaturing SDS-PAGE conditions.

After verification that all four LDH variants were expressed, their catalytic activities and their Michaelis constants (K_{M}) toward lactate were next addressed. This was achieved in an assay relying on di-chloroindophenol (DCIP) as final electron acceptor, the blue color (λ=600 nm) of which disappears upon its reduction (for details see Materials section above). Michaelis-Menten and Linweaver-Burk plots were then used to calculate kinetic constants (FIG. 34). Table 5 summarizes the results of these measurements. Representative absorbance spectra of DCIP reduction in the presence of 1 mM lactate for the different variants are presented in FIG. 28. Table 5 and FIG. 28 show that the fastest electron-transferring enzyme is that variant containing two cytochromes, i.e., LDH-CytB-CytC. The second fastest electron-transferring variant was the native enzyme that contains cytochrome b2 only, while the third fastest enzyme was that variant contains cytochrome c only. Nonetheless, this variant (i.e., LDH-CytB) had a k_{cat} some five-fold higher than the variant that does not contain cytochrome b or c (i.e., LDH). The variant containing cytochrome c only (i.e., LDH-CytC) was five-fold slower that the native enzyme, likely due to the lower reduction potential of the variant, which is less compatible with the reduction potential of the flavin mononucleotide (FMN). The FMN in the native enzyme is near the heme of cytochrome b2, which present very close redox potentials to each another (i.e., approximately +5-(−58) mV and +8 mV versus a standard hydrogen electrode (SHE), respectively). The introduced cytochrome c has a slightly lower redox potential of around −76 mV versus a SHE.

TABLE 5
Michaelis-Menten and apparent kinetic constants of the different constructs
using DCIP as the final electron acceptor

| Variant | K_{M}^{app} (mM) | K_{cat}^{app} [s⁻¹] | K_{cat}/K_{M} [mM*s] |
|---|---|---|---|
| LDH | 0.10 ± 0.02 | 0.04 ± 0.005 | 0.4 |
| LDH-CytB (native enzyme) | 0.52 ± 0.03 | 1.04 ± 0.07 | 2.0 |
| LDH-CytC | 0.17 ± 0.07 | 0.20 ± 0.02 | 1.2 |
| LDH-CytB-CytC | 0.55 ± 0.04 | 13.20 ± 0.02 | 24 |

From the Michaelis constants (K_{M}), it was concluded that the fusion of MCD did not interfere with substrate recognition as the K_{M} values of the native enzyme and the LDH-CytB-CytC variant were very close. Furthermore, given the lower K_{M} values measured with LDH lacking any cytochrome (i.e., LDH) and that variant containing cytochrome c only (i.e., LDH-CytC), it would seem that substrate affinity even improved by some five-fold for both, relative to the native enzyme (i.e. LDH-CytB), probably due to a removal of interference between cytochrome b2 and the substrate and product entry and exit channels in the enzyme. When comparing the kinetic properties resulting from ET to the final electron acceptor DCIP, there was a significant increase in the enzyme kinetics of the dual-cytochrome variant with k_{cat} being 13-fold higher and the catalytic efficiency being 12-fold higher than those of the native enzyme (LDH-CytB). This is indicative of the fusion of MCD not hindering enzyme activity or tetramer formation, instead significantly improving the activity of the enzyme towards lactate and the final electron transfer-step to cytochrome c. In the native yeast system, a separate cytochrome c in mitochondria serves as final electron acceptor (along with a cytochrome oxidase) and is a rate-limiting step in the ET pathway involving LDH.

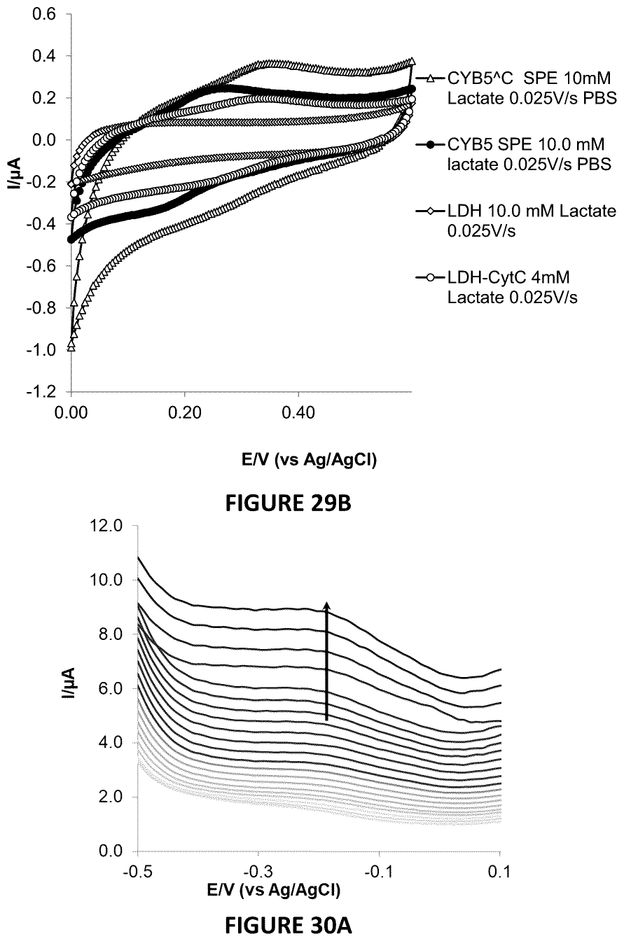

The constructs were next considered in terms of ET to an electrode. This was addressed using a carbon-nanotube-coated screen-printed electrode (SPE) with a pseudo-Ag/AgCl reference electrode, using similar concentrations of the four enzyme variants that drop-casted onto the electrodes. CV and DPV were first used to identify the relevant peaks originating from the cytochromes in the constructs (if any) and to determine the reduction and oxidation peaks, as well as the relative potential shifts of each construct. FIG. 29A shows the CVs of all constructs in the absence of lactate after drop-casting them onto the electrodes. The scan rates are 25 mV/s. FIG. 29B shows voltammograms in the presence of 5 mM lactate. It can be clearly seen that all variants, other than LDH, showed peaks stemming from the presence of the cytochromes. However, these peaks were visible in the CVs only in the presence of the substrate. The peaks for all variants appeared at very high potentials, relative to the values reported in the literature for both cytochrome b2 and cytochrome c. The oxidation and reduction peaks measured for the three constructs that contained a cytochrome at two different lactate concentrations are summarized in Table 6.

TABLE 6
Apparent reduction/oxidation potential peaks of LDH constructs vs.
Ag/AgCl reference electrode.

| | Lactate | | | |
|---|---|---|---|---|
| | 5 mM | | 1 mM | |
| | pH | | | |
| | 6.50 | | 7.21 | |
| | Variant | | | |
| | E^{0app}_{oxidation} | ^{E0app}_{reduction} | E^{0app}_{oxidation} | ^{E0app}_{reduction} |
| LDH | — | — | — | — |
| LDH-CytB | 183 | 121 | 120 | 28 |
| LDH-CytC | 339 | 238 | 220 | 38 |
| LDH-CytB-CytC | 232 | 150 | 140 | 24 |

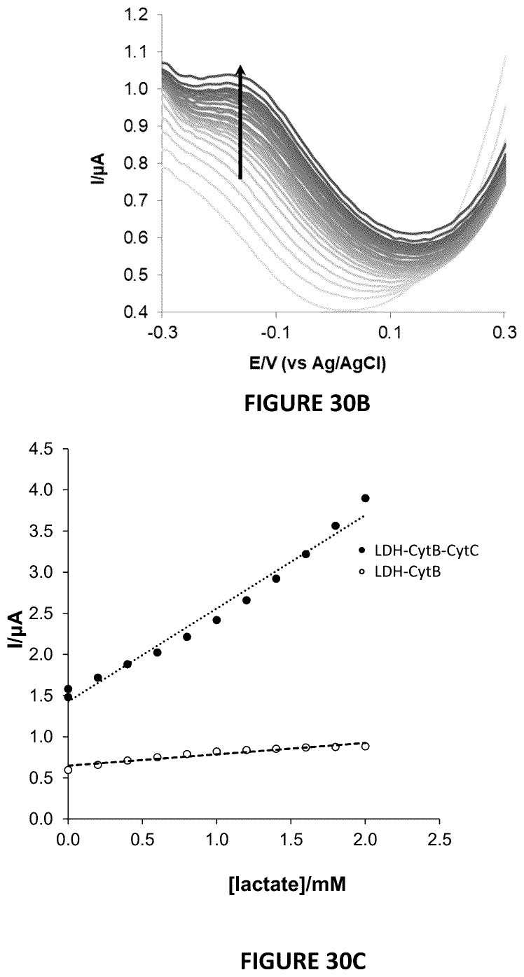

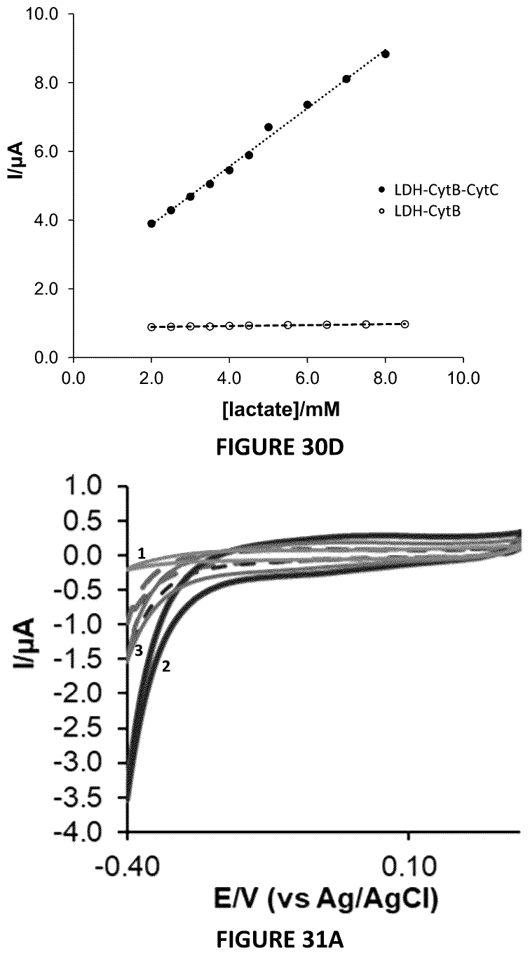

It was speculated that the peaks were visible at such high potentials because the phosphate buffer that was used was not strong enough to buffer high concentrations of externally added lactic acid, and as such, the peaks positively shifted with the decrease in pH. To test this notion, a pH-dependence study in the absence of the substrates, but with drop-casted enzyme on the electrodes, was conducted. This revealed that the drastic shifts in the pH of the solution indeed resulted in a shift in the potentials of the reduction and oxidation peaks of 79 mV per pH unit. FIG. 29B shows a linear pH dependence of the oxidation peaks. Accordingly, subsequent DPV experiments on the constructs were performed in a 100 mM phosphate buffer, rather than the 50 mM phosphate buffer used in the less sensitive CV assays (FIGS. 30A-30B). Since those enzyme constructs with cytochrome c alone or without any cytochrome showed lower activity than did the other two constructs, we characterized only the wild-type construct (i.e., LDH-CytB) and the dual-cytochrome construct (i.e., LDH-CytB-CytC). From the voltammogram of LDH that does not contain cytochrome, it was deduced that there was no direct electron transfer (DET) for this enzyme variant in the absence of cytochromes. DPV efforts also revealed no reduction peak for the same construct at lower potential ranges, indicative that the LDH variant does not perform DET, despite its catalytic site remaining intact on the electrode.

Upon establishing that the enzyme variants did not lose activity on the electrode, nor did they lose activity due to exposure to high substrate concentrations, with low pH values resulting, the reliability of the variants in lactate biosensing was next assessed. Specifically, efforts focused on the two most active enzyme namely, the novel fusion enzyme LDH-CytB-CytC and the native enzyme, LDH-CytB. FIG. 29 shows DPVs upon addition of varying lactate concentrations to the electrode, with FIG. 29A showing the DPVs of LDH-CytB-CytC and FIG. 29B showing the DPVs of LDH-CytB. The increase in bioelectrocatalytic current was larger for LDH-CytB-CytC than that of LDH-CytB, and appeared at a lower potential (E⁰′) than that of the native enzyme (−180 mV and −157 mV, respectively), which are reasonable values for the potential difference between the two variants and the reported values of redox potentials of the two cytochromes. In addition, one would expect an average potential for the dual-cytochrome construct, given how the redox potential of each cytochrome contributes to the redox potential of the entire construct and since the enzyme is randomly oriented on the electrode. FIGS. 30C-30D present calibration curves that were generated for the maximum oxidation currents of the two constructs. As expected, a saturation curve was obtained for the native construct since at higher lactate concentrations, pyruvate accumulation binds to the semi-quinone of the reduced FMN in some of the sub-units of the tetramer, both driving a shift in the potential while also inhibiting the enzyme. This, for a reason unknown to us, does not occur in the high lactate concentration range in the dual-cytochrome construct, where saturation was not reached even in the presence of 8-10 mM of lactate. At both lactate ranges tested, it could be clearly seen that the enzyme variant with two cytochromes was much more active than was that variant with cytochrome b2 alone.

When performing CV measurements, an evolution of reductive currents that was dependent on lactate concentrations was noted. As such, it was hypothesized that upon oxidation of lactate and reduction of FMN and subsequently of both cytochromes in the presence of oxygen, oxygen reduction by the enzyme to water or most probably according to the reduction potential the inventors observed to hydrogen peroxide (for a list of reduction potentials, see table 7 below) (depending on the local pH) had also occurred in a lactate concentration-dependent manner.

TABLE 7
*Reduction potentials of relevant species

| Reaction | E (vs. Ag/AgCl)/mV |
|---|---|
| FMN + 2e⁻↔FMNH₂³ | (−192) − (−255) |
| 2 cytochrome b₍ₒₓ₎ + 2e⁻↔2 cytochrome b_{(red)}³ | −189 |
| 2 cytochrome c₍ₒₓ₎ + 2e⁻↔2 cytochrome c_{(red)}¹, 41, | −286 |
| pH = 7.5 | |
| Lactate + 2e⁻↔Pyruvate⁴ | −197 |
| O₂ + 2e⁻ + 2H⁺ ↔ H₂O₂5 | −500 |
| O₂ + 4e⁻ + 4H⁺ ↔ 2H₂O5 | −1000 |

*All potentials reported are at 25° C., pH = 7, unless stated otherwise. E vs. Ag/AgCl was calculated by the following equation: E_{Ag/AgCl} = E_{RHE} - 197 mV

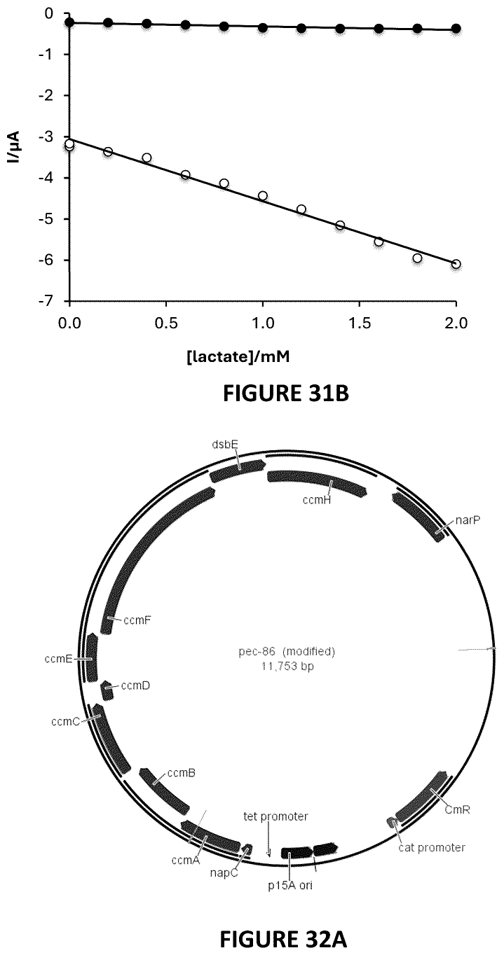

To test this concept, calibration curves from the electro/biocatalytic reduction peaks generated by both enzymes were created. FIG. 31 shows the result of such efforts, with FIG. 31A reflecting CVs generated when attempting to reduce oxygen in the presence or absence of 8 mM lactate. In the absence of lactate, both enzyme constructs (i.e., LDH-CytB and LDH-CytB-CytC) reduced oxygen although LDH-CytB did so to a lesser extent, as opposed to the dual-cytochrome construct, which was more efficient. This difference was more prominent when 8 mM of lactate were added. While the changes in current with the native enzyme were minute, the evolved bioelectrocatalytic reduction current for the LDH-CytB-CytC construct was more obvious. FIG. 31B shows calibration curves constructed for the different lactate concentrations added to both enzymes. In the oxygen reduction experiment, although there was still a marked difference in the currents for the dual-cytochrome construct, linearity was maintained in the 0-2 mM lactate range even after adding 10 mM of lactate. The same was true for the native enzyme, although a very small slope in the calibration curve was detected, indicating very low sensitivity and sensing resolution of potential oxygen-sensing abilities. Taken together, it was concluded that the DET abilities of the dual-cytochrome enzyme were much improved, as compared to the single CytB2-containing native enzyme. To further validate this result, the experiment was repeated in an almost oxygen-deficient environment (i.e., under argon atmosphere). The findings here confirmed the earlier observation, with no visible electrocatalytic reduction peaks being observed in the presence or absence of lactate under the anaerobic atmosphere (FIG. 31A, 1).

### CONCLUSIONS

Addition of a cytochrome c with a prosthetic heme group covalently attached to the cytochrome domain of S. cerevisiae LDH proved to be highly efficient in improving the ET pathway of electrons released upon lactate oxidation. An almost 13-fold increase in the apparent catalytic rate constant was determined for this LDH variant over that of the native enzyme (i.e. LDH-CytB). When these two versions of the enzyme were deposited onto an electrode without any specific orientation, the di-cytochrome fusion enzyme demonstrated superior activity, showing 12-fold higher activity than did the native enzyme. This was especially true at the higher lactate concentrations considered, thus rendering LDH-CytB-CytC a suitable candidate for monitoring high levels of physiological lactate concentrations, e.g., in sweat.

In addition, serendipitously the inventors found that the new fusion enzyme is very efficient in the lactate dependent reduction of oxygen in low overpotentials, and could probably be extended to the reduction of other molecules and metal ions in a similar way. This is done through a mechanism that is yet unknown, but due to the control experiment with the construct with cytochrome b only which did not exhibit similar dependence, the inventors speculate that the reaction depends on the presence of the cytochrome c maybe due to its lower redox potentials. This mechanism should be further investigated in a follow-up study.

All publications, patents and patent applications mentioned in this specification are herein incorporated in their entirety by reference into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

What is claimed is:

1. A recombinant protein, comprising (a) lactate dehydrogenase (LDH); and (b) a minimal c-type cytochrome peptide.

2. The recombinant protein of claim 1, characterized by a Michaelis-Menten constant (K_{M}^{app}) ranging between 0.45 and 0.65 mM.

3. The recombinant protein of claim 1, characterized by a catalytic constant (k_{cat}^{app}) ranging between 10 and 15 [Sec]⁻¹.

4. The recombinant protein of claim 1, wherein said LDH is Saccharomyces cerevisiae LDH.

5. The recombinant protein of claim 1, further comprising a linker.

6. The recombinant protein of claim 1, further being bound to a porphyrin comprising a metal.

7. A polynucleotide comprising a nucleic acid sequence encoding the recombinant protein of claim 1.

8. The polynucleotide of claim 7, comprising a nucleic acid sequence set forth in SEQ ID NO: 42.

9. The polynucleotide of claim 8, being operably linked to a regulatory element.

10. The polynucleotide of claim 9, wherein said regulatory element is a T7 promoter.

11. An expression vector or a plasmid comprising the polynucleotide of claim 7.

12. A transgenic or a transfected cell comprising the polynucleotide of claim 7.

13. The transgenic or transfected cell of claim 12, being a prokaryotic cell.

14. An extract obtained or derived from the transgenic or transfected cell of claim 13.

15. A composition comprising the recombinant protein of claim 1, and an acceptable carrier.

16. An electrode coupled to the recombinant protein of claim 1, wherein said coupled is by non-covalent interactions.

17. A device comprising the electrode of claim 16.

18. A method for determining presence, concentration, or both, of an analyte in a liquid medium, the analyte being capable of undergoing a biocatalytic oxidation or reduction reaction in the presence of an oxidizer or a reducer, respectively, the method comprising:
i. providing said device of claim 17;
ii. contacting said device with said liquid medium;
iii. measuring the electric signal generated between a cathode and an anode, the electric signal being indicative of any one of the presence of the analyte, the concentration of the analyte, and both; and
iv. determining the presence, concentration, or both, of said analyte in said liquid medium based on the electric signal measured in step (iii).

19. The method of claim 18, wherein said analyte comprises lactate.
